SEQUENCE LISTING (183 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 4 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 5 - protein, 21 aa, Homo sapiens
GIVDECCFRSCDLRRLEMYCA

SEQ ID NO: 6 - protein, 30 aa, Homo sapiens
FGPETLCGAELVDALYLVCGDRGFYFNKPT

SEQ ID NO: 7 - protein, 21 aa, Homo sapiens
GIVEECCFRSCDLALLETYCA

SEQ ID NO: 8 - protein, 32 aa, Homo sapiens
AYRPSETLCGGELVDTLQFVCGDRGFYFSRPA

SEQ ID NO: 9 - protein, 8 aa, Artificial Sequence
  spacer sequence
  MISC_FEATURE (1)..(1): The Xaa at position 1 represents an amino acid selected from glycine, alanine, valine, leucine, isoleucine, proline, phenylalanine and methionine
  MISC_FEATURE (2)..(2): The Xaa at position 2 represents any amino acid other than tyrosine
  MISC_FEATURE (3)..(6): The Xaas at positions 3-6 are independently any amino acid
  MISC_FEATURE (7)..(8): The Xaas at positions 7 and8 are independently selected from arginine, lysine or ornithine
XXXXXXXX

SEQ ID NO: 10 - protein, 8 aa, Artificial Sequence
  spacer sequence
  MISC_FEATURE (1)..(1): The Xaa at position 1 represents an amino acid selected from glycine, alanine, valine, leucine, isoleucine, proline, phenylalanine and methionine
  MISC_FEATURE (2)..(2): The Xaa at position 2 represents any amino acid other than tyrosine
  MISC_FEATURE (3)..(6): The Xaas at positions 3-6 are independently any amino acid
XXXXXXRR

SEQ ID NO: 11 - protein, 4 aa, Artificial Sequence
  spacer sequence
PGPE

SEQ ID NO: 12 - protein, 4 aa, Artificial Sequence
  spacer sequence
FVNQ

SEQ ID NO: 13 - protein, 5 aa, Artificial Sequence
  spacer sequence
  MISC_FEATURE (1)..(5): The Xaas at positions 1-5 are independently any amino acid
XXXXX

SEQ ID NO: 14 - protein, 6 aa, Artificial Sequence
  spacer sequence
AYRPSE

SEQ ID NO: 15 - protein, 4 aa, Artificial Sequence
  spacer sequence
  MISC_FEATURE (1)..(4): The Xaas at positions 1-4 are independently any amino acid
XXXX

SEQ ID NO: 16 - protein, 5 aa, Artificial Sequence
  spacer sequence
YTPKT

SEQ ID NO: 17 - protein, 12 aa, Homo sapiens
GYGSSSRRAPQT

SEQ ID NO: 18 - protein, 8 aa, Homo sapiens
GYGSSSRR

SEQ ID NO: 19 - protein, 21 aa, Artificial Sequence
  Analog of the insulin A chain
  MISC_FEATURE (4)..(4): Xaa at position 5 is aspartic acid or glutamic acid
  MISC_FEATURE (5)..(5): Xaa at position 5 is glutamine or glutamic acid
  MISC_FEATURE (8)..(8): Xaa at position 8 is histidine, threonine or phenylalanine
  MISC_FEATURE (9)..(9): Xaa at position 9 is serine, arginine, ornithine, lysine or alanine
  MISC_FEATURE (10)..(10): Xaa at position 10 is isoleucine or serine
  MISC_FEATURE (12)..(12): Xaa at position 12 is aspartic acid or serine
  MISC_FEATURE (14)..(14): Xaa at position 14 is tyrosine, arginine, ornathine, lysine or alanine
  MISC_FEATURE (15)..(15): Xaa at position 15 is glutamine, glutamic acid, arginine, ornithine, alanine, lysine or leucine
  MISC_FEATURE (17)..(17): Xaa at position 17 is glutamine or glutamic acid
  MISC_FEATURE (18)..(18): Xaa at position 18 is methionine, asparagine, glutamine, glutamic acid, aspartic acid or threonine
  MISC_FEATURE (21)..(21): Xaa at position 21 is alanine, glycine, serine, valine, threonine, isoleucine, leucine, glutamine, glutamic acid, asparagine, aspartic acid, histidine, tryptophan, tyrosine, or methionine
GIVXXCCXXXCXLXXLXXYCX

SEQ ID NO: 20 - protein, 21 aa, Artificial Sequence
  Analog of the insulin B chain
  MISC_FEATURE (1)..(1): Xaa at position 1 is histidine or threonine
  MISC_FEATURE (5)..(5): Xaa at position 5 is alanine, glycine or serine
  MISC_FEATURE (6)..(6): Xaa at position 6 is histidine, aspartic acid, glutamic acid, homocysteic acid or cysteic acid
  MISC_FEATURE (9)..(9): Xaa at position 9 is aspartic acid, glutamine or glutamic acid
  MISC_FEATURE (10)..(10): Xaa at position 10 is alanine or threonine
  MISC_FEATURE (17)..(17): Xaa at position 12 is glutamic acid, aspartic acid or asparagine
  MISC_FEATURE (18)..(18): Xaa at position 18 is alanine, ornithine, lysine or arginine
  MISC_FEATURE (21)..(21): Xaa at position 21 is tyrosine or 4-amino-phenylalanine
XLCGXXLVXXLYLVCGXXGFX

SEQ ID NO: 21 - protein, 8 aa, Artificial Sequence
  analog of IGF-1 C peptide
  MISC_FEATURE (7)..(8): The Xaas at positions 7 and 8 are independently arginine, lysine or ornithine
GYGSSSXX

SEQ ID NO: 22 - protein, 8 aa, Artificial Sequence
  Analog of IGF-1 C peptide
GAGSSSRR

SEQ ID NO: 23 - protein, 12 aa, Artificial Sequence
  linker sequence
GAGSSSRRAPQT

SEQ ID NO: 24 - protein, 8 aa, Homo sapiens
SRVSRRSR

SEQ ID NO: 25 - protein, 21 aa, Artificial Sequence
  Analog of the insulin B chain
  MISC_FEATURE (1)..(1): Xaa at position 1 is histidine or threonine
  MISC_FEATURE (5)..(5): Xaa at position 5 is alanine, glycine or serine
  MISC_FEATURE (6)..(6): Xaa at position 6 is histidine, aspartic acid, glutamic acid, homocysteic acid or cysteic acid
  MISC_FEATURE (9)..(9): Xaa at position 9 is aspartic acid or glutamic acid
  MISC_FEATURE (10)..(10): Xaa at position 10 is alanine or threonine
  MISC_FEATURE (18)..(18): Xaa at position 18 is alanine, ornithine, lysine or arginine
  MISC_FEATURE (21)..(21): Xaa at position 21 is tyrosine or phenylalanine
XLCGXXLVXXLYLVCGDXGFX

SEQ ID NO: 26 - protein, 7 aa, Artificial Sequence
  amino acid terminal extension for insulin
  MISC_FEATURE (2)..(6): the Xaas at positions 2-6 are independently glutamic acid or aspartic acid
GXXXXXK

SEQ ID NO: 27 - protein, 7 aa, Artificial Sequence
  amio acid terminal extension for insulin
  MISC_FEATURE (1)..(5): the Xaas at positions 1-5 are independently glutamic acid or aspartic acid
XXXXXRK

SEQ ID NO: 28 - protein, 31 aa, Artificial Sequence
  Analog of IGF-1 B chain
  MISC_FEATURE (28)..(28): The Xaa at position 28 is alanine lysine, ornithine or arginine
GEEEEEKGPEHLCGAHLVDALYLVCGDXGFY

SEQ ID NO: 29 - protein, 21 aa, Artificial Sequence
  Analog of the insulin A chain
  MISC_FEATURE (4)..(4): Xaa at position 5 is aspartic acid or glutamic acid
  MISC_FEATURE (5)..(5): Xaa at position 5 is glutamine or glutamic acid
  MISC_FEATURE (8)..(8): Xaa at position 8 is histidine, threonine or phenylalanine
  MISC_FEATURE (9)..(9): Xaa at position 9 is serine, arginine, ornithine, lysine or alanine
  MISC_FEATURE (10)..(10): Xaa at position 10 is isoleucine or serine
  MISC_FEATURE (12)..(12): Xaa at position 12 is aspartic acid or serine
  MISC_FEATURE (14)..(14): Xaa at position 14 is tyrosine, arginine, ornathine, lysine or alanine
  MISC_FEATURE (15)..(15): Xaa at position 15 is glutamine, glutamic acid, arginine, ornithine, alanine, lysine or leucine
  MISC_FEATURE (17)..(17): Xaa at position 17 is glutamine or glutamic acid
  MISC_FEATURE (18)..(18): Xaa at position 18 is methionine, asparagine, glutamine, glutamic acid, aspartic acid or threonine
  MISC_FEATURE (19)..(19): Xaa at position19 is tyrosine, 4-methoxy phenylalanine or 4-amino-phenylalanine
  MISC_FEATURE (21)..(21): Xaa at position 21 is alanine, glycine, serine, valine, threonine, isoleucine, leucine, glutamine, glutamic acid, asparagine, aspartic acid, histidine, tryptophan, tyrosine, or methionine
GIVXXCCXXXCXLXXLXXXCX

SEQ ID NO: 30 - protein, 21 aa, Artificial Sequence
  Analog of insulin A chain
  MISC_FEATURE (8)..(8): Xaa at position 8 is  threonine or histidine
  MISC_FEATURE (19)..(19): Xaa at position 19 is tyrosine, 4-methoxy-phenylalanine or 4-amino phenylalanine
  MISC_FEATURE (21)..(21): Xaa at position 21 is asparagine or glycine
GIVEQCCXSICSLYQLENXCX

SEQ ID NO: 31 - protein, 21 aa, Artificial Sequence
  Analog of insulin B chain
  MISC_FEATURE (1)..(1): Xaa at position 1 is histidine or threonine
  MISC_FEATURE (5)..(5): Xaa at position 5 is alanine, glycine or serine
  MISC_FEATURE (6)..(6): Xaa at position 6 is  histidine, aspartic acid, glutamic acid, homocysteic acid or cysteic acid
XLCGXXLVEALYLVCGERGFF

SEQ ID NO: 32 - protein, 21 aa, Artificial Sequence
  Analog of the insulin A chain
  MISC_FEATURE (8)..(8): Xaa at position 8 is histidine or threonine
  MISC_FEATURE (17)..(17): Xaa at position 17 is glutamine or glutamic acid
  MISC_FEATURE (21)..(21): Xaa at position 21 is asparagine or glycine
GIVEQCCXSICSLYQLXNYCX

SEQ ID NO: 33 - protein, 21 aa, Artificial Sequence
  Analog of the insulin B chain
  MISC_FEATURE (1)..(1): Xaa at position 1 is histidine or threonine
  MISC_FEATURE (5)..(5): Xaa at position 5 is alanine, glycine or serine
  MISC_FEATURE (6)..(6): Xaa at position 6 is histidine, aspartic acid, glutamic acid, homocysteic acid or cysteic acid
XLCGXXLVEALYLVCGERGFF

SEQ ID NO: 34 - protein, 27 aa, Artificial Sequence
  Analog of the insulin B chain
  MISC_FEATURE (1)..(1): Xaa at position 1 is phenylalanine and desamino-phenylalanine
  MISC_FEATURE (5)..(5): Xaa at position 5 is histidine and threonine
  MISC_FEATURE (9)..(9): Xaa at position 9 is alanine, glycine or serine
  MISC_FEATURE (10)..(10): Xaa at position 10 is histidine, aspartic acid, glutamic acid, homocysteic acid or cysteic acid
XVNQXLCGXXLVEALYLVCGERGFFYT

SEQ ID NO: 35 - protein, 21 aa, Artificial Sequence
  Analog of the insulin A chain
  MISC_FEATURE (8)..(8): Xaa at position 8 is histidine or phenylalnine
  MISC_FEATURE (9)..(9): Xaa at position 9 is arginine, lysine, ornithine or alanine
  MISC_FEATURE (14)..(14): Xaa at position 14 is arginine, lysine, ornithine or alanine
  MISC_FEATURE (15)..(15): Xaa at position 15 is arginine, lysine, ornithine or leucine
  MISC_FEATURE (17)..(17): Xaa at position 17 is glutamine or glutamic acid
  MISC_FEATURE (18)..(18): Xaa at position 18 is methionine, asparagine or threonine
  MISC_FEATURE (19)..(19): Xaa at position19 is tyrosine, 4-methoxy phenylalanine or 4-amino-phenylalanine
  MISC_FEATURE (21)..(21): Xaa at position 21 is alanine, glycine or asparagine
GIVDECCXXSCDLXXLXXXCX

SEQ ID NO: 36 - protein, 21 aa, Artificial Sequence
  Analog of the insulin B chain
  MISC_FEATURE (1)..(1): Xaa at position 1 is histidine or threonine
  MISC_FEATURE (5)..(5): Xaa at position 5 is alanine, glycine or serine
  MISC_FEATURE (6)..(6): Xaa at position 6 is histidine, aspartic acid, glutamic acid, homocysteic acid or cysteic acid
  MISC_FEATURE (9)..(9): Xaa at position 9 is aspartic acid or glutamic acid
  MISC_FEATURE (10)..(10): Xaa at position 10 is alanine or threonine
  MISC_FEATURE (18)..(18): Xaa at position 18 is alanine, ornithine, lysine or arginine
  MISC_FEATURE (21)..(21): Xaa at position 21 is tyrosine or phenylalanine
XLCGXXLVXXLYLVCGDXGFX

SEQ ID NO: 37 - protein, 21 aa, Artificial Sequence
  Analog of the insulin A chain
  MISC_FEATURE (9)..(9): Xaa at position 9 is arginine, ornithine, or lysine
  MISC_FEATURE (14)..(14): Xaa at position 14 is arginine, ornithine, or lysine
  MISC_FEATURE (15)..(15): Xaa at position 15 is arginine, ornithine, or lysine
  MISC_FEATURE (17)..(17): Xaa at position 17 is glutamine or glutamic acid
  MISC_FEATURE (19)..(19): Xaa at position19 is tyrosine, 4-methoxy phenylalanine or 4-amino-phenylalanine
  MISC_FEATURE (21)..(21): Xaa at position 21 is alanine, glycine or asparagine
GIVDECCHXSCDLXXLXMXCX

SEQ ID NO: 38 - protein, 21 aa, Artificial Sequence
  Analog of the insulin B chain
  MISC_FEATURE (1)..(1): Xaa at position 1 is histidine or threonine
  MISC_FEATURE (6)..(6): Xaa at position 6 is histidine, aspartic acid, or glutamic acid
  MISC_FEATURE (18)..(18): Xaa at position 18 is alanine, ornithine, lysine or arginine
  MISC_FEATURE (21)..(21): Xaa at position 21 is tyrosine or phenylalanine
XLCGAXLVDALYLVCGDXGFX

SEQ ID NO: 39 - protein, 21 aa, Artificial Sequence
  analog of insulin B chain
  MISC_FEATURE (18)..(18): The Xaa at position 18 is ornithine, lysine or arginine
HLCGAELVDALYLVCGDXGFY

SEQ ID NO: 40 - protein, 24 aa, Artificial Sequence
  analog of IGF-1 B chain
  MISC_FEATURE (21)..(21): The Xaa at position 21 is ornithine, lysine or arginine
GPEHLCGAELVDALYLVCGDXGFY

SEQ ID NO: 41 - protein, 29 aa, Artificial Sequence
  Analog of IGF-1 B chain
  MISC_FEATURE (21)..(21): The Xaa at position 21 is ornithine, lysine or arginine
GPEHLCGAELVDALYLVCGDXGFYFNPKT

SEQ ID NO: 42 - protein, 29 aa, Artificial Sequence
  Analog of IGF-1 B chain
  MISC_FEATURE (21)..(21): The Xaa at position 21 is ornithine, lysine or arginine
GPEHLCGAELVDALYLVCGDXGFYFNKPT

SEQ ID NO: 43 - protein, 21 aa, Artificial Sequence
  Analog of the insulin A chain
  MISC_FEATURE (9)..(9): Xaa at position 9 is arginine, ornithine, or lysine
  MISC_FEATURE (14)..(14): Xaa at position 14 is arginine, ornithine, or lysine
  MISC_FEATURE (15)..(15): Xaa at position 15 is arginine, ornithine, or lysine
GIVDECCHXSCDLXXLQMYCN

SEQ ID NO: 44 - protein, 21 aa, Artificial Sequence
  Analog of the insulin A chain
  MISC_FEATURE (8)..(8): Xaa at position 8 is threonine, histidine or phenylalnine
  MISC_FEATURE (19)..(19): Xaa at position19 is tyrosine, 4-methoxy phenylalanine or 4-amino-phenylalanine
GIVDECCXRSCDLYQLENXCN

SEQ ID NO: 45 - protein, 21 aa, Artificial Sequence
  Analog of the insulin B chain
  MISC_FEATURE (1)..(1): Xaa at position 1 is histidine or threonine
  MISC_FEATURE (18)..(18): Xaa at position 18 is alanine, ornithine, lysine or arginine
  MISC_FEATURE (21)..(21): Xaa at position 21 is tyrosine, histidine, asparagine or phenylalanine
XLCGSHLVDALYLVCGDXGFX

SEQ ID NO: 46 - protein, 21 aa, Artificial Sequence
  Analog of the insulin A chain
  MISC_FEATURE (19)..(19): Xaa at position19 is tyrosine, 4-methoxy phenylalanine or 4-amino-phenylalanine
  MISC_FEATURE (21)..(21): Xaa at position 21 is alanine, glycine or asparagine
GIVEQCCHSICSLYQLENXCX

SEQ ID NO: 47 - protein, 21 aa, Homo sapiens
  MISC_FEATURE (19)..(19): The Xaa at position 19 is tyrosine, 4-methoxy-phenylalanine or 4-amino phenylalanine
  MISC_FEATURE (21)..(21): The Xaa at position 21 is alanine, glycine or asparagine
GIVDECCHRSCDLRRLEMXCX

SEQ ID NO: 48 - protein, 29 aa, Homo sapiens
GPETLCGAELVDALYLVCGDRGFYFNPKT

SEQ ID NO: 49 - protein, 8 aa, Artificial Sequence
  linker sequence
  MISC_FEATURE (1)..(1): Xaa at position1 is glycine, alanine, valine, leucine, isoleucine, proline, phenylalanine and methionine
  MISC_FEATURE (2)..(2): Xaa at position 2 is any non-aromatic amino acid
  MISC_FEATURE (7)..(8): Xaas at positions 7 and 8 are independently arginine, lysine or ornithine
XXGSSSXX

SEQ ID NO: 50 - protein, 12 aa, Artificial Sequence
  linker sequence
  MISC_FEATURE (1)..(1): Xaa at position1 is glycine, alanine, valine, leucine, isoleucine, proline, phenylalanine and methionine
  MISC_FEATURE (2)..(2): Xaa at position 2 is any non-aromatic amino acid
  MISC_FEATURE (7)..(8): Xaas at positions 7 and 8 are independently arginine, lysine or ornithine
XXGSSSXXAPQT

SEQ ID NO: 51 - protein, 29 aa, Homo sapiens
SSSSRAPPPSLPSPSRLPGPSDTPILPQK

SEQ ID NO: 52 - protein, 29 aa, Homo sapiens
SSSSKAPPPSLPSPSRLPGPSDTPILPQR

SEQ ID NO: 53 - protein, 8 aa, Artificial Sequence
  linker sequence
  MISC_FEATURE (1)..(1): Xaa at position1 is glycine, alanine, valine, leucine, isoleucine, proline, phenylalanine and methionine
  MISC_FEATURE (2)..(2): Xaa at position 2 is any non-aromatic amino acid
XXGSSSRR

SEQ ID NO: 54 - protein, 12 aa, Artificial Sequence
  Analog of IGF-1 C peptide
  MISC_FEATURE (8)..(8): The Xaa at positions  8 is arginine, lysine or ornithine
GAGSSSRXAPQT

SEQ ID NO: 55 - protein, 12 aa, Artificial Sequence
  linker sequence
  MISC_FEATURE (2)..(2): Xaa at position 2 is any non-aromatic amino acid
  MISC_FEATURE (8)..(8): Xaa at position 8 is arginine, lysine or ornithine
GXGSSSRXAPQT

SEQ ID NO: 56 - protein, 8 aa, Artificial Sequence
  linker sequence
  MISC_FEATURE (2)..(2): Xaa at position 2 is any non-aromatic amino acid
GXGSSSRR

SEQ ID NO: 57 - protein, 9 aa, Artificial Sequence
  linker sequence
GAGSSSRRA

SEQ ID NO: 58 - protein, 10 aa, Artificial Sequence
  linker sequence
GAGSSSRRAP

SEQ ID NO: 59 - protein, 11 aa, Artificial Sequence
  Analog of IGF-1 C peptide
GAGSSSRRAPQ

SEQ ID NO: 60 - protein, 12 aa, Artificial Sequence
  linker sequence
GAGSSSRRAPQT

SEQ ID NO: 61 - protein, 8 aa, Artificial Sequence
  linker sequence
PYGSSSRR

SEQ ID NO: 62 - protein, 8 aa, Artificial Sequence
  linker sequence
PAGSSSRR

SEQ ID NO: 63 - protein, 9 aa, Artificial Sequence
  linker sequence
PAGSSSRRA

SEQ ID NO: 64 - protein, 10 aa, Artificial Sequence
  linker sequence
PAGSSSRRAP

SEQ ID NO: 65 - protein, 11 aa, Artificial Sequence
  linker sequence
PAGSSSRRAPQ

SEQ ID NO: 66 - protein, 12 aa, Artificial Sequence
  linker sequence
PAGSSSRRAPQT

SEQ ID NO: 67 - protein, 28 aa, Homo sapiens
SSSSRAPPPSLPSPSRLPGPSDTPILPQ

SEQ ID NO: 68 - protein, 29 aa, Homo sapiens
  MISC_FEATURE (5)..(5): Xaa at position 5 is lysine or Arginine
  MISC_FEATURE (29)..(29): Xaa at position 29 is lysine or Arginine
SSSSXAPPPSLPSPSRLPGPSDTPILPQX

SEQ ID NO: 69 - protein, 12 aa, Artificial Sequence
  linker sequence
  MISC_FEATURE (7)..(8): Xaas at positions 7 and 8 are independently arginine, lysine or ornithine
GYGSSSXXAPQT

SEQ ID NO: 70 - protein, 4 aa, Homo sapiens
YTPK

SEQ ID NO: 71 - protein, 4 aa, Homo sapiens
FNKP

SEQ ID NO: 72 - protein, 29 aa, Artificial Sequence
  glucagon analogue
  MISC_FEATURE (1)..(1): Xaa at position 1 = His, D-histidine, desaminohistidine, hydroxyl-histidine, acetyl-histidine, homo-histidine or alpha, alpha-dimethyl imidiazole acetic acid (DMIA), N-methyl histidine, alpha-methyl histidine, or imidazole acetic acid
  MISC_FEATURE (2)..(2): Xaa at position 2 = Ser, D-serine, Ala, D-alanine, Val, glycine, n-methyl serine, aminoisobutyric acid (AIB) or N-methyl alanine
  MISC_FEATURE (3)..(3): Xaa at position 3 is Gln, Glu, Orn or  Nle
  MISC_FEATURE (10)..(10): Xaa at position 10 = Tyr, Val or Trp
  MISC_FEATURE (12)..(12): Xaa at position 12 = Ser, Lys or Arg
  MISC_FEATURE (15)..(15): Xaa at position 15 = Asp, Glu, cysteic acid, homoglutamic acid and homocysteic acid
  MISC_FEATURE (16)..(16): Xaa at position 16 = Ser, Gly, Glu, Gln, homoglutamic acid or homocysteic acid
  MISC_FEATURE (17)..(17): Xaa at position 17 = Arg, Gln, Lys, Cys, Orn, homocycstein or acetyl phenylalanine
  MISC_FEATURE (18)..(18): Xaa at position 18 = Arg, Ala,Lys, Cys, Orn, homocycstein or acetyl phenylalanine
  MISC_FEATURE (20)..(20): Xaa at position 20 = Gln, Lys, arginine, ornithine or citrulline
  MISC_FEATURE (21)..(21): Xaa at position 21 = Gln, Glu, Asp, Lys, Cys, Orn, homocycstein or acetyl phenyalanine
  MISC_FEATURE (23)..(23): Xaa at position 23 is Val or Ile
  MISC_FEATURE (24)..(24): Xaa at position 24 = Ala, Gln, Glu, Lys, Cys, Orn, homocycstein or acetyl phenyalanine
  MISC_FEATURE (27)..(27): Xaa at position 27 = Met, Leu, Val or Nle
  MISC_FEATURE (28)..(28): Xaa at position 28 = Asn, Lys or Asp
  MISC_FEATURE (29)..(29): Xaa at position 29 is Thr, Gly, Lys, Cys, Orn, homocycstein or acetyl phenyalanine
XXXGTFTSDXSXYLXXXXAXXFXXWLXXX

SEQ ID NO: 73 - protein, 21 aa, Artificial Sequence
  Analog of Insulin A chain
  MISC_FEATURE (8)..(8): Xaa at position 8 is  threonine or histidine
  MISC_FEATURE (9)..(9): Xaa at position 9 is  valine or tyrosine
  MISC_FEATURE (21)..(21): Xaa at position 21 is asparagine or glycine
GIVEQCCXXICSLYQLENYCX

SEQ ID NO: 74 - protein, 21 aa, Artificial Sequence
  Analog of the insulin A chain
  MISC_FEATURE (8)..(8): Xaa at position 8 is histidine or phenylalnine
  MISC_FEATURE (9)..(9): Xaa at position 9 is arginine, lysine, ornithine or alanine
  MISC_FEATURE (19)..(19): Xaa at position19 is tyrosine, 4-methoxy phenylalanine or 4-amino-phenylalanine
  MISC_FEATURE (21)..(21): Xaa at position 21 is alanine, glycine or asparagine
GIVDECCXXSCDLRRLEMXCX

SEQ ID NO: 75 - protein, 21 aa, Artificial Sequence
  Analog of the insulin B chain
  MISC_FEATURE (1)..(1): Xaa at position 1 is histidine or threonine
  MISC_FEATURE (6)..(6): Xaa at position 6 is histidine, aspartic acid, glutamic acid, homocysteic acid or cysteic acid
  MISC_FEATURE (18)..(18): Xaa at position 18 is alanine, ornithine, lysine or arginine
XLCGAXLVDALYLVCGDXGFY

SEQ ID NO: 76 - protein, 8 aa, Artificial Sequence
  Analog of IGF-1 C peptide
  MISC_FEATURE (7)..(8): The Xaas at positions 7 and 8 are independently arginine, lysine or ornithine
GAGSSSXX

SEQ ID NO: 77 - protein, 12 aa, Artificial Sequence
  Analog of IGF-1 C peptide
  MISC_FEATURE (7)..(8): The Xaas at positions 7 and 8 are independently arginine, lysine or ornithine
GYGSSSXXAPQT

SEQ ID NO: 78 - protein, 10 aa, Artificial Sequence
  peptide fragment representing the carboxy terminal 10 amino acids of Exendin-4
GPSSGAPPPS

SEQ ID NO: 79 - protein, 10 aa, Artificial Sequence
  peptide fragment representing the carboxy terminal 10 amino acids of Exendin-4
  MOD_RES (10)..(10): AMIDATION
GPSSGAPPPS

SEQ ID NO: 80 - protein, 11 aa, Artificial Sequence
  peptide fragment representing the carboxy terminal 10 amino acids of Exendin-4
  MISC_FEATURE (11)..(11): pegylated
GPSSGAPPPSC

SEQ ID NO: 81 - protein, 29 aa, Homo sapiens
  MISC_FEATURE (15)..(15): Xaa at position 15 = Asp, Glu, homoglutamic acid, cysteic acid or homocysteic acid
  MISC_FEATURE (16)..(16): Xaa at position 16 = Ser, Glu, Gln, homoglutamic acid or homocysteic acid
  MISC_FEATURE (20)..(20): Xaa at position 20 = Gln or Lys
  MISC_FEATURE (24)..(24): Xaa at position 24 = Gln or Glu
  MISC_FEATURE (28)..(28): Xaa at position 28 = Asn, Lys or an acidic amino acid
  MISC_FEATURE (29)..(29): Xaa at position 29 = Thr, Gly or an acidic amino acid
HSQGTFTSDYSKYLXXRRAXDFVXWLMXX

SEQ ID NO: 82 - protein, 29 aa, Homo sapiens
  MISC_FEATURE (15)..(15): Xaa at position 15 = Asp, Glu, cysteic acid, homoglutamic acid or homocysteic acid
  MISC_FEATURE (16)..(16): Xaa at position 16 = Ser, Glu, Gln, homoglutamic acid or homocysteic acid
  MISC_FEATURE (20)..(20): Xaa at position 20 = Gln or Lys
  MISC_FEATURE (24)..(24): Xaa at position 24 = Gln or Glu
  MISC_FEATURE (28)..(28): Xaa at position 28 = Asn, Asp or Lys
  MISC_FEATURE (29)..(29): Xaa at position 29 is Thr or Gly
HSQGTFTSDYSKYLDXRRAXDFVXWLMXX

SEQ ID NO: 83 - protein, 29 aa, Artificial Sequence
  glucagon analogue
  MISC_FEATURE (3)..(3): Xaa at position 3= Glu, Orn or Nle
  MISC_FEATURE (15)..(15): Xaa at position 15 = Asp, Glu, homoglutamic acid, cysteic acid or homocysteic acid
  MISC_FEATURE (16)..(16): Xaa at position 16 = Ser, Glu, Gln, homoglutamic acid or homocysteic acid
  MISC_FEATURE (20)..(20): Xaa at position 20 = Gln or Lys
  MISC_FEATURE (24)..(24): Xaa at position 24 = Gln or Glu
  MISC_FEATURE (28)..(28): Xaa at position 28 = Asn, Lys or an acidic amino acid
  MISC_FEATURE (29)..(29): Xaa at position 29 = Thr or an acidic amino acid
HSXGTFTSDYSKYLXXRRAXDFVXWLMXX

SEQ ID NO: 84 - protein, 29 aa, Artificial Sequence
  Glucagon analogue
  MISC_FEATURE (15)..(15): Xaa at position 15 is Glu or Asp
  MISC_FEATURE (28)..(28): Xaa at position 28 is Glu or Asp
  MOD_RES (29)..(29): AMIDATION
HSQGTFTSDYSKYLXERRAQDFVQWLMXT

SEQ ID NO: 85 - protein, 29 aa, Artificial Sequence
  glucagon analogue
  MISC_FEATURE (12)..(16): lactam ring formed between side chain of Lys 12 and Glu 16
  MISC_FEATURE (15)..(15): Xaa at position 15 is Glu or Asp
  MISC_FEATURE (28)..(28): Xaa at position 28 is Glu or Asp
  MOD_RES (29)..(29): AMIDATION
HSQGTFTSDYSKYLXERRAQDFVQWLMXT

SEQ ID NO: 86 - protein, 29 aa, Artificial Sequence
  glucagon analogue
  MISC_FEATURE (15)..(15): Xaa at position 15 is Glu or Asp
  MISC_FEATURE (16)..(20): lactam ring formed between side chain of Glu 16 and Lys 20
  MISC_FEATURE (28)..(28): Xaa at position 28 is Glu or Asp
  MOD_RES (29)..(29): AMIDATION
HSQGTFTSDYSKYLXERRAKDFVQWLMXT

SEQ ID NO: 87 - protein, 29 aa, Artificial Sequence
  glucagon analogue
  MISC_FEATURE (15)..(15): Xaa at position 15 is Glu or Asp
  MISC_FEATURE (20)..(24): lactam ring formed between side chain of Lys 20 and Glu 24
  MISC_FEATURE (28)..(28): Xaa at position 28 is Glu or Asp
  MOD_RES (29)..(29): AMIDATION
HSQGTFTSDYSKYLXSRRAKDFVEWLMXT

SEQ ID NO: 88 - protein, 29 aa, Artificial Sequence
  glucagon analogue
  MISC_FEATURE (15)..(15): Xaa at position 15 is Glu or Asp
  MISC_FEATURE (24)..(28): lactam ring formed between side chain of Glu 24 and Lys 28
  MISC_FEATURE (29)..(29): Xaa at position 29 is Glu or Thr
  MOD_RES (29)..(29): AMIDATION
HSQGTFTSDYSKYLXSRRAQDFVEWLMKX

SEQ ID NO: 89 - protein, 29 aa, Homo sapiens
  MISC_FEATURE (27)..(27): Xaa at position 27 is Met, Leu or Nle
HSQGTFTSDYSKYLDSRRAQDFVQWLXNT

SEQ ID NO: 90 - protein, 29 aa, Artificial Sequence
  glucagon analogue
  MISC_FEATURE (16)..(16): Xaa is Glu, Gln, homoglutamic acid or homocysteic acid
  MISC_FEATURE (17)..(17): Xaa is Arg, Cys, Orn, homocysteine or acetyl phenylalanine
  MISC_FEATURE (27)..(27): Xaa at position 27 is Met, Leu or Nle
HSQGTFTSDYSKYLDXXRAQDFVQWLXNT

SEQ ID NO: 91 - protein, 29 aa, Artificial Sequence
  Glucagon analogue
  MISC_FEATURE (16)..(16): Xaa is Glu, Gln, homoglutamic acid or homocysteic acid
  MISC_FEATURE (21)..(21): Xaa is Asp, Cys, Orn, homocysteine or acetyl phenyalanine
  MISC_FEATURE (27)..(27): Xaa at position 27 is Met, Leu or Nle
HSQGTFTSDYSKYLDXRRAQXFVQWLXNT

SEQ ID NO: 92 - protein, 29 aa, Artificial Sequence
  glucagon analog
  MISC_FEATURE (16)..(16): Xaa is Glu, Gln, homoglutamic acid or homocysteic acid
  MISC_FEATURE (24)..(24): Xaa is Gln, Cys, Orn, homocysteine or acetyl phenylalanine
  MISC_FEATURE (27)..(27): Xaa at postion 27 is Met, Leu or Nle
HSQGTFTSDYSKYLDXRRAQDFVXWLXNT

SEQ ID NO: 93 - protein, 29 aa, Artificial Sequence
  Glucagon analogue
  MISC_FEATURE (16)..(16): Xaa is Glu, Gln, homoglutamic acid or homocysteic acid
  MISC_FEATURE (21)..(21): Xaa is Asp, Cys, Orn, homocysteine or acetyl phenyalanine
  MISC_FEATURE (24)..(24): Xaa is Gln, Cys, Orn, homocysteine or acetyl phenyalanine
  MISC_FEATURE (27)..(27): Xaa at position 27 is Met, Leu or Nle
HSQGTFTSDYSKYLDXRRAQXFVXWLXNT

SEQ ID NO: 94 - protein, 29 aa, Artificial Sequence
  Glucagon analogue
  MISC_FEATURE (21)..(21): Xaa is Asp, Cys, Orn, homocysteine or acetyl phenyalanine
  MISC_FEATURE (27)..(27): Xaa is Met, Leu or Nle
HSQGTFTSDYSKYLDERRAQXFVQWLXNT

SEQ ID NO: 95 - protein, 29 aa, Artificial Sequence
  Glucagon analogue
  MISC_FEATURE (24)..(24): Xaa at position 24 is Gln, Cys, Orn, homocysteine or acetyl phenylalanine
  MISC_FEATURE (27)..(27): Xaa at position 27 is Met, Leu or Nle
HSQGTFTSDYSKYLDERRAQDFVXWLXNT

SEQ ID NO: 96 - protein, 29 aa, Artificial Sequence
  Glucagon analogue
  MISC_FEATURE (16)..(16): Xaa at position 16 is Glu, Gln, homoglutamic acid or homocysteic acid
HSQGTFTSDYSKYLDXRRAQDFVQWLMNT

SEQ ID NO: 97 - protein, 29 aa, Artificial Sequence
  Glucagon analogue
  MISC_FEATURE (27)..(27): Xaa at position 27 = Met, Leu or Nle
HSQGTFTSDYSKYLDERRAQDFVQWLXNT

SEQ ID NO: 98 - protein, 29 aa, Artificial Sequence
  Glucagon analogue
HSQGTFTSDYSKYLDERRAQDFVQWLMNT

SEQ ID NO: 99 - protein, 29 aa, Artificial Sequence
  Glucagon analogue
  MOD_RES (29)..(29): AMIDATION
HSQGTFTSDYSKYLDERRAQDFVQWLMNT

SEQ ID NO: 100 - protein, 29 aa, Artificial Sequence
  glucagon analogue
  MISC_FEATURE (12)..(16): lactam ring formed between side chain of Lys 12 and Glu 16
HSQGTFTSDYSKYLDERRAQDFVQWLMNT

SEQ ID NO: 101 - protein, 29 aa, Artificial Sequence
  glucagon analogue
  MISC_FEATURE (16)..(20): lactam ring formed between side chain of Glu 16 and Lys 20
HSQGTFTSDYSKYLDERRAKDFVQWLMNT

SEQ ID NO: 102 - protein, 29 aa, Artificial Sequence
  glucagon analogue
  MISC_FEATURE (20)..(24): lactam ring formed between side chain of Lys 20 and Glu 24
HSQGTFTSDYSKYLDSRRAKDFVEWLMNT

SEQ ID NO: 103 - protein, 29 aa, Homo sapiens
  MISC_FEATURE (24)..(28): lactam ring formed between side chain of Glu 24 and Lys 28
HSQGTFTSDYSKYLDSRRAQDFVEWLMKT

SEQ ID NO: 104 - protein, 29 aa, Homo sapiens
  MISC_FEATURE (12)..(16): lactam ring formed between side chain of Lys 12 and Glu 16
  MISC_FEATURE (20)..(24): lactam ring formed between side chain of Lys 20 and Glu 24
HSQGTFTSDYSKYLDERRAKDFVEWLMNT

SEQ ID NO: 105 - protein, 29 aa, Homo sapiens
  MISC_FEATURE (16)..(20): lactam ring formed between side chain of Lys 12 and Glu 16
  MISC_FEATURE (24)..(28): lactam ring formed between side chain of Glu 24 and Lys 28
HSQGTFTSDYSKYLDERRADDFVEWLMKT

SEQ ID NO: 106 - protein, 29 aa, Homo sapiens
  MISC_FEATURE (16)..(20): lactam ring formed between side chain of Glu 16 and Lys 20
  MISC_FEATURE (24)..(28): lactam ring formed between side chain of Glu 24 and Lys 28
HSQGTFTSDYSKYLDERRAKDFVEWLMKT

SEQ ID NO: 107 - protein, 30 aa, Artificial Sequence
  Glucagon analogue
  MISC_FEATURE (30)..(30): Xaa = any amino acid
  MOD_RES (30)..(30): AMIDATION
HSQGTFTSDYSKYLDERRAQDFVQWLMNTX

SEQ ID NO: 108 - protein, 29 aa, Homo sapiens
  MISC_FEATURE (16)..(16): Xaa at position 16 = Ser, Glu, Gln, homoglutamic acid or homocysteic acid
  MISC_FEATURE (20)..(20): Xaa at position 20 = glutamine, lysine, arginine, ornithine or citrulline
  MISC_FEATURE (24)..(24): Xaa at position 24 = Gln or Glu
  MISC_FEATURE (28)..(28): Xaa at position 28 = Asn, Asp or Lys
  MISC_FEATURE (29)..(29): Xaa at position 29 = Thr or Gly
HSQGTFTSDYSKYLDXRRAXDFVXWLMXX

SEQ ID NO: 109 - protein, 29 aa, Artificial Sequence
  glucagon analogue
  MISC_FEATURE (12)..(16): lactam ring formed between side chain of Lys 12 and Glu 16
  MISC_FEATURE (28)..(28): Xaa at position 28 is Asp or Asn
  MISC_FEATURE (29)..(29): Xaa at position 29 is Thr or Gly
HSQGTFTSDYSKYLDERRAQDFVQWLMXX

SEQ ID NO: 110 - protein, 29 aa, Artificial Sequence
  glucagon analogue
  MISC_FEATURE (16)..(20): lactam ring formed between side chain of Glu 16 and Lys 20
  MISC_FEATURE (28)..(28): Xaa at position 28 is Asp or Asn
  MISC_FEATURE (29)..(29): Xaa at position 29 is Thr or Gly
HSQGTFTSDYSKYLDERRAKDFVQWLMXX

SEQ ID NO: 111 - protein, 29 aa, Artificial Sequence
  glucagon analogue
  MISC_FEATURE (20)..(24): lactam ring formed between side chain of Lys 20 and Glu 24
  MISC_FEATURE (28)..(28): Xaa at position 28 is Asp or Asn
  MISC_FEATURE (29)..(29): Xaa at position 29 is Thr or Gly
HSQGTFTSDYSKYLDSRRAKDFVEWLMXX

SEQ ID NO: 112 - protein, 29 aa, Artificial Sequence
  glucagon analogue
  MISC_FEATURE (24)..(28): lactam ring formed between side chain of Glu 24 and Lys 28
  MISC_FEATURE (29)..(29): Xaa at position 29 is Thr or Gly
HSQGTFTSDYSKYLDSRRAQDFVEWLMKX

SEQ ID NO: 113 - protein, 29 aa, Homo sapiens
  MISC_FEATURE (16)..(16): Xaa at position 16 = Ser, Glu, Gln, homoglutamic acid or homocysteic acid
  MISC_FEATURE (20)..(20): Xaa at position 20 = glutamine, lysine, arginine, ornithine or citrulline
  MISC_FEATURE (24)..(24): Xaa at position 24 = Gln or Glu
  MISC_FEATURE (28)..(28): Xaa at position 28 = Asn, Asp or Lys
  MISC_FEATURE (29)..(29): Xaa at position 29 = Thr or Gly
HSQGTFTSDYSKYLDXRRAXDFVXWLMXX

SEQ ID NO: 114 - protein, 29 aa, Homo sapiens
HSQGTFTSDYSKYLDERRAKDFVQWLMNT

SEQ ID NO: 115 - protein, 29 aa, Artificial Sequence
  glucagon analogue
HSQGTFTSDYSKYLDSRRAKDFVEWLMNT

SEQ ID NO: 116 - protein, 29 aa, Artificial Sequence
  glucagon analogue
HSQGTFTSDYSKYLDSRRAQDFVEWLMKT

SEQ ID NO: 117 - protein, 8 aa, Artificial Sequence
  peptide fragment representing the carboxy terminal 8 amino acids of oxyntomodulin
KRNRNNIA

SEQ ID NO: 118 - protein, 4 aa, Artificial Sequence
  peptide fragment representing the amino 4 amino acids of oxyntomodulin carboxy terminus of SEQ ID NO 27
KRNR

SEQ ID NO: 119 - protein, 29 aa, homo sapiens
HSQGTFTSDYSKYLDERRAKDFVQWLMNT

SEQ ID NO: 120 - protein, 29 aa, Artificial Sequence
  glucagon analogue
  MISC_FEATURE (1)..(1): Xaa at position 1 = His, D-histidine, desaminohistidine, hydroxyl-histidine, acetyl-histidine, homo-histidine or alpha, alpha-dimethyl imidiazole acetic acid (DMIA), N-methyl histidine, alpha-methyl histidine, or imidazole acetic acid
  MISC_FEATURE (2)..(2): Xaa at position 2 = Ser, D-serine, Ala, D-alanine, Val, glycine, n-methyl serine, aminoisobutyric acid (AIB) or N-methyl alanine
  MISC_FEATURE (3)..(3): Xaa at position 3 is Gln, Glu, Orn or  Nle
  MISC_FEATURE (15)..(15): Xaa at position 15 = Asp, Glu, cysteic acid, homoglutamic acid and homocysteic acid
  MISC_FEATURE (16)..(16): Xaa at position 16 = Ser, Glu, Gln, homoglutamic acid or homocysteic acid
  MISC_FEATURE (20)..(20): Xaa at position 20 = Gln, Lys, arginine, ornithine or citrulline
  MISC_FEATURE (21)..(21): Xaa at position 21 = Gln, Glu, Asp, Lys, Cys, Orn, homocycstein or acetyl phenyalanine
  MISC_FEATURE (23)..(23): Xaa at position 23 is Val or Ile
  MISC_FEATURE (24)..(24): Xaa at position 24 = Ala, Gln, Glu, Lys, Cys, Orn, homocycstein or acetyl phenyalanine
  MISC_FEATURE (27)..(27): Xaa at position 27 = Met, Leu or Nle
  MISC_FEATURE (28)..(28): Xaa at position 28 = Asn, Lys or Asp
  MISC_FEATURE (29)..(29): Xaa at position 29 is Thr or Gly, Lys, Cys, Orn, homocycstein or acetyl phenyalanine
XXXGTFTSDYSKYLXXRRAXXFXXWLXXX

SEQ ID NO: 121 - protein, 38 aa, Artificial Sequence
  glucagon analogue
  MISC_FEATURE (15)..(15): Xaa at position 15 = Asp, Glu, homoglutamic acid, cysteic acid or homocysteic acid
  MISC_FEATURE (16)..(16): Xaa at position 16 = Ser, Glu, Gln, homoglutamic acid or homocysteic acid
  MISC_FEATURE (20)..(20): Xaa at position 20 = Gln or Lys
  MISC_FEATURE (24)..(24): Xaa at position 24 = Gln or Glu
  MISC_FEATURE (28)..(28): Xaa at position 28 = Asn, Lys or Asp
HSQGTFTSDYSKYLXXRRAXDFVXWLMXGGPSSGPPPS

SEQ ID NO: 122 - protein, 29 aa, Artificial Sequence
  glucagon analogue
  MISC_FEATURE (16)..(16): Xaa at position 16 = Ser, Glu, Gln, homoglutamic acid or homocysteic acid
  MISC_FEATURE (20)..(20): Xaa at position 20 = Gln or Lys
  MISC_FEATURE (21)..(21): Xaa at position 21 = Asp, Lys, Cys, Orn, homocycstein or acetyl phenyalanine
  MISC_FEATURE (24)..(24): Xaa at position 24 = Gln, Lys, Cys, Orn, homocycstein or acetyl phenyalanine
  MISC_FEATURE (27)..(27): Xaa at position 27 = Met, Leu or Nle
HSQGTFTSDYSKYLDXRRAXXFVXWLXNT

SEQ ID NO: 123 - protein, 29 aa, Artificial Sequence
  glucagon analogue
  MISC_FEATURE (15)..(15): Xaa at position 15 = Asp, Glu, homoglutamic acid, cysteic acid or homocysteic acid
  MISC_FEATURE (16)..(16): Xaa at position 16 = Ser, Glu, Gln, homoglutamic acid or homocysteic acid
  MISC_FEATURE (20)..(20): Xaa at position 20 = Gln, Lys, Arg, Orn, or citrulline
  MISC_FEATURE (21)..(21): Xaa at position 21 = Asp, Glu, homoglutamic acid, or homocysteic acid
  MISC_FEATURE (24)..(24): Xaa at position 24 = Gln or Glu
  MISC_FEATURE (28)..(28): Xaa at position 28 = Asn, Lys or an acidic amino acid
  MISC_FEATURE (29)..(29): Xaa at position 29 = Thr, Gly or an acidic amino acid
HSQGTFTSDYSKYLXXRRAXXFVXWLMXX

SEQ ID NO: 124 - protein, 29 aa, Homo sapiens
HSQGTFTSDYSKYLDERRAKDFVQWLMNT

SEQ ID NO: 125 - protein, 29 aa, Artificial Sequence
  glucagon analogue
HSQGTFTSDYSKYLDSRRAKDFVEWLMNT

SEQ ID NO: 126 - protein, 29 aa, Artificial Sequence
  glucagon analogue
HSQGTFTSDYSKYLDSRRAQDFVEWLMKT

SEQ ID NO: 127 - protein, 29 aa, Artificial Sequence
  Glucagon analogue
  MISC_FEATURE (2)..(2): Xaa is d-serine, alanine, glycine, n-methyl serine and aminoisobutyric acid
  MOD_RES (29)..(29): AMIDATION
HXQGTFTSDYSKYLDERRAQDFVQWLMNT

SEQ ID NO: 128 - protein, 29 aa, Artificial Sequence
  Glucagon analogue
  MISC_FEATURE (2)..(2): Xaa is aminoisobutyric acid
  MOD_RES (29)..(29): AMIDATION
HXQGTFTSDYSKYLDERRAQDFVQWLMNT

SEQ ID NO: 129 - protein, 39 aa, Artificial Sequence
  Glucagon analogue
  MISC_FEATURE (27)..(27): Xaa is Met, Leu or Nle
HSQGTFTSDYSKYLDERRAQDFVQWLXNTGPSSGAPPPS

SEQ ID NO: 130 - protein, 37 aa, Artificial Sequence
  Glucagon analogue
  MISC_FEATURE (27)..(27): Xaa is Met, Leu or Nle
HSQGTFTSDYSKYLDERRAQDFVQWLXNTKRNRNNIA

SEQ ID NO: 131 - protein, 33 aa, Artificial Sequence
  Glucagon analogue
  MISC_FEATURE (27)..(27): Xaa is Met, Leu or Nle
HSQGTFTSDYSKYLDERRAQDFVQWLXNTKRNR

SEQ ID NO: 132 - protein, 88 aa, Artificial Sequence
  Insulin conjugate
HAEGTFTSDVSSYLEEQAAREFIAWLVRGRGGPEHLCGAHLVDALYLVCGDRGFYFNDRGAGSSSRRGIVDECCHRSCDLRRLENYCN

SEQ ID NO: 133 - protein, 88 aa, Artificial Sequence
  Insulin conjugate
HAEGTFTSDVSSYLEEQAAREFIAWLVRGRGGPEHLCGAHLVDALYLVCGDRGFYFNDRGAGSSSRRGIVDECCHRSCDLRRLENACN

SEQ ID NO: 134 - protein, 88 aa, Artificial Sequence
  Insulin conjugate
HAEGTFTSDVSSYLEEQAAREAIAWLVRGRGGPEHLCGAHLVDALYLVCGDRGFYFNDRGAGSSSRRGIVDECCHRSCDLRRLENYCN

SEQ ID NO: 135 - protein, 86 aa, Artificial Sequence
  Insulin conjugate
HSQGTFTSDYSKYLDSRRAQDFVQWLMNTGPEHLCGAHLVDALYLVCGDRGFYFNDRGAGSSSRRGIVDECCHRSCDLRRLENYCN

SEQ ID NO: 136 - protein, 86 aa, Artificial Sequence
  Insulin conjugate
HSQGTFTSDYSKYLDSRRAQDFVQWLMNTGPEHLCGAHLVDALYLVCGDRGFYFNDRGAGSSSRRGIVDECCHRSCDLRRLENACN

SEQ ID NO: 137 - protein, 86 aa, Artificial Sequence
  Insulin conjugate
HSEGTFTSDYSKYLDSRRAQDFVQWLMNTGPEHLCGAHLVDALYLVCGDRGFYFNDRGAGSSSRRGIVDECCHRSCDLRRLENYCN

SEQ ID NO: 138 - protein, 86 aa, Artificial Sequence
  Insulin conjugate
HSQGTFTSDYSKYLDERRAQDFVQWLMNTGPEHLCGAHLVDALYLVCGDRGFYFNDRGAGSSSRRGIVDECCHRSCDLRRLENYCN

SEQ ID NO: 139 - protein, 90 aa, Artificial Sequence
  Conjugate of GLP-1 and insulin
HAEGTFTSDVSSYLEEQAAREFIAWLVRGRGFVNQHLCGSHLVEALYLVCGERGFFYTPKTGAGSSSRRGIVEQCCTSICSLYQLENYCN

SEQ ID NO: 140 - protein, 88 aa, Artificial Sequence
  Conjugate of glucagon and insulin
HSQGTFTSDYSKYLDSRRAQDFVQWLMNTFVNQHLCGSHLVEALYLVCGERGFFYTPKTGAGSSSRRGIVEQCCTSICSLYQLENYCN

SEQ ID NO: 141 - protein, 35 aa, Homo sapiens
RREAEDLQVGQVELGGGPGAGSLQPLALEGSLQKR

SEQ ID NO: 142 - protein, 23 aa, Artificial Sequence
  Analog of the insulin B chain
  MISC_FEATURE (1)..(1): Xaa at position 1 is histidine or threonine
  MISC_FEATURE (5)..(5): Xaa at position 5 is alanine, glycine or serine
  MISC_FEATURE (6)..(6): Xaa at position 6 is histidine, aspartic acid, glutamic acid, homocysteic acid or cysteic acid
  MISC_FEATURE (9)..(9): Xaa at position 9 is aspartic acid, glutamine or glutamic acid
  MISC_FEATURE (10)..(10): Xaa at position 10 is alanine or threonine
  MISC_FEATURE (17)..(17): Xaa at position 12 is glutamic acid, aspartic acid or asparagine
  MISC_FEATURE (18)..(18): Xaa at position 18 is alanine, ornithine, lysine or arginine
  MISC_FEATURE (21)..(21): Xaa at position 21 is tyrosine or 4-amino-phenylalanine
XLCGXXLVXXLYLVCGXXGFXYT

SEQ ID NO: 143 - protein, 59 aa, Artificial Sequence
  Single chain insulin analog
FVNQHLCGSHLVEALYLVCGERGFFYTPKTGAGSSSRRGIVEQCCTSICSLYQLENYCN

SEQ ID NO: 144 - protein, 57 aa, Artificial Sequence
  Single chain insulin analog
  MISC_FEATURE (4)..(4): Xaa at position 4 is histidine or threonine
  MISC_FEATURE (9)..(9): Xaa at position 9 is ihistidine, aspartic acid, glutamic acid, homocysteic acid or cysteic acid
  MISC_FEATURE (21)..(21): Xaa at position 21 is alanine ornithine or arginine
  MISC_FEATURE (27)..(27): Xaa at position 21 is lysine or aspartic acid
  MISC_FEATURE (28)..(28): Xaa at position 21 is proline, ornithine or arginine
  MISC_FEATURE (44)..(44): Xaa at position 21 is phenylalanine or histidine
GPEXLCGAXLVDALYLVCGDXGFYFNXXGAGSSSRRGIVDECCXRSCDLRRLENYCN

SEQ ID NO: 145 - protein, 57 aa, Artificial Sequence
  Single chain insulin analog
GPEHLCGAHLVDALYLVCGDRGFYFNDRGAGSSSRRGIVDECCHRSCDLRRLENYCN

SEQ ID NO: 146 - protein, 4 aa, Artificial Sequence
  peptide linker
GGGK

SEQ ID NO: 147 - protein, 25 aa, Homo sapiens
  MISC_FEATURE (5)..(5): Xaa at position 5 is histidine or threonine
FVKQXLCGSHLVEALYLVCGERGFF

SEQ ID NO: 148 - protein, 25 aa, Homo sapiens
  MISC_FEATURE (5)..(5): Xaa at position 5 is histidine or threonine
FVNQXLCGSHLVEALYLVCGERGFF

SEQ ID NO: 149 - protein, 5 aa, Artificial Sequence
  peptide fragment of insulin
  MISC_FEATURE (3)..(3): Xaa at position 3 is proline, aspartic acid or glutamic acid
YTXKT

SEQ ID NO: 150 - protein, 5 aa, Artificial Sequence
  peptide fragment of insulin
YTKPT

SEQ ID NO: 151 - protein, 5 aa, Artificial Sequence
  peptide fragment of insulin
YTKPT

SEQ ID NO: 152 - protein, 4 aa, Artificial Sequence
  peptide fragment of insulin
  MISC_FEATURE (3)..(3): Xaa at position 3 is proline, aspartic acid or glutamic acid
YTXK

SEQ ID NO: 153 - protein, 21 aa, Artificial Sequence
  Analog of the insulin A chain
  MISC_FEATURE (8)..(8): Xaa at position 8 is threonine or histidine
  MISC_FEATURE (21)..(21): Xaa at position 21 is asparagine, glycine, alanine, glutamine, glutamate, threonine, or serine
GIVEQCCXSICSLYQLENYCX

SEQ ID NO: 154 - protein, 30 aa, Homo sapiens
  MISC_FEATURE (5)..(5): Xaa at position 5 is histidine or threonine
FVNQXLCGSHLVEALYLVCGERGFFYTEKT

SEQ ID NO: 155 - protein, 30 aa, Homo sapiens
  MISC_FEATURE (5)..(5): Xaa at position 5 is histidine or threonine
FVNQXLCGSHLVEALYLVCGERGFFYTDKT

SEQ ID NO: 156 - protein, 30 aa, Homo sapiens
  MISC_FEATURE (5)..(5): Xaa at position 5 is histidine or threonine
FVNQXLCGSHLVEALYLVCGERGFFYTKPT

SEQ ID NO: 157 - protein, 30 aa, Homo sapiens
  MISC_FEATURE (5)..(5): Xaa at position 5 is histidine or threonine
FVNQXLCGSHLVEALYLVCGERGFFYTPKT

SEQ ID NO: 158 - protein, 29 aa, Homo sapiens
SSSSKAPPPSLPSPSRLPGPSDTPILPQK

SEQ ID NO: 159 - protein, 29 aa, Homo sapiens
SSSSRAPPPSLPSPSRLPGPSDTPILPQK

SEQ ID NO: 160 - protein, 21 aa, Artificial Sequence
  Analog of the insulin A chain
  MISC_FEATURE (21)..(21): Xaa at position 21 is alanine, glycine or asparagine
GIVEQCCHSICSLYQLENYCX

SEQ ID NO: 161 - protein, 30 aa, Homo sapiens; identical to SEQ ID NO: 157
  MISC_FEATURE (5)..(5): Xaa at position 5 is histidine or threonine

SEQ ID NO: 162 - protein, 30 aa, Homo sapiens; identical to SEQ ID NO: 154
  MISC_FEATURE (5)..(5): Xaa at position 5 is histidine or threonine

SEQ ID NO: 163 - protein, 28 aa, Artificial Sequence
  Single chain insulin analog
  MISC_FEATURE (4)..(4): Xaa at position 4 is histidine or threonine
  MISC_FEATURE (9)..(9): Xaa at position 9 is ihistidine, aspartic acid, glutamic acid, homocysteic acid or cysteic acid
  MISC_FEATURE (21)..(21): Xaa at position 21 is alanine ornithine or arginine
  MISC_FEATURE (27)..(27): Xaa at position 21 is lysine or aspartic acid
  MISC_FEATURE (28)..(28): Xaa at position 21 is proline, ornithine or arginine
  MISC_FEATURE (44)..(44): Xaa at position 21 is phenylalanine or histidine
GPEHLCGAXLVDALYLVCGDXGFYFNXX

SEQ ID NO: 164 - protein, 30 aa, Homo sapiens; identical to SEQ ID NO: 155
  MISC_FEATURE (5)..(5): Xaa at position 5 is histidine or threonine

SEQ ID NO: 165 - protein, 30 aa, Homo sapiens; identical to SEQ ID NO: 156
  MISC_FEATURE (5)..(5): Xaa at position 5 is histidine or threonine

SEQ ID NO: 166 - protein, 17 aa, Artificial Sequence
  synthetic insulin receptor antagonist peptide
CGSLDESFYDWFERQLD

SEQ ID NO: 167 - protein, 17 aa, Artificial Sequence
  synthetic insulin receptor antagonist peptide
GSLDESFYDWFERQLDC

SEQ ID NO: 168 - protein, 22 aa, Artificial Sequence
  synthetic insulin receptor antagonist peptide
CSLEEEWAQIQSEVWGRGSPSY

SEQ ID NO: 169 - protein, 22 aa, Artificial Sequence
  synthetic insulin receptor antagonist peptide
SLEEEWAQIQSEVWGRGSPSYC

SEQ ID NO: 170 - protein, 17 aa, Artificial Sequence
  synthetic insulin receptor antagonist peptide
WAQIQSEVWGRGSPSYC

SEQ ID NO: 171 - protein, 23 aa, Artificial Sequence
  synthetic insulin receptor antagonist peptide
ASLEEEWAQIQSEVWGRGSPSYC

SEQ ID NO: 172 - protein, 22 aa, Artificial Sequence
  synthetic insulin receptor antagonist peptide
SAEEEWAQIQSEVWGRGSPSYC

SEQ ID NO: 173 - protein, 22 aa, Artificial Sequence
  synthetic insulin receptor antagonist peptide
SLAEEWAQIQSEVWGRGSPSYC

SEQ ID NO: 174 - protein, 22 aa, Artificial Sequence
  synthetic insulin receptor antagonist peptide
  MISC_FEATURE (2)..(2): Xaa at position 2 is D-Lys
SXEEEWAQIQSEVWGRGSPSYC

SEQ ID NO: 175 - protein, 22 aa, Artificial Sequence
  synthetic insulin receptor antagonist peptide
SIEEEWAQIQSEVWGRGSPSYC

SEQ ID NO: 176 - protein, 22 aa, Artificial Sequence
  synthetic insulin receptor antagonist peptide
SVEEEWAQIQSEVWGRGSPSYC

SEQ ID NO: 177 - protein, 22 aa, Artificial Sequence
  synthetic insulin receptor antagonist peptide
SFEEEWAQIQSEVWGRGSPSYC

SEQ ID NO: 178 - protein, 22 aa, Artificial Sequence
  synthetic insulin receptor antagonist peptide
SWEEEWAQIQSEVWGRGSPSYC

SEQ ID NO: 179 - protein, 22 aa, Artificial Sequence
  synthetic insulin receptor antagonist peptide
SYEEEWAQIQSEVWGRGSPSYC

SEQ ID NO: 180 - protein, 22 aa, Artificial Sequence
  synthetic insulin receptor antagonist peptide
SQEEEWAQIQSEVWGRGSPSYC

SEQ ID NO: 181 - protein, 21 aa, Artificial Sequence
  synthetic insulin receptor antagonist peptide
SLEEEWAQIQSEVWGRGSPSY

SEQ ID NO: 182 - protein, 22 aa, Artificial Sequence
  synthetic insulin receptor antagonist peptide
  MISC_FEATURE (2)..(2): Xaa at position 2 is a hydrophobic amino acid
SXEEEWAQIQSEVWGRGSPSYC

SEQ ID NO: 183 - protein, 16 aa, Artificial Sequence
  synthetic insulin receptor antagonist peptide
GSLDESFYDWFERQLD

(19) United States
(12) Patent Application Publication
(10) Pub. No.: US 2017/0313755 A1
(43) Pub. Date: Nov. 2, 2017

(54) INSULIN RECEPTOR PARTIAL AGONISTS

(71) Applicant: INDIANA UNIVERSITY RESEARCH AND TECHNOLOGY CORPORATION, Indianapolis, IN (US)

(72) Inventors: Richard D. DiMARCHI, Carmel, IN (US); Sarah J. BRANDT, Bloomington, IN (US); Alexander ZAYKOV, Bloomington, IN (US); Yan ZHAO, Albany, CA (US)

(21) Appl. No.: 15/487,768

(22) Filed: Apr. 14, 2017

Related U.S. Application Data
(60) Provisional application No. 62/328,949, filed on Apr. 28, 2016.

(51) Int. Cl. C07K 14/62; C07K 14/00; A61K 38/00

(52) U.S. Cl. CPC C07K 14/62; C07K 14/001; A61K 38/00

(57) ABSTRACT

Disclosed herein are insulin analog conjugates comprising an insulin agonist covalently linked to an insulin antagonist peptide. The conjugates are high potency insulin agonists but with decreased maximal activity relative to the maximal activity of native insulin.

### CROSS REFERENCE TO RELATED APPLICATION

This application claims priority to U.S. Provisional Patent Application No. 62/328,949, filed Apr. 28, 2016, which is incorporated by reference in its entirety into the present application.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: One 96 kilobyte ASCII (Text) file named “263443SeqListing.txt”, created on Apr. 14, 2017.

### BACKGROUND

Insulin is a peptide hormone comprised of a two chain heterodimer that is biosynthetically derived from a low potency single chain proinsulin precursor through enzymatic processing. Human insulin is comprised of two peptide chains (an “A chain” (SEQ ID NO: 1) and “B chain” (SEQ ID NO: 2)) bound together by disulfide bonds and having a total of 51 amino acids. The C-terminal region of the B-chain and the two terminal regions of the A-chain associate in a three-dimensional structure to assemble a site for high affinity binding to the insulin receptor.

Insulin demonstrates unparalleled ability to lower glucose in virtually all forms of diabetes. Unfortunately, its pharmacology is not glucose sensitive and as such it is capable of excessive action that can lead to life-threatening hypoglycemia. Inconsistent pharmacology is a hallmark of insulin therapy such that it is extremely difficult to normalize blood glucose without occurrence of hypoglycemia. Furthermore, native insulin is of short duration of action and requires modification to render it suitable for use in control of basal glucose. Established approaches to delay the onset of insulin action include reduction in solubility, and albumin binding.

The insulin-like growth factors 1 and 2 are single chain liner peptide hormones that are highly homologous in their A and B chain sequences, sharing approximately fifty percent homology with native insulin. The IGF A and B chains are linked by a “C-peptide”, wherein the C-peptides of the two IGFs differ in size and amino acid sequence, the first being twelve and the second being eight amino acids in length. Human IGF-1 is a 70 aa basic peptide having the protein sequence shown in SEQ ID NO: 3, and has a 43% homology with proinsulin (Rinderknecht et al. (1978) J. Biol. Chem. 253:2769-2776). Human IGF-2 is a 67 amino acid basic peptide having the protein sequence shown in SEQ ID NO: 4. The IGFs demonstrate considerably less activity at the insulin B receptor isoform than the A-receptor isoform.

Applicants have previously identified IGF-1 based insulin peptides analogs, (wherein the native Gln-Phe dipeptide of the B-chain is replaced by Tyr-Leu) that display high activity at the insulin receptor (see PCT/US2009/068713, the disclosure of which is incorporated herein). Such analogs (referred to herein as IGF YL analog peptides) are more readily synthesized than insulin and enable the development of co-agonist analogs for insulin and IGF-1 receptors, and selective insulin receptor specific analogs. Furthermore, these insulin analogs can also be formulated as single chain insulin agonists for use in accordance with the present disclosure (see PCT/US2001/040699, the disclosure of which is incorporated herein).

Insulin receptor antagonist peptides have been previously identified through in an in vitro phosphorylation assay. The peptides consist of either a single, or a pair of binding motifs, known to bind to the insulin receptor. As disclosed herein, applicants have discovered novel insulin receptor antagonist peptides that when conjugated to insulin agonists will retain the insulin receptor agonist potency but will have reduced maximal activity relative to the unconjugated insulin agonist. Such conjugates may offer a more precisely controlled onset and duration of insulin action after clearance from the site of administration and equilibration in the plasma.

### SUMMARY

Disclosed herein are high potency insulin agonist conjugates having insulin receptor agonist activity, but reduced maximal activity relative to the maximal activity of the unconjugated insulin agonist. The conjugates disclosed herein comprise an insulin receptor antagonist peptide and an insulin agonist peptide, wherein the antagonist peptide is covalently linked to insulin agonist. In accordance with one embodiment the maximum level of insulin activity of the conjugate can be altered as a function of the insulin antagonist peptide component of the conjugate without substantially impacting the potency of the insulin agonist. Accordingly, by altering the composition of the antagonist peptide bound to the insulin agonist peptide, a set of peptide conjugates can be prepared having similar potencies as the underlying unconjugated insulin agonist peptide, but having varying maximal activities at the insulin receptors. More particularly, the maximal activities are tunable by a single point mutation within the antagonist portion of the insulin/antagonist peptide conjugate. The side chain at position two in the antagonist determines the maximal activity of the conjugate at the insulin receptor in a way that is both predictable and in keeping with current understanding of hydrophobicity and binding.

In accordance with one embodiment of the present disclosure the insulin/antagonist peptide conjugate comprises an insulin receptor agonist peptide having an A chain and B chain peptides, and an insulin antagonist peptide, wherein the antagonist peptide is covalently linked to insulin agonist at the C-terminus of the insulin B chain. In one embodiment the insulin receptor antagonist peptide comprises a sequence of SLEEEWAQIQSEVWGRGSPSY (SEQ ID NO: 181). In one embodiment the insulin receptor antagonist sequence comprises the sequence SX₂EEEWAQIQSEVWGRGSPSYC (SEQ ID NO: 182) wherein X₂ is a hydrophobic amino acid, optionally selected from the group consisting of leucine, isoleucine, d-leucine and valine.

The insulin agonist peptide component of the conjugates of the present invention can be native insulin or any of the known insulin analogs that have activity at the insulin receptor. In one embodiment the insulin agonist peptide comprises an A chain and a B chain, linked together by disulfide bonds, wherein said A chain comprises a sequence of GIVX₄X₅CCX₈X₉X₁₀CX₁₂LX₁₄X₁₅LX₁₇X₁₈YCX₂₁-R₅₃ (SEQ ID NO: 19), and said B chain comprises a sequence of R₆₂-X₂₅LCGX₂₉X₃₀LVX₃₃X₃₄LYLVCGX₄₁X₄₂GFX₄₅ (SEQ ID NO: 20), wherein

X₄ is glutamic acid or aspartic acid;

X₅ is glutamine or glutamic acid

X₈ is histidine, threonine or phenylalanine;

X₉ is serine, arginine, lysine, ornithine or alanine;

X₁₀ is isoleucine or serine;

X₁₂ is serine or aspartic acid;

X₁₄ is tyrosine, arginine, lysine, ornithine or alanine;

X₁₅ is glutamine, glutamic acid, arginine, alanine, lysine, ornithine or leucine;

X₁₇ is glutamic acid, aspartic acid, asparagine, lysine, ornithine or glutamine;

X₁₈ is methionine, asparagine, glutamine, aspartic acid, glutamic acid or threonine;

X₂₁ is selected from the group consisting of alanine, glycine, serine, valine, threonine, isoleucine, leucine, glutamine, glutamic acid, asparagine, aspartic acid, histidine, tryptophan, tyrosine, and methionine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₁ is selected from the group consisting of glutamic acid, aspartic acid or asparagine;

X₄₂ is selected from the group consisting of alanine, ornithine, lysine and arginine;

X₄₅ is tyrosine or phenylalanine;

R₆₂ is selected from the group consisting of AYRPSE (SEQ ID NO: 14), FVNQ (SEQ ID NO: 12), PGPE (SEQ ID NO: 11), a tripeptide glycine-proline-glutamic acid, a tripeptide valine-asparagine-glutamine, a dipeptide proline-glutamic acid, a dipeptide asparagine-glutamine, glutamine, glutamic acid and an N-terminal amine; and

R₅₃ is COOH or CONH₂. In one embodiment the insulin agonist peptide comprises an A and B chain peptides wherein the A chain comprises the sequence GIVEQCCTSICSLYQLENYCN (SEQ ID NO: 1) or GIVDECCRSCDLRRLEMYCA (SEQ ID NO: 5); and

the B chain sequence comprises the sequence GPETLCGAELVDALYLVCGDRGFYFNKPT (SEQ ID NO: 6), FVKQX₂₅LCGSHLVEALYLVCGERGFFYTEKT (SEQ ID NO: 162), FVNQX₂₅LCGSHLVEALYLVCGERGFFYTDKT (SEQ ID NO: 164), FVNQX₂₅LCGSHLVEALYLVCGERGFFYTKPT (SEQ ID NO: 165) or FVNQX₂₅LCGSHLVEALYLVCGERGFFYTPKT (SEQ ID NO: 161) wherein

X₂₅ is selected from the group consisting of histidine and threonine. In one embodiment the C-terminus of the insulin peptide is modified to comprise an activated thiol group, to allow for the covalent linkage of the insulin receptor antagonist peptide. In one embodiment the insulin agonist comprises a Lys at B28 or B29 wherein the Lys side chain is modified to contain an activated thiol group, which reacts with any free sulfhydryl. The activated thiol group can be used to link an antagonist peptide comprising a cysteine residue to the insulin peptide via disulfide bond formation.

In one embodiment, the insulin polypeptide of the insulin/antagonist peptide conjugate further comprises a self-cleaving dipeptide element (U-B) covalently linked to an N-terminal alpha amine or side chain amine of an amino acid of the insulin agonist peptide via an amide or ester linkage (see International applications WO 2009/099763 and PCT/US2009/068713 the disclosures of which are incorporated by reference herein). Subsequent removal of the dipeptide will occur under physiological conditions and in the absence of enzymatic activity. In one embodiment of the dipeptide element (U-B), U is an amino acid or a hydroxy acid, and B is an N-alkylated amino acid linked to said insulin agonist through an amide bond between a carboxyl moiety of B and an amine of the insulin peptide, optionally wherein U, B, or the amino acid of the single chain insulin agonist to which U-B is linked is a non-coded amino acid.

Additional derivatives of the insulin agonist peptides are encompassed by the present disclosure including modifications that improve the solubility of the underlying insulin peptides. In one embodiment the solubility of the insulin agonist peptide is enhanced by the covalent linkage of a hydrophilic moiety to the N-terminus of the A or B chain or to a side chain of an amino acid of one or both of the first and second insulin polypeptides, including the linkage to a side chain of an amino acid of the linking peptide of single chain insulin polypeptides. In one embodiment the hydrophilic moiety is linked to the side chain of an amino acid at a position selected from the group consisting of A9, A14 and A15 of the A chain or positions B1, B2, B10, B22, B28 or B29 of the B chain. In one embodiment the hydrophilic moiety is a polyethylene chain, an acyl group or an alkyl group. In one embodiment the hydrophilic moiety is albumin, including for example, albumins such as human serum albumin (HSA) and recombinant human albumin (rHA). In one embodiment the hydrophilic moiety is a polyethylene glycol (PEG) chain, having a molecular weight selected from the range of about 500 to about 40,000 Daltons. In one embodiment the polyethylene glycol chain has a molecular weight selected from the range of about 500 to about 5,000 Daltons. In another embodiment the polyethylene glycol chain has a molecular weight of about 10,000 to about 20,000 Daltons.

Acylation or alkylation can increase the half-life of the insulin polypeptides in circulation. Acylation or alkylation can advantageously delay the onset of action and/or extend the duration of action at the insulin receptors. The insulin agonist peptide may be acylated or alkylated at the same amino acid position where a hydrophilic moiety is linked, including for example, at an amino acid side chain of the linking moiety in a single chain insulin analog, or on the side chain of an amino acid comprising a self-cleaving dipeptide element.

Also encompassed by the present disclosure are pharmaceutical compositions comprising the insulin/antagonist peptide conjugates disclosed herein, and a pharmaceutically acceptable carrier. In accordance with one embodiment a pharmaceutical composition is provided comprising any of the insulin/antagonist peptide conjugates disclosed herein, preferably at a purity level of at least 90%, 91%, 92%, 93%, 94%, 95%, 96%, 97%, 98% or 99%, and a pharmaceutically acceptable diluent, carrier or excipient. Such compositions may contain a conjugate as disclosed herein at a concentration of at least 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml, 20 mg/ml, 21 mg/ml, 22 mg/ml, 23 mg/ml, 24 mg/ml, 25 mg/ml or higher. In one embodiment the pharmaceutical compositions comprise aqueous solutions that are sterilized and optionally stored within various package containers. In other embodiments the pharmaceutical compositions comprise a lyophilized powder. The pharmaceutical compositions can be further packaged as part of a kit that includes a disposable device for administering the composition to a patient. The containers or kits may be labeled for storage at ambient room temperature or at refrigerated temperature.

In accordance with one embodiment an improved method of regulating blood glucose levels in insulin dependent patients is provided, and more particularly, a method of treating diabetes with a reduced risk of hypoglycemia is provided. The method comprises the steps of administering to a patient an insulin/antagonist peptide conjugate of the present disclosure in an amount therapeutically effective for the control of diabetes.

### BRIEF DESCRIPTION OF THE DRAWINGS

FIG. 1. is a schematic overview of the two step synthetic strategy for preparing human insulin. Details of the procedure are provided in Example 1.

FIG. 2 is a graph comparing insulin receptor specific binding of synthetic human insulin relative to purified native insulin. The synthetic insulin was produced by the approach detailed in FIG. 1 where the A⁷-B⁷ bond is the first disulfide formed. As indicated by the data presented in the graph, the two molecules have similar binding activities.

FIG. 3 is a graph comparing relative insulin receptor binding of native insulin and the IGF1(Y^{B16}L^{B17}) analog. As indicated by the data presented in the graph, the two molecules have similar binding activities.

FIGS. 4A-4D are graphs showing the results of comparative insulin tolerance tests conducted on mice comparing the ability of human insulin to reduce and sustain low blood glucose concentration relative to three different acylated insulin analogs. The polypeptides were tested at two different concentrations (27 nmol/kg and 90 nmol/kg). The acylated insulins included MIU-41, MIU-36 and MIU-37. MIU-41 [B¹(H5,H10,Y16,L17)26A: A¹(H8,rEC16-K14,N18,N21)], is a two chain insulin analog having a C16 acylation via a gamma glutamic acid linker attached to a lysine residue located at position A14. MIU-36 [B¹(C16-K0,H5,H10,Y16,L17)26A: A1(N18,N21)], is a two chain insulin analog having a C16 acylation linked to the N-terminus of the B chain). MIU-37 [B¹(H5,H10,Y16,L17,C16rE-K22)26A: A¹(N18,N21)], is a two chain insulin analog having a C16 acylation via a gamma glutamic acid linker attached to a lysine residue located at position B22.

FIG. 5 is a schematic representation of the reaction scheme used for the chemical modification of the insulin B29 residue to generate an analog with a trityl-protected thiol group.

FIG. 6 is a graph of data from a phosphorylation assay demonstrating the agonism (solid) and antagonism (dashed) of #6-Cys-Insulin (See Table 5).

FIG. 7 is a graph of data from a phosphorylation assay demonstrating the agonism of the #6-Cys-Insulin conjugate and the truncated version, #6(des1-5)-Cys-Insulin.

FIG. 8 is a graph of data from a phosphorylation assay demonstrating the agonism of the #6-Cys-Insulin alanine scans.

FIG. 9 is a graph of data from a phosphorylation assay demonstrating the agonism of #6(L2)-Cys-Insulin (SEQ ID NO:) and #6(I2)-Cys-Insulin.

FIG. 10 is a graph of data from a phosphorylation assay demonstrating the agonism of #6(L2)-Cys-Insulin and #6(dL2)-Cys-Insulin.

FIG. 11 is a graph of data from a phosphorylation assay demonstrating the agonism of #6(L2)-Cys-Insulin and #6(V2)-Cys-Insulin.

FIG. 12 is a graph of data from a phosphorylation assay demonstrating the agonism of #6(L2)-Cys-Insulin and #6(F2)-Cys-Insulin.

FIG. 13 is a graph of data from a phosphorylation assay demonstrating the agonism of #6(L2)-Cys-Insulin and #6(W2)-Cys-Insulin.

FIG. 14 is a graph of data from a phosphorylation assay demonstrating the agonism of #6(L2)-Cys-Insulin and #6(Y2)-Cys-Insulin.

FIG. 15 is a graph of data from a phosphorylation assay demonstrating the decrease in potency but high of maximal activity of #6(Q2)-Cys-Insulin.

FIG. 16 is a graph of data demonstrating the blood glucose concentrations of STZ diabetic mice in response to human insulin, #6(L2)-Cys-Insulin and #6(A2)-Cys-Insulin.

FIG. 17 is a graph demonstrating the maximal activity of #6(X2)-Cys-Insulin Heterodimers at the insulin A (IRA) and insulin B (IRB) subtype receptors, wherein the amino acid at X2 is selected from Leu, Ile, Val, Phe, Trp, Tyr and Ala.

FIG. 18 is a graph demonstrating the results of an insulin tolerance test comparing the activity of the partial insulin agonist S2(L2)-Pen-Insulin conjugates administered at three different concentrations (10, 25 and 50 nmol/kg) relative to native insulin administered at 10 nmol/kg.

### DETAILED DESCRIPTION

### Definitions

In describing and claiming the invention, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “prodrug” is defined as any compound that undergoes chemical modification before exhibiting its pharmacological effects.

As used herein the term “amino acid” encompasses any molecule containing both amino and carboxyl functional groups, wherein the amino and carboxylate groups are attached to the same carbon (the alpha carbon). The alpha carbon optionally may have one or two further organic substituents. For the purposes of the present disclosure designation of an amino acid without specifying its stereochemistry is intended to encompass either the L or D form of the amino acid, or a racemic mixture. However, in the instance where an amino acid is designated by its three letter code and includes a superscript number, the D form of the amino acid is specified by inclusion of a lower case d before the three letter code and superscript number (e.g., dLys⁻¹), wherein the designation lacking the lower case d (e.g., Lys⁻¹) is intended to specify the native L form of the amino acid. In this nomenclature, the inclusion of the superscript number designates the position of the amino acid in the insulin analog sequence, wherein amino acids that are located within the insulin analog sequence are designated by positive superscript numbers numbered consecutively from the N-terminus. Additional amino acids linked to the insulin analog peptide either at the N-terminus or through a side chain are numbered starting with 0 and increasing in negative integer value as they are further removed from the insulin analog sequence. For example, the position of an amino acid within a dipeptide prodrug linked to the N-terminus of an insulin analog is designated aa⁻¹-aa⁰-insulin analog, wherein aa⁰ represents the carboxy terminal amino acid of the dipeptide and aa¹ designates the amino terminal amino acid of the dipeptide.

As used herein the term “hydroxyl acid” encompasses amino acids that have been modified to replace the alpha carbon amino group with a hydroxyl group.

As used herein the term “non-coded amino acid” encompasses any amino acid that is not an L-isomer of any of the following 20 amino acids: Ala, Cys, Asp, Glu, Phe, Gly, His, Ile, Lys, Leu, Met, Asn, Pro, Gln, Arg, Ser, Thr, Val, Trp, Tyr.

A “dipeptide” is a compound formed by linkage of an alpha amino acid or an alpha hydroxyl acid to another amino acid, through a peptide bond.

As used herein the term “chemical cleavage” absent any further designation encompasses a non-enzymatic reaction that results in the breakage of a covalent chemical bond.

A “bioactive polypeptide” refers to polypeptides which are capable of exerting a biological effect in vitro and/or in vivo.

As used herein a general reference to a peptide/polypeptide is intended to encompass peptides/polypeptides that have modified amino and carboxy termini. For example, an amino acid sequence designating the standard amino acids is intended to encompass standard amino acids at the N- and C-terminus as well as a corresponding hydroxyl acid at the N-terminus and/or a corresponding C-terminal amino acid modified to comprise an amide group in place of the terminal carboxylic acid.

As used herein an “acylated” amino acid is an amino acid comprising an acyl group which is non-native to a naturally-occurring amino acid, regardless by the means by which it is produced. Exemplary methods of producing acylated amino acids and acylated peptides are known in the art and include acylating an amino acid before inclusion in the peptide or peptide synthesis followed by chemical acylation of the peptide. In some embodiments, the acyl group causes the peptide to have one or more of (i) a prolonged half-life in circulation, (ii) a delayed onset of action, (iii) an extended duration of action, (iv) an improved resistance to proteases, such as DPP-IV, and (v) increased potency at the IGF and/or insulin peptide receptors.

As used herein, an “alkylated” amino acid is an amino acid comprising an alkyl group which is non-native to a naturally-occurring amino acid, regardless of the means by which it is produced. Exemplary methods of producing alkylated amino acids and alkylated peptides are known in the art and including alkylating an amino acid before inclusion in the peptide or peptide synthesis followed by chemical alkylation of the peptide. Without being held to any particular theory, it is believed that alkylation of peptides will achieve similar, if not the same, effects as acylation of the peptides, e.g., a prolonged half-life in circulation, a delayed onset of action, an extended duration of action, an improved resistance to proteases, such as DPP-IV, and increased potency at the IGF and/or insulin receptors.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein the term “pharmaceutically acceptable salt” encompasses salts of compounds that retain the biological activity of the parent compound, and which are not biologically or otherwise undesirable. Many of the compounds disclosed herein are capable of forming acid and/or base salts by virtue of the presence of amino and/or carboxyl groups or groups similar thereto.

As used herein, the term “hydrophilic moiety” encompasses any compound that is readily water-soluble or readily absorbs water, and which are tolerated in vivo by mammalian species without toxic effects (i.e. are biocompatible). Examples of hydrophilic moieties include polyethylene glycol (PEG), polylactic acid, polyglycolic acid, a polylactic-polyglycolic acid copolymer, polyvinyl alcohol, polyvinylpyrrolidone, polymethoxazoline, polyethyloxazoline, polyhydroxyethyl methacrylate, polyhydroxypropyl methacrylamide, polymethacrylamide, polydimethylacrylamide, and derivatised celluloses such as hydroxymethylcellulose or hydroxyethylcellulose and co-polymers thereof, as well as natural polymers including, for example, albumin, heparin and dextran.

As used herein, the term “treating” includes prophylaxis of the specific disorder or condition, or alleviation of the symptoms associated with a specific disorder or condition and/or preventing or eliminating said symptoms. For example, as used herein the term “treating diabetes” will refer in general to maintaining glucose blood levels near normal levels and may include increasing or decreasing blood glucose levels depending on a given situation.

As used herein an “effective” amount or a “therapeutically effective amount” of an insulin analog refers to a nontoxic but sufficient amount of an insulin analog to provide the desired effect. For example one desired effect would be the prevention or treatment of hyperglycemia.

The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term, “parenteral” means not through the alimentary canal but by some other route such as intranasal, inhalation, subcutaneous, intramuscular, intraspinal, or intravenous.

Throughout the application, all references to a particular amino acid position by letter and number (e.g. position A5) refer to the amino acid at that position of either the A chain (e.g. position A5) or the B chain (e.g. position B5) in the respective native human insulin A chain (SEQ ID NO: 1) or B chain (SEQ ID NO: 2), or the corresponding amino acid position in any analogs thereof. For example, a reference herein to “position B28” absent any further elaboration would mean the corresponding position B27 of the B chain of an insulin analog in which the first amino acid of SEQ ID NO: 2 has been deleted. Similarly, amino acids added to the N-terminus of the native B chain are numbered starting with B0, followed by numbers of increasing negative value (e.g., B-1, B-2 . . . ) as amino acids are added to the N-terminus. Alternatively, any reference to an amino acid position in the linking moiety of a single chain analog, is made in reference to the native C chain of IGF 1 (SEQ ID NO: 17). For example, position 9 of the native C chain (or the “position C9”) has an alanine residue.

As used herein the term “native human insulin peptide” is intended to designate the 51 amino acid heteroduplex comprising the A chain of SEQ ID NO: 1 and the B chain of SEQ ID NO: 2, as well as single-chain insulin analogs that comprise SEQ ID NOS: 1 and 2. The term “insulin polypeptide” as used herein, absent further descriptive language is intended to encompass the 51 amino acid heteroduplex comprising the A chain of SEQ ID NO: 1 and the B chain of SEQ ID NO: 2, as well as single-chain insulin analogs thereof (including for example those disclosed in published international application WO96/34882 and U.S. Pat. No. 6,630,348, the disclosures of which are incorporated herein by reference), including heteroduplexes and single-chain analogs that comprise modified analogs of the native A chain and/or B chain and derivatives thereof (e.g. IGF1 and IGF2) that have activity at the insulin receptors. Such modified analogs include modification of the amino acid at position A19, B16 or B25 to a 4-amino phenylalanine or one or more amino acid substitutions at positions selected from A5, A8, A9, A10, A12, A14, A15, A17, A18, A21, B1, B2, B3, B4, B5, B9, B10, B13, B14, B17, B20, B21, B22, B23, B26, B27, B28, B29 and B30 or deletions of any or all of positions B1-4 and B26-30. Insulin polypeptides as defined herein can also be analogs derived from a naturally occurring insulin by insertion or substitution of a non-peptide moiety, e.g. a retroinverso fragment, or incorporation of non-peptide bonds such as an azapeptide bond (CO substituted by NH) or pseudo-peptide bond (e.g. NH substituted with CH₂) or an ester bond (e.g., a depsipeptide, wherein one or more of the amide (—CONHR—) bonds are replaced by ester (COOR) bonds).

An “A19 insulin analog” is an insulin peptide that has a substitution of 4-amino phenylalanine or 4-methoxy phenylalanine for the native tyrosine residue at position 19 of the A chain of native insulin.

An “IGF1 analog” as used herein is a generic term that encompasses polypeptides that comprise an A and B chain wherein each of the A and B chain sequences share 90% or greater sequence identity with native IGF1 A and B chain sequences, respectively. The term also encompasses IGF YL analogs.

An “IGF2 analog” as used herein is a generic term that encompasses polypeptides that comprise an A and B chain wherein each of the A and B chain sequences share 90% or greater sequence identity with native IGF2 A and B chain sequences, respectively.

An “IGF YL analog” is a peptide comprising an IGF A chain of SEQ ID NO: 19 and an IGF B chain of SEQ ID NO: 51.

As used herein, the term “single-chain insulin analog” encompasses a group of structurally-related proteins wherein insulin or IGF A and B chains, or analogs or derivatives thereof, are covalently linked to one another to form a linear polypeptide chain. As disclosed herein the single-chain insulin analog comprises the covalent linkage of the carboxy terminus of the B chain to the amino terminus of the A chain via a linking moiety.

As used herein the term “insulin A chain”, absent further descriptive language is intended to encompass the 21 amino acid sequence of SEQ ID NO: 1 as well as functional analogs and derivatives thereof, including the A chain of A19 insulin analogs and other analogs known to those skilled in the art, including modification of the sequence of SEQ ID NO: 1 by one or more amino acid insertions, deletions or substitutions at positions selected from A4, A5, A8, A9, A10, A12, A14, A15, A17, A18, A21.

As used herein the term “insulin B chain”, absent further descriptive language is intended to encompass the 30 amino acid sequence of SEQ ID NO: 2, as well as modified functional analogs of the native B chain, including modification of the amino acid at position B16 or B25 to a 4-amino phenylalanine or one or more amino acid insertions, deletions or substitutions at positions selected from B1, B2, B3, B4, B5, B9, B10, B13, B14, B17, B20, B21, B22, B23, B25, B26, B27, B28, B29 and B30 or deletions of any or all of positions B1-4 and B26-30.

As used herein the term “derivative” is intended to encompass chemical modification to a compound (e.g., an amino acid), including chemical modification in vitro, e.g. by introducing a group in a side chain in one or more positions of a polypeptide, e.g. a nitro group in a tyrosine residue, or iodine in a tyrosine residue, or by conversion of a free carboxylic group to an ester group or to an amide group, or by converting an amino group to an amide by acylation, or by acylating a hydroxy group rendering an ester, or by alkylation of a primary amine rendering a secondary amine or linkage of a hydrophilic moiety to an amino acid side chain. Other derivatives are obtained by oxidation or reduction of the side-chains of the amino acid residues in the polypeptide.

As used herein the term IGF A chain, absent further descriptive language is intended to encompass the 21 amino acid sequence of native IGF 1 or IGF 2 (SEQ ID NOs: 5 and 7 respectively), as well as functional analogs thereof known to those skilled in the art, including modification of the sequence of SEQ ID NO: 5 and 7 by one or more amino acid substitutions at positions selected from A5, A8, A9, A10, A12, A14, A15, A17, A18, A21.

As used herein the term “IGF YL B chain”, absent further descriptive language is intended to encompass an amino acid sequence comprising SEQ ID NO: 21, including for example the sequence of SEQ ID NO: 168, as well as analogs of the IGF YL B chain and derivatives thereof, including modification of the amino acid at position B16 or B25 to a 4-amino phenylalanine or one or more amino acid substitutions at positions selected from B1, B2, B3, B4, B5, B9, B10, B13, B14, B17, B20, B21, B22, B23, B26, B27, B28, B29 and B30 or deletions of any or all of positions B1-4 and B26-30.

The term “identity” as used herein relates to the similarity between two or more sequences. Identity is measured by dividing the number of identical residues by the total number of residues and multiplying the product by 100 to achieve a percentage. Thus, two copies of exactly the same sequence have 100% identity, whereas two sequences that have amino acid deletions, additions, or substitutions relative to one another have a lower degree of identity.

Those skilled in the art will recognize that several computer programs, such as those that employ algorithms such as BLAST (Basic Local Alignment Search Tool, Altschul et al. (1993) J. Mol. Biol. 215:403-410) are available for determining sequence identity.

As used herein, the term “selectivity” of a molecule for a first receptor relative to a second receptor refers to the following ratio: EC₅₀ of the molecule at the second receptor divided by the EC₅₀ of the molecule at the first receptor. For example, a molecule that has an EC₅₀ of 1 nM at a first receptor and an EC₅₀ of 100 nM at a second receptor has 100-fold selectivity for the first receptor relative to the second receptor.

As used herein an amino acid “modification” refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids may be purchased from commercial suppliers, synthesized de novo, or chemically modified or derivatized from naturally occurring amino acids.

As used herein an amino acid “substitution” refers to the replacement of one amino acid residue by a different amino acid residue.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln, cysteic acid and homocysteic acid;

III. Polar, positively charged residues:
His, Arg, Lys; Ornithine (Orn)

IV. Large, aliphatic, nonpolar residues:
Met, Leu, Ile, Val, Cys, Norleucine (Nle), homocysteine

V. Large, aromatic residues:
Phe, Tyr, Trp, acetyl phenylalanine.

As used herein the general term “polyethylene glycol chain” or “PEG chain”, encompasses mixtures of condensation polymers of ethylene oxide and water, in a branched or straight chain, represented by the general formula H(OCH₂CH₂)ₙOH, wherein n is at least 2. “Polyethylene glycol chain” or “PEG chain” is used in combination with a numeric suffix to indicate the approximate average molecular weight thereof. For example, PEG-5,000 refers to polyethylene glycol chain having a total molecular weight average of about 5,000 Daltons.

As used herein the term “pegylated” and like terms includes any compound that has been modified from its native state by linking a polyethylene glycol chain to the compound. A “pegylated polypeptide” is a polypeptide that has a PEG chain covalently bound to the polypeptide.

As used herein a “linker” is a bond, molecule or group of molecules that binds two separate entities to one another. Linkers may provide for optimal spacing of the two entities or may further supply a labile linkage that allows the two entities to be separated from each other. Labile linkages include photocleavable groups, acid-labile moieties, base-labile moieties and enzyme-cleavable groups.

As used herein an “insulin dimer” is a complex comprising two insulin polypeptides covalently bound to one another via a linker. The term insulin dimer, when used absent any qualifying language, encompasses both insulin homodimers and insulin heterodimers. An insulin homodimer comprises two identical insulin polypeptides, whereas an insulin heterodimer comprises two insulin polypeptides that differ.

The term “C₁-Cₙ alkyl” wherein n can be from 1 through 6, as used herein, represents a branched or linear alkyl group having from one to the specified number of carbon atoms.

Typical C₁-C₆ alkyl groups include, but are not limited to, methyl, ethyl, n-propyl, iso-propyl, butyl, iso-Butyl, sec-butyl, tert-butyl, pentyl, hexyl and the like.

The terms “C₂-Cₙ alkenyl” wherein n can be from 2 through 6, as used herein, represents an olefinically unsaturated branched or linear group having from 2 to the specified number of carbon atoms and at least one double bond. Examples of such groups include, but are not limited to, 1-propenyl, 2-propenyl (—CH₂—CH═CH₂), 1,3-butadienyl, (—CH═CHCH═CH₂), 1-butenyl (—CH═CHCH₂CH₃), hexenyl, pentenyl, and the like.

The term “C₂-Cₙ alkynyl” wherein n can be from 2 to 6, refers to an unsaturated branched or linear group having from 2 to n carbon atoms and at least one triple bond. Examples of such groups include, but are not limited to, 1-propynyl, 2-propynyl, 1-butynyl, 2-butynyl, 1-pentynyl, and the like.

As used herein the term “aryl” refers to a mono- or bicyclic carbocyclic ring system having one or two aromatic rings including, but not limited to, phenyl, naphthyl, tetrahydronaphthyl, indanyl, indenyl, and the like. The size of the aryl ring and the presence of substituents or linking groups are indicated by designating the number of carbons present. For example, the term “(C₁-C₃ alkyl)(C₆-C₁₀ aryl)” refers to a 5 to 10 membered aryl that is attached to a parent moiety via a one to three membered alkyl chain.

The term “heteroaryl” as used herein refers to a mono- or bi-cyclic ring system containing one or two aromatic rings and containing at least one nitrogen, oxygen, or sulfur atom in an aromatic ring. The size of the heteroaryl ring and the presence of substituents or linking groups are indicated by designating the number of carbons present. For example, the term “(C₁-Cₙ alkyl)(C₅-C₆ heteroaryl)” refers to a 5 or 6 membered heteroaryl that is attached to a parent moiety via a one to “n” membered alkyl chain.

As used herein, the term “halo” refers to one or more members of the group consisting of fluorine, chlorine, bromine, and iodine.

As used herein the term “patient” without further designation is intended to encompass any warm blooded vertebrate domesticated animal (including for example, but not limited to livestock, horses, cats, dogs and other pets) and humans.

The term “isolated” as used herein means having been removed from its natural environment. In some embodiments, the analog is made through recombinant methods and the analog is isolated from the host cell.

The term “purified,” as used herein encompasses the isolation of a molecule or compound in a form that is substantially free of contaminants normally associated with the molecule or compound in a native or natural environment and means having been increased in purity as a result of being separated from other components of the original composition. The term “purified polypeptide” is used herein to describe a polypeptide which has been separated from other compounds including, but not limited to nucleic acid molecules, lipids and carbohydrates.

A “peptidomimetic” refers to a chemical compound having a structure that is different from the general structure of an existing peptide, but that functions in a manner similar to the existing peptide, e.g., by mimicking the biological activity of that peptide. Peptidomimetics typically comprise naturally-occurring amino acids and/or unnatural amino acids, but can also comprise modifications to the peptide backbone. For example a peptidomimetic may include a sequence of naturally-occurring amino acids with the insertion or substitution of a non-peptide moiety, e.g. a retroinverso fragment, or incorporation of non-peptide bonds such as an azapeptide bond (CO substituted by NH) or pseudo-peptide bond (e.g. NH substituted with CH₂), or an ester bond (e.g., depsipeptides, wherein one or more of the amide (—CONHR—) bonds are replaced by ester (COOR) bonds). Alternatively the peptidomimetic may be devoid of any naturally-occurring amino acids.

As used herein the term “charged amino acid” or “charged residue” refers to an amino acid that comprises a side chain that is negatively charged (i.e., de-protonated) or positively charged (i.e., protonated) in aqueous solution at physiological pH. For example, negatively charged amino acids include aspartic acid, glutamic acid, cysteic acid, homocysteic acid, and homoglutamic acid, whereas positively charged amino acids include arginine, lysine and histidine. Charged amino acids include the charged amino acids among the 20 amino acids commonly found in human proteins, as well as atypical or non-naturally occurring amino acids.

As used herein the term “acidic amino acid” refers to an amino acid that comprises a second acidic moiety (other than the alpha carboxylic acid of the amino acid), including for example, a side chain carboxylic acid or sulfonic acid group.

As used herein the term “a mini-PEG linker” absent further descriptive language is a linear polymer of ethylene glycol, comprising 4-16 ethylene glycol units, that covalently links a polypeptide to a second polymer, typically a second polypeptide. Optionally the mini-PEG may comprise amino acids.

### Abbreviations

Insulin analogs will be abbreviated as follows:

The insulin A and B chains will be designated by a capital A for the A chain and a capital B for the B chain wherein a superscript 0 (e.g., A⁰ or B⁰) will designate the base sequence is an insulin sequence (A chain: SEQ ID NO: 1, B chain SEQ ID NO: 2) and a superscript 1 (e.g., A or B¹) will designate the base sequence is an IGF-1 sequence (A chain: SEQ ID NO: 5, B chain SEQ ID NO: 6). Modifications that deviate from the native insulin and IGF sequence are indicated in parenthesis following the designation of the A or B chain (e.g., [B¹(H5,H10,Y16,L17): A¹(H8,N18,N21)]) with the single letter amino acid abbreviation indicating the substitution and the number indicating the position of the substitution in the respective A or B chain, using native insulin numbering. A colon between the A and B chain indicates a two chain insulin whereas a dash will indicate a covalent bond and thus a single chain analog. In single chain analogs a linking moiety will be included between the A and B chains and the designation C¹ refers to the native IGF 1 C peptide, SEQ ID NO: 17. The designation “position C8” in reference to the linking moiety designates an amino acid located at the position corresponding to the eighth amino acid of SEQ ID NO: 17.

### EMBODIMENTS

In an effort to provide a safer form of insulin, applicants have identified insulin receptor antagonist peptides to be used in conjunction with insulin receptor agonists (including native insulin). The combination of these two activities is intended to reduce the maximal activity of the administered insulin agonist peptide and thus reduce the risk of hypoglycemia associated with insulin therapy.

Several potentially antagonistic peptides have been identified using a receptor tyrosine phosphorylation assay in human embryonic kidney (HEK) cells. HEK cells overexpress either the insulin receptor A (IRA) or insulin receptor B (see IRB) isoform, and phosphorylation of the receptors is measured using phosphotyrosine antibodies (See Example 4). Using this assay, applicants have discovered peptides identified with insulin receptor antagonist activity: GSLDESFYDWFERQLG (SEQ ID NO: 183) and SLEEEWAQIQSEVWGRGSPSY (SEQ ID NO: 181).

As disclosed herein these insulin receptor antagonist peptides can be conjugated to insulin receptor agonist peptides to prepare compounds having potent insulin agonist activity while exhibiting a reduced maximal insulin receptor agonist activity relative to the maximal activity of the corresponding unconjugated insulin analog. More particularly, the maximal insulin receptor agonist activity of the conjugates disclosed herein can be engineered base on the selection of the specific insulin antagonist peptide linked to the insulin agonist peptide. Accordingly, by altering the composition of the antagonist peptide of the disclosed conjugates, a set of peptides with similar potencies but varying maximal activities at the insulin receptors can be prepared. More particularly, in one embodiment, the maximal activities are tunable by a single point mutation within the antagonist peptide sequence of the conjugate. For example, the amino acid at position two in the antagonist peptide can be substituted to modify the maximal activity of the conjugate at the insulin receptor in a way that is both predictable and in keeping with current understanding of hydrophobicity and binding.

In accordance with one embodiment of the present disclosure an insulin/antagonists peptide conjugate is provided comprising an insulin agonist peptide having an A chain and B chain peptides, and an insulin receptor antagonist peptide, wherein the antagonist peptide is covalently linked to insulin agonist. The insulin receptor antagonist peptide can be conjugated with the insulin agonist peptide at any convenient site using standard techniques known to those skilled in the art. In one embodiment the insulin receptor antagonist peptide is linked via the N-terminal amine of the insulin A chain or B chain, or at a reactive group of an amino acid side chain of the insulin agonist. In another embodiment the insulin agonist is a single chain insulin agonist and the insulin receptor antagonist peptide is linked to a reactive group of an amino acid side chain of a peptide linker joining the carboxy terminus of the B chain to the amino terminus of the A chain.

In one embodiment the insulin receptor antagonist peptide is lined to the C-terminus of the insulin B chain, optionally through the amino acids side chain of an amino acid at position B28 or B29, relative to the native insulin sequence. The insulin receptor antagonist peptide can be linked either directly or through a linker. In one embodiment the insulin receptor antagonist peptide is linked via the side chain of a Cys or Lys amino acid of the insulin receptor. In one embodiment the insulin receptor antagonist peptide comprises a cysteine and the insulin receptor antagonist peptide is linked to the insulin agonist via a disulfide bond. In one embodiment a lysine present in the carboxy terminus of the B chain (either present at any of positions 25-30 or added as a C-terminal extension) is modified to comprise an activated thiol group that can be used to link an antagonist peptide comprising a cysteine residue to the insulin peptide via disulfide bond formation.

In one embodiment the antagonist peptide comprises a sequence of GSLDESFYDWFERQLG (SEQ ID NO: 183) or SLEEEWAQIQSEVWGRGSPSY (SEQ ID NO: 181). In one embodiment the antagonist peptides are further modified to comprise a cysteine amino acid added at either the N-terminus or the C-terminus to provide a means of conjugating the antagonist peptide to the insulin agonist peptide. In one embodiment the insulin receptor antagonist sequence comprises the sequence SX₂EEEWAQIQSEVWGRGSPSYC (SEQ ID NO: 182) wherein X₂ is a hydrophobic amino acid. In embodiment X₂ is an amino acid selected from the group consisting of Met, Leu, d-leucine, Ile, d-isoleucine, Val, d-valine, Cys, Norleucine (Nle), homocysteine, Phe, Tyr, Trp, or acetyl phenylalanine. In one embodiment X₂ is an amino acid selected from the group consisting of Met, Leu, d-leucine, Ile, d-isoleucine, Val, d-valine, Cys, Norleucine (Nle) and homocysteine. In a further embodiment X₂ is Leu, Ile, Val, or Norleucine (Nle). In embodiment X₂ is selected from the group consisting of leucine, isoleucine, d-leucine and valine. In a further embodiment X₂ is Leu, Ile.

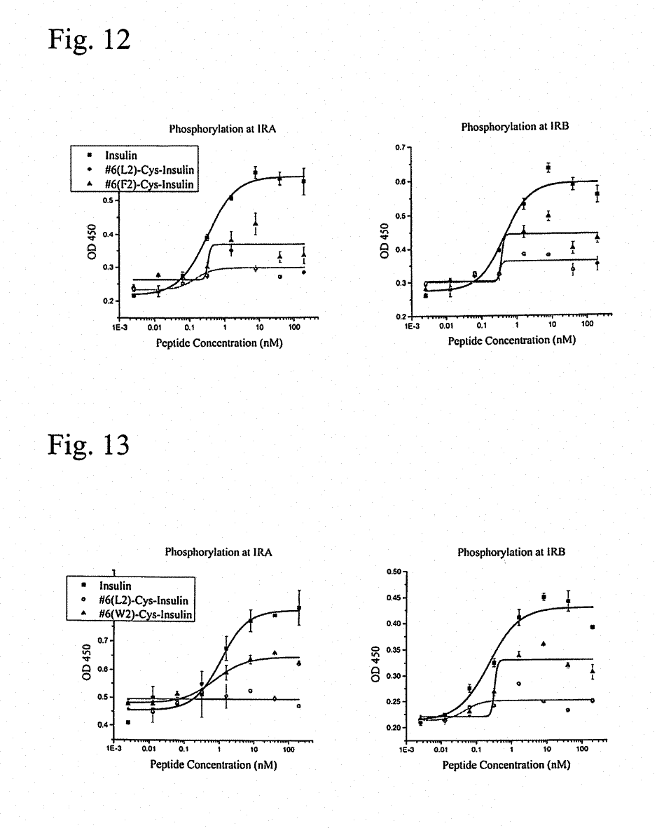

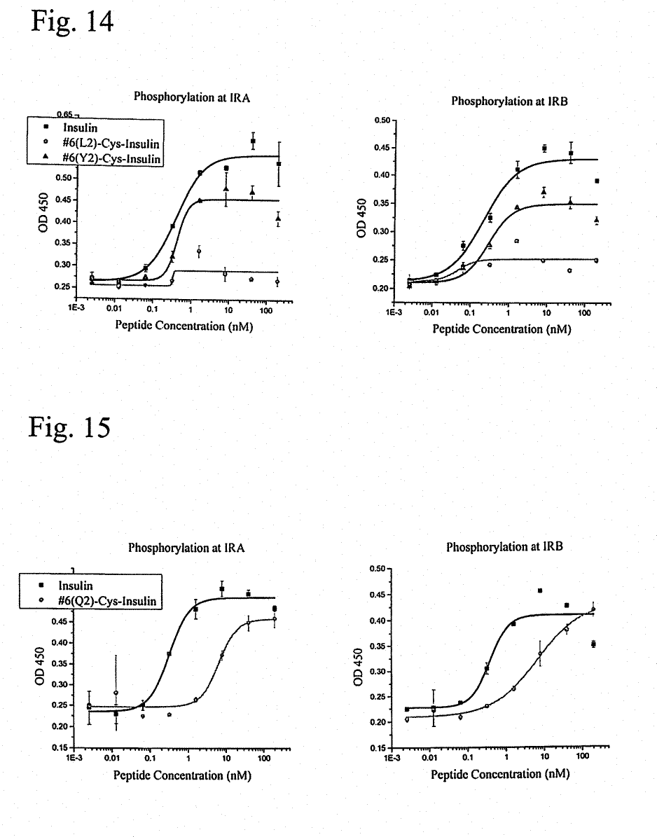

Applicants have discovered that when native insulin is conjugated at position B29 with the insulin antagonist peptide of SEQ ID NO: 182 having either leucine or isoleucine at position 2, the conjugate had very similar, low maximal activities (FIG. 9). However, substituting d-leucine at position two resulted in a low activity conjugate with decreased potency (FIG. 14). This suggests that there is an appropriate size, hydrophobicity and chirality that results in low activity conjugates. Substituting to valine at position two results in a slight increase in maximal activity (FIG. 11). Substituting to phenylalanine at position two results in a further increase in maximal activity, and begins to approach 50% maximal activity at both receptor isoforms (FIG. 12). Substituting to tryptophan at position two results in a conjugate with approximately 60% the maximal activity of native insulin (FIG. 13), and the final substitution to tyrosine, results in a conjugate with approximately 70% the maximal activity of native insulin.

The insulin agonist peptide component of the conjugates of the present invention can be native insulin or any of the known insulin analogs that have activity at the insulin receptor. In one embodiment the insulin agonist peptide comprises an A chain and a B chain wherein said A chain comprises a sequence of GIVX₄X₅CCX₈X₉X₁₀CX₁₂LX₁₄X₁₅LX₁₇X₁₈YCX₂₁-R₅₃ (SEQ ID NO: 19), and said B chain comprises a sequence R₆₂-X₂₅LCGX₂₉X₃₀LVX₃₃X₃₄LYLVCGX₄₁X₄₂GFX₄₅ (SEQ ID NO: 20), wherein

X₄ is glutamic acid or aspartic acid;

X₅ is glutamine or glutamic acid

X₈ is histidine, threonine or phenylalanine;

X₉ is serine, arginine, lysine, ornithine or alanine;

X₁₀ is isoleucine or serine;

X₁₂ is serine or aspartic acid;

X₁₄ is tyrosine, arginine, lysine, ornithine or alanine;

X₁₅ is glutamine, glutamic acid, arginine, alanine, lysine, ornithine or leucine;

X₁₇ is glutamic acid, aspartic acid, asparagine, lysine, ornithine or glutamine;

X₁₈ is methionine, asparagine, glutamine, aspartic acid, glutamic acid or threonine;

X₂₁ is selected from the group consisting of alanine, glycine, serine, valine, threonine, isoleucine, leucine, glutamine, glutamic acid, asparagine, aspartic acid, histidine, tryptophan, tyrosine, and methionine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₁ is selected from the group consisting of glutamic acid, aspartic acid or asparagine;

X₄₂ is selected from the group consisting of alanine, ornithine, lysine and arginine;

X₄₅ is tyrosine or phenylalanine;

R₆₂ is selected from the group consisting of AYRPSE (SEQ ID NO: 14), FVNQ (SEQ ID NO: 12), PGPE (SEQ ID NO: 11), a tripeptide glycine-proline-glutamic acid, a tripeptide valine-asparagine-glutamine, a dipeptide proline-glutamic acid, a dipeptide asparagine-glutamine, glutamine, glutamic acid and an N-terminal amine; and

R₅₃ is COOH or CONH₂.

In one embodiment the insulin peptide is a two chain insulin analog. In another embodiment the insulin peptide is a single chain insulin analog wherein the carboxy terminus of the B chain is linked to the amino terminus of the A chain via a peptide linker. Any of the previous disclosed single chain insulin analogs having activity at the insulin receptor and known to those skilled in the art are encompassed by the present disclosure for conjugation with the insulin antagonist peptides disclosed herein.

In one embodiment the insulin peptide of the conjugate is a two chain insulin wherein the A and B chains are linked by interchain disulfide bonds, wherein the A chain comprises the sequence GIVEQCCX₈X₉ICSLYQLENYCX₂₁-R₅₃ (SEQ ID NO: 73) and the B chain comprises a sequence R₆₂-X₂₅LCGAX₃₀LVDALYLVCGDX₄₂GFY (SEQ ID NO: 75), wherein

X₈ is histidine or threonine;

X₉ is serine, lysine, or alanine;

X₂₁ is alanine, glycine or asparagine;

X₂₅ is histidine or threonine;

X₃₀ is selected from the group consisting of histidine, aspartic acid, glutamic acid, homocysteic acid and cysteic acid;

X₄₂ is selected from the group consisting of alanine ornithine and arginine; and R₅₃ is COOH or CONH₂;

R₆₂ is selected from the group consisting of FVNQ (SEQ ID NO: 12), a tripeptide valine-asparagine-glutamine, a dipeptide asparagine-glutamine, glutamine, and an N-terminal amine; and

R₅₃ is COOH or CONH₂. In one embodiment the A chain comprises the sequence GIVEQCCX₈X₉ICSLYQLENYCX₂₁-R₅₃ (SEQ ID NO: 73) and the B chain comprises the B chain sequence comprises the sequence FVKQX₂₅LCGSHLVEALYLVCGERGFF-R₆₃ (SEQ ID NO: 147), or FVNQX₂₅LCGSHLVEALYLVCGERGFF-R₆₃ (SEQ ID NO: 148), wherein

X₈ is histidine or threonine;

X₉ is serine, lysine, or alanine;

X₂₁ is alanine, glycine or asparagine;

X₂₅ is selected from the group consisting of histidine and threonine; and R₆₃ is selected from the group consisting of YTX₂₈KT (SEQ ID NO: 149), YTKPT (SEQ ID NO: 150), YTX₂₈K (SEQ ID NO: 152), YTKP (SEQ ID NO: 151), YTPK (SEQ ID NO: 70), YTX₂₈, YT, Y and a bond.

In one embodiment the A chain comprises the sequence GIVEQCCX₈SICSLYQLENYCX₂₁-R₅₃ (SEQ ID NO: 153) or GIVEQCCTSICSLYQLENYCN-R₅₃ (SEQ ID NO: 1) and the B chain comprises the sequence FVKQX₂₅LCGSHLVEALYLVCGERGFFYTEKT (SEQ ID NO: 154), FVNQX₂₅LCGSHLVEALYLVCGERGFFYTDKT (SEQ ID NO: 155), FVNQX₂₅LCGSHLVEALYLVCGERGFFYTKPT (SEQ ID NO: 156) or FVNQX₂₅LCGSHLVEALYLVCGERGFFYTPKT (SEQ ID NO: 157) wherein

X₈ is histidine or threonine;

X₂₁ is alanine, glycine or asparagine; X₂₅ is selected from the group consisting of histidine and threonine and R₅₃ is COOH or CONH₂. In one embodiment the A chain comprises a sequence GIVEQCCTSICSLYQLENYCN-R₅₃ (SEQ ID NO: 1) and said B chain comprises a sequence FVNQHLCGSHLVEALYLVCGERGFFYTPKT (SEQ ID NO: 2) wherein R₅₃ is COOH or CONH₂.

In one embodiment the insulin peptide is a single chain insulin analog. In one embodiment the peptide linker joining the B and A chains is selected from the group consisting of SSSSKAPPPSLPSPSRLPGPSDTPILPQR (SEQ ID NO: 158), SSSSRAPPPSLPSPSRLPGPSDTPILPQK (SEQ ID NO: 159), GAGSSSX₅₇X₅₈ (SEQ ID NO: 76), GYGSSSX₅₇X₅₈ (SEQ ID NO: 21) and GYGSSSX₅₇X₅₈APQT; (SEQ ID NO: 77), wherein X₅₇ and X₅₈ are independently arginine, lysine or ornithine. In one embodiment both X₅₇ and X₅₈ are independently arginine. In one embodiment the peptide linking moiety joining the insulin A and B chains to form a single chain insulin analog is a peptide sequence consisting of GYGSSSRR (SEQ ID NO: 18) GAGSSSRR (SEQ ID NO: 22) or GAGSSSRRAPQT (SEQ ID NO: 23).

In one embodiment the insulin agonist peptide comprises A and B chain peptides linked to one another via disulfide bonds, wherein the A chain comprises the sequence GIVEQCCTSICSLYQLENYCN (SEQ ID NO: 1) or GIVDECCRSCDLRRLEMYCA (SEQ ID NO: 5); and

the B chain sequence comprises the sequence GPETLCGAELVDALYLVCGDRGFYFNKPT (SEQ ID NO: 6), FVKQX₂₅LCGSHLVEALYLVCGERGFFYTEKT (SEQ ID NO: 162), FVNQX₂₅LCGSHLVEALYLVCGERGFFYTDKT (SEQ ID NO: 164), FVNQX₂₅LCGSHLVEALYLVCGERGFFYTKPT (SEQ ID NO: 165) or FVNQX₂₅LCGSHLVEALYLVCGERGFFYTPKT (SEQ ID NO: 161) wherein X₂₅ is selected from the group consisting of histidine and threonine. In one embodiment the C-terminus of the insulin peptide is modified to comprise an activated thiol group, to allow for the covalent linkage of the insulin receptor antagonist peptide. In one embodiment the insulin agonist is modified at the B28 or B29 residue to contain an activated thiol group, which reacts with any free sulfhydryl. Alternatively one or more amino acids can be added to the C-terminus of the B chain, wherein the C-terminal addition comprises an amino acid having an activated thiol group. The activated thiol group can be used to link an antagonist peptide comprising a cysteine residue to the insulin peptide via disulfide bond formation.

In one embodiment an insulin/antagonist peptide conjugate is provided comprising a native insulin comprising an A chain of SEQ ID NO: 1 and a B chain of SEQ ID NO: 2 conjugated to an insulin receptor antagonist peptide of SEQ ID NO: 182 wherein X₂ is selected form the group consisting of Leu, Ile, d-Leu or Val. Applicants have discovered that this conjugate, having the antagonist peptide linked through a C-terminal cysteine (optionally at B29), displays only 20-30% of the maximal activity of native insulin. An alanine scan of the antagonist peptide of SEQ ID NO: 169 revealed that the amino acid residue at position two is a key regulator of the maximal activity of the conjugate. Mutating this amino acid residue resulted in a series of peptides with similar EC50 values, but varying levels of maximal activity. It was found that the maximal activity correlated with hydrophobicity of the side chain at position two.

### Structure of Insulin Peptide Agonist for Use in the Disclosed Conjugates

In some embodiments, the insulin peptide of the presently disclosed conjugates is native insulin, comprising the A chain of SEQ ID NO: 1 and the B chain of SEQ ID NO: 2, or an analog of native insulin, including for example a single-chain insulin analog comprising SEQ ID NOS: 1 and 2. In accordance with the present disclosure analogs of insulin encompass polypeptides comprising an A chain and a B chain wherein the insulin analogs differ from native insulin by one or more amino acid substitutions at positions selected from A5, A8, A9, A10, A12, A14, A15, A17, A18, A21, B1, B2, B3, B4, B5, B9, B10, B13, B14, B17, B20, B21, B22, B23, B26, B27, B28, B29 and B30 or deletions of any or all of positions B1-4 and B26-30.

In one embodiment the insulin peptide is an insulin analog wherein:

(a) the amino acid residue at position B28 is substituted with Asp, Lys, Leu, Val, or Ala, and the amino acyl residue at position B29 is Lys or Pro;

(b) the amino acid residues at any of positions B27, B28, B29, and B30 are deleted or substituted with a nonnative amino acid. In one embodiment an insulin analog is provided comprising an Asp substituted at position B28 or a Lys substituted at position 28 and a proline substituted at position B29. Additional insulin analogs are disclosed in Chance, et al., U.S. Pat. No. 5,514,646; Chance, et al., U.S. patent application Ser. No. 08/255,297; Brems, et al., Protein Engineering, 5:527-533 (1992); Brange, et al., EPO Publication No. 214,826 (published Mar. 18, 1987); and Brange, et al., Current Opinion in Structural Biology, 1:934-940 (1991). The disclosures of which are expressly incorporated herein by reference.

Insulin analogs may also have replacements of the amidated amino acids with acidic forms. For example, Asn may be replaced with Asp or Glu. Likewise, Gln may be replaced with Asp or Glu. In particular, Asn(A18), Asn(A21), or Asp(B3), or any combination of those residues, may be replaced by Asp or Glu. Also, Gln(A15) or Gln(B4), or both, may be replaced by either Asp or Glu.

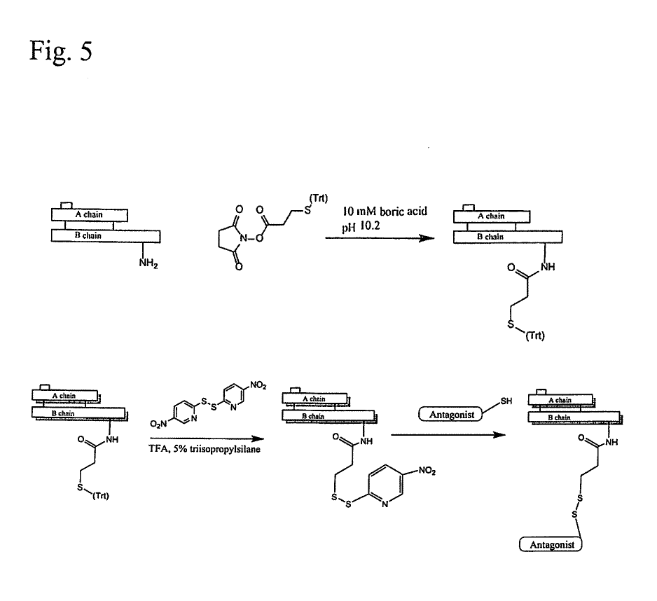

As disclosed herein single chain insulin agonists are provided comprising a B chain and an A chain of human insulin, or analogs or derivative thereof, wherein the carboxy terminus of the B chain is linked to the amino terminus of the A chain via a linking moiety. In one embodiment the A chain is an amino acid sequence selected from the group consisting of GIVEQCCTSICSLYQLENYCN (SEQ ID NO: 1), GIVDECCFRSCDLRRLEMYCA (SEQ ID NO: 5) or GIVEECCFRSCDLALLETYCA (SEQ ID NO: 7) and the B chain comprises the sequence FVNQHLCGSHLVEALYLVCGERGFFYTPKT (SEQ ID NO: 2), GPETLCGAELVDALYLVCGDRGFYFNKPT (SEQ ID NO: 6) or AYRPSETLCGGELVDTLYLVCGDRGFYFSRPA (SEQ ID NO: 8), or a carboxy shortened sequence thereof having one to five amino acids corresponding to B26, B27, B28, B29 and B30 deleted, and analogs of those sequences wherein each sequence is modified to comprise one to five amino acid substitutions at positions corresponding to native insulin positions (see peptide alignment shown in FIG. 5) selected from A5, A8, A9, A10, A14, A15, A17, A18, A21, B1, B2, B3, B4, B5, B9, B10, B13, B14, B20, B22, B23, B26, B27, B28, B29 and B30. In one embodiment the amino acid substitutions are conservative amino acid substitutions. Suitable amino acid substitutions at these positions that do not adversely impact insulin's desired activities are known to those skilled in the art, as demonstrated, for example, in Mayer, et al., Insulin Structure and Function, Biopolymers. 2007; 88(5):687-713, the disclosure of which is incorporated herein by reference.

Additional amino acid sequences can be added to the amino terminus of the B chain or to the carboxy terminus of the A chain of the single chain insulin agonists of the present invention. For example, a series of negatively charged amino acids can be added to the amino terminus of the B chain, including for example a peptide of 1 to 12, 1 to 10, 1 to 8 or 1 to 6 amino acids in length and comprising one or more negatively charged amino acids including for example glutamic acid and aspartic acid. In one embodiment the B chain amino terminal extension comprises 1 to 6 charged amino acids. In one embodiment the B chain amino terminal extension comprises the sequence GX₆₁X₆₂X₆₃X₆₄X₆₅K (SEQ ID NO: 26) or X₆₁X₆₂X₆₃X₆₄X₆₅RK (SEQ ID NO: 27), wherein X₆₁, X₆₂, X₆₃ X₆₄ and X₆₅ are independently glutamic acid or aspartic acid. In one embodiment the B chain comprises the sequence GEEEEEKGPEHLCGAHLVDALYLVCGDX₄₂GFY (SEQ ID NO: 28), wherein X₄₂ is selected from the group consisting of alanine lysine, ornithine and arginine.

High potency insulin/antagonist peptide conjugates can also be prepared based on using a modified IGF I and IGF II sequence described in published International application no. WO 2010/080607, the disclosure of which is expressly incorporated herein by reference, as the insulin peptide component. More particularly, analogs of IGF I and IGF II that comprise a substitution of a tyrosine leucine dipeptide for the native IGF amino acids at positions corresponding to B16 and B17 of native insulin have a tenfold increase in potency at the insulin receptor.

In accordance with one embodiment the insulin peptide for use in the present disclosure comprises a B chain sequence of R₆₂-X₂₅LCGX₂₉X₃₀LVX₃₃X₃₄LYLVCGX₄₁X₄₂GFX₄₅ (SEQ ID NO: 20) and an A chain sequence of GIVX₄X₅CCX₈X₉X₁₀CX₁₂LX₁₄X₁₅LX₁₇X₁₈X₁₉CX₂₁-R₅₃ (SEQ ID NO: 29) wherein

X₄ is glutamic acid or aspartic acid;

X₅ is glutamine or glutamic acid X₈ is histidine, threonine or phenylalanine;

X₉ is serine, arginine, lysine, ornithine or alanine;

X₁₀ is isoleucine or serine;

X₁₂ is serine or aspartic acid X₁₄ is tyrosine, arginine, lysine, ornithine or alanine;

X₁₅ is glutamine, glutamic acid, arginine, alanine, lysine, ornithine or leucine;

X₁₇ is glutamine, glutamic acid, arginine, aspartic acid or lysine, ornithine

X₁₈ is methionine, asparagine, glutamine, aspartic acid, glutamic acid or threonine;

X₁₉ is tyrosine, 4-methoxy-phenylalanine or 4-amino phenylalanine;

X₂₁ is selected from the group consisting of alanine, glycine, serine, valine, threonine, isoleucine, leucine, glutamine, glutamic acid, asparagine, aspartic acid, histidine, tryptophan, tyrosine, and methionine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid, glutamine and glutamic acid;

X₃₄ is selected from the group consisting of alanine and threonine;

X₄₁ is selected from the group consisting of glutamic acid, aspartic acid or asparagine;

X₄₂ is selected from the group consisting of alanine, lysine, ornithine and arginine;

X₄₅ is tyrosine, histidine, asparagine or phenylalanine;

R₆₂ is selected from the group consisting of AYRPSE (SEQ ID NO: 14), FVNQ (SEQ ID NO: 12), PGPE (SEQ ID NO: 11), a tripeptide glycine-proline-glutamic acid, a tripeptide valine-asparagine-glutamine, a dipeptide proline-glutamic acid, a dipeptide asparagine-glutamine, glutamine, glutamic acid and a bond; and R₅₃ is COOH or CONH₂. In one embodiment the A chain and the B chain are linked to one another by interchain disulfide bonds, including those that form between the A and B chains of native insulin. In an alternative embodiment the A and B chains are linked together as a linear single chain-insulin peptide.

In one embodiment the conjugates comprise an insulin peptide wherein the A chain comprises a sequence of GIVEQCCXISICSLYQLENX₂CX₃ (SEQ ID NO: 30) and said B chain sequence comprises a sequence of X₄LCGX₅X₆LVEALYLVCGERGFF (SEQ ID NO: 31), wherein

X₁ is selected from the group consisting of threonine and histidine;

X₂ is tyrosine, 4-methoxy-phenylalanine or 4-amino phenylalanine;

X₃ is selected from the group consisting of asparagine and glycine;

X₄ is selected from the group consisting of histidine and threonine;

X₅ is selected from the group consisting of alanine, glycine and serine;

X₆ is selected from the group consisting of histidine, aspartic acid, glutamic acid, homocysteic acid and cysteic acid.

In accordance with one embodiment an insulin analog is provided wherein the A chain of the insulin peptide comprises the sequence GIVEQCCX₈X₉ICSLYQLENYCX₂₁-R₅₃ (SEQ ID NO: 73) or GIVEQCCX₈SICSLYQLX₁₇NYCX₂₁ (SEQ ID NO: 32) and the B chain comprising the sequence R₆₂-X₂₅LCGX₂₉X₃₀LVX₃₃X₃₄LYLVCGX₄₁X₄₂GFX₄₅YT-Z₁-B₁ (SEQ ID NO: 142), wherein

X₅ is selected from the group consisting of threonine and histidine;

X₉ is valine or tyrosine;

X₁₇ is glutamine or glutamic acid;

X₂₁ is asparagine or glycine;

X₂₅ is histidine or threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₁ is selected from the group consisting of glutamic acid, aspartic acid or asparagine;

X₄₂ is selected from the group consisting of alanine, ornithine, lysine and arginine;

X₄₅ is tyrosine or phenylalanine;

R₆₂ is selected from the group consisting of FVNQ (SEQ ID NO: 12), a tripeptide valine-asparagine-glutamine, a dipeptide asparagine-glutamine, glutamine and an N-terminal amine

Z₁ is a dipeptide selected from the group consisting of aspartate-lysine, lysine-proline, and proline-lysine; and

B₁ is selected from the group consisting of threonine, alanine or a threonine-arginine-arginine tripeptide.

In accordance with one embodiment an insulin analog is provided wherein the A chain of the insulin peptide comprises the sequence GIVEQCCX₈SICSLYQLX₁₇NX₁₉CX₂₁ (SEQ ID NO: 32) and the B chain comprising the sequence X₂₅LCGX₂₉X₃₀LVEALYLVCGERGFF (SEQ ID NO: 33) wherein

X₈ is selected from the group consisting of threonine and histidine;

X₁₇ is glutamic acid or glutamine;

X₁₉ is tyrosine, 4-methoxy-phenylalanine or 4-amino phenylalanine;

X₂₁ is asparagine or glycine;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid. In a further embodiment the B chain comprises the sequence X₂₂VNQX₂₅LCGX₂₉X₃₀LVEALYLVCGERGFFYT-Z₁-B₁ (SEQ ID NO: 34) wherein

X₂₂ is selected from the group consisting of phenylalanine and desamino-phenylalanine;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid; Z₁ is a dipeptide selected from the group consisting of aspartate-lysine, lysine-proline, and proline-lysine; and B1 is selected from the group consisting of threonine, alanine or a threonine-arginine-arginine tripeptide.

In accordance with some embodiments the A chain comprises the sequence GIVEQCCX₈SICSLYQLX₁₇NX₁₉CX₂₃ (SEQ ID NO: 32) or GIVDECCX₈X₉SCDLX₁₄X₁₅LX₁₇X₁₈ X₁₉CX₂₁-R₅₃ (SEQ ID NO: 35), and the B chain comprises the sequence X₂₅LCGX₂₉X₃₀LVX₃₃X₃₄LYLVCGDX₄₂GFX₄₅ (SEQ ID NO: 36) wherein

X₈ is histidine or phenylalanine;

X₉ and X₁₄ are independently selected from arginine, lysine, ornithine or alanine;

X₁₅ is arginine, lysine, ornithine or leucine;

X₁₇ is glutamic acid or glutamine;

X₁₈ is methionine, asparagine or threonine;

X₁₉ is tyrosine, 4-methoxy-phenylalanine or 4-amino phenylalanine;

X₂₁ is alanine, glycine or asparagine;

X₂₃ is asparagine or glycine;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₂ is selected from the group consisting of alanine, lysine, ornithine and arginine;

X₄₅ is tyrosine; and R₅₃ is COOH or CONH₂.

In a further embodiment the A chain comprises the sequence GIVDECCX₈X₉SCDLX₁₄X₁₅LX₁₇X₁₈ X₁₉CX₂₁-R₅₃ (SEQ ID NO: 35), and the B chain comprises the sequence X₂₅LCGX₂₉X₃₀LVX₃₃X₃₄LYLVCGDX₄₂GFX₄₅ (SEQ ID NO: 36) wherein

X₈ is histidine;

X₉ and X₁₄ are independently selected from arginine, lysine, ornithine or alanine;

X₁₅ is arginine, lysine, ornithine or leucine;

X₁₇ is glutamic acid, aspartic acid, asparagine, lysine, ornithine or glutamine;

X₁₈ is methionine, asparagine or threonine;

X₁₉ is tyrosine, 4-methoxy-phenylalanine or 4-amino phenylalanine;

X₂₁ is alanine, glycine or asparagine;

X₂₃ is asparagine or glycine;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₂ is selected from the group consisting of alanine, lysine, ornithine and arginine;

X₄₅ is tyrosine or phenylalanine and

R₅₃ is COOH or CONH₂. In a further embodiment the A chain comprises the sequence GIVDECCHX₉SCDLX₁₄X₁₅LX₁₇MX₁₉CX₂₁-R₅₃ (SEQ ID NO: 37), and the B chain comprises the sequence X₂₅LCGAX₃₀LVDALYLVCGDX₄₂GFX₄₅ (SEQ ID NO: 38) wherein

X₉, X₁₄ and X₁₅ are independently ornithine, lysine or arginine;

X₁₇ is glutamic acid or glutamine;

X₁₉ is tyrosine, 4-methoxy-phenylalanine or 4-amino phenylalanine;

X₂₁ is alanine, glycine or asparagine;

X₂₅ is selected from the group consisting of histidine and threonine;

X₃₀ is selected from the group consisting of histidine, aspartic acid and glutamic acid;

X₄₂ is selected from the group consisting of alanine, lysine, ornithine and arginine;

X₄₅ is tyrosine or phenylalanine and

R₅₃ is COOH or CONH₂. In one embodiment the B chain is selected from the group consisting of HLCGAELVDALYLVCGDX₄₂GFY (SEQ ID NO: 39), GPEHLCGAELVDALYLVCGDX₄₂GFY (SEQ ID NO: 40), GPEHLCGAELVDALYLVCGDX₄₂GFYFNPKT (SEQ ID NO: 41) and GPEHLCGAELVDALYLVCGDX₄₂GFYFNKPT (SEQ ID NO: 42), wherein X₄₂ is selected from the group consisting of ornithine, lysine and arginine. In a further embodiment the A chain comprises the sequence GIVDECCHX₉SCDLX₁₄X₁₅LQMYCN-R₅₃ (SEQ ID NO: 43), wherein X₉, X₁₄ and X₁₅ are independently ornithine, lysine or arginine.

In another embodiment, the A chain comprises the sequence GIVEQCCHSICSLYQLENYCX₂₁-R₅₃ (SEQ ID NO: 160) and the B chain comprises the sequence FVKQX₂₅LCGSHLVEALYLVCGERGFF-R₆₃ (SEQ ID NO: 147), or FVNQX₂₅LCGSHLVEALYLVCGERGFF-R₆₃ (SEQ ID NO: 148), wherein

X₂₁ is alanine, glycine or asparagine; and

X₂₅ is selected from the group consisting of histidine and threonine;

X₂₈ is proline, aspartic acid or glutamic acid; and

R₆₃ is selected from the group consisting of YTX₂₈KT (SEQ ID NO: 149), YTKPT (SEQ ID NO: 150), YTX₂₈K (SEQ ID NO: 152), YTKP (SEQ ID NO: 151), YTPK (SEQ ID NO: 70), YTX₂₈, YT, Y and a bond. In one embodiment the B chain comprises the sequence FVKQX₂₅LCGSHLVEALYLVCGERGFFYTEKT (SEQ ID NO: 162), FVNQX₂₅LCGSHLVEALYLVCGERGFFYTDKT (SEQ ID NO: 164), FVNQX₂₅LCGSHLVEALYLVCGERGFFYTKPT (SEQ ID NO: 165) or FVNQX₂₅LCGSHLVEALYLVCGERGFFYTPKT (SEQ ID NO: 161) wherein
X₂₅ is selected from the group consisting of histidine and threonine.

### Single Chain Insulin Peptide Agonists

As disclosed herein linking moieties can be used to link human insulin A and B chains, or analogs or derivatives thereof, wherein the carboxy terminus of the B25 amino acid of the B chain is directly linked to a first end of a linking moiety, wherein the second end of the linking moiety is directly linked to the amino terminus of the A1 amino acid of the A chain via the intervening linking moiety.

In accordance with one embodiment the insulin peptide is a single chain insulin agonist that comprises the general structure B-LM-A wherein B represents an insulin B chain, A represents an insulin A chain, and LM represents a linking moiety linking the carboxy terminus of the B chain to the amino terminus of the A chain. Suitable linking moieties for joining the B chain to the A chain are disclosed herein under the header Linking Moieties for Single Chain-Insulin Analogs and the respective subheaders “Peptide linkers”. In one embodiment the linking moiety comprises a linking peptide, and more particularly, in one embodiment the peptide represents an analog of the IGF-1 C peptide. Additional exemplary peptide linkers include but are not limited to the sequence X₅₁X₅₂GSSSX₅₇X₅₈ (SEQ ID NO: 49) or X₅₁X₅₂GSSSX₅₇X₅₈APQT (SEQ ID NO: 50) wherein X₅₁ is selected from the group consisting of glycine, alanine, valine, leucine, isoleucine and proline, X₅₂ is alanine, valine, leucine, isoleucine or proline and X₅₇ or X₅₈ are independently arginine, lysine, cysteine, homocysteine, acetyl-phenylalanine or ornithine, optionally with a hydrophilic moiety linked to the side chain of the amino acid at position 7 or 8 of the linking moiety (i.e., at the X₅₇ or X₅₈ position). Amino acid positions of the linking moiety are designated based on the corresponding position in the native C chain of IGF 1 (SEQ ID NO: 17). In another embodiment the peptide linking moiety comprises a 29 contiguous amino acid sequence having greater than 70%, 80%, 90% sequence identity to SSSSX₅₀APPPSLPSPSRLPGPSDTPILPQX₅₁ (SEQ ID NO: 68), wherein X₅₀ and X₅₁ are independently selected from arginine and lysine. In one embodiment the linking moiety is a non-peptide linker comprising a relatively short bifunctional non-peptide polymer linker that approximates the length of an 8-16 amino acid sequence. In one embodiment the non-peptide linker has the structure:

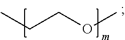

wherein m is an integer ranging from 10 to 14 and the linking moiety is linked directly to the B25 amino acid of the B chain. In accordance with one embodiment the non-peptide linking moiety is a polyethylene glycol linker of approximately 4 to 20, 8 to 18, 8 to 16, 8 to 14, 8 to 12, 10 to 14, 10 to 12 or 11 to 13 monomers.

In one embodiment an insulin/antagonist peptide conjugate is provided that comprises an insulin peptide having the structure: IB-LM-IA, wherein IB comprises the sequence R₆₂-X₂₅LCGX₂₉X₃₀LVX₃₃X₃₄LYLVCGX₄₁X₄₂GFX₄₅ (SEQ ID NO: 20), LM is a linking moiety as disclosed herein that covalently links IB to IA, and IA comprises the sequence GIVX₄X₅CCX₈X₉X₁₀CX₁₂LX₁₄X₁₅LX₁₇X₁₈X₁₉CX₂₁-R₅₃ (SEQ ID NO: 29), wherein

X₄ is glutamic acid or aspartic acid;

X₅ is glutamine or glutamic acid;

X₈ is histidine or phenylalanine;

X₉ and X₁₄ are independently selected from arginine, lysine, ornithine or alanine;

X₁₀ is isoleucine or serine;

X₁₂ is serine or aspartic acid;

X₁₄ is tyrosine, arginine, lysine, ornithine or alanine;

X₁₅ is arginine, lysine, ornithine or leucine;

X₁₇ is glutamic acid or glutamine;

X₁₈ is methionine, asparagine or threonine;

X₁₉ is tyrosine, 4-methoxy-phenylalanine or 4-amino phenylalanine;

X₂₁ is alanine, glycine or asparagine;

X₂₅ is selected from the group consisting of histidine and threonine;

X₂₉ is selected from the group consisting of alanine, glycine and serine;

X₃₀ is selected from the group consisting of histidine, aspartic acid, glutamic acid, homocysteic acid and cysteic acid;

X₃₃ is selected from the group consisting of aspartic acid and glutamic acid;

X₃₄ is selected from the group consisting of alanine and threonine;

X₄₁ is selected from the group consisting of glutamic acid, aspartic acid or asparagine;

X₄₂ is selected from the group consisting of alanine, lysine, ornithine and arginine;

R₆₂ is selected from the group consisting of AYRPSE (SEQ ID NO: 14), FVNQ (SEQ ID NO: 12), PGPE (SEQ ID NO: 11), a tripeptide glycine-proline-glutamic acid, a tripeptide valine-asparagine-glutamine, a dipeptide proline-glutamic acid, a dipeptide asparagine-glutamine, glutamine, glutamic acid and an N-terminal amine; and

R₅₃ is COOH or CONH₂, further wherein the amino acid at the designation X₄₅ is directly bound to the linking moiety, LM (i.e., the designation IB-LM-IA as used herein is intended to represent that the B chain carboxyl terminus and the amino terminus of the A chain are directly linked to the linking moiety LM without any further intervening amino acids).

In one embodiment the linking moiety (LM) comprises an amino acid sequence of no more than 17 amino acids in length. In one embodiment the linking moiety comprises the sequence X₅₁X₅₂GSSSX₅₇X₅₈ (SEQ ID NO: 49) or X₅₁X₅₂GSSSX₅₇X₅₈APQT (SEQ ID NO: 50) wherein X₅₁ is selected from the group consisting of glycine, alanine, valine, leucine, isoleucine and proline, X₅₂ is alanine, valine, leucine, isoleucine or proline and X₅₇ or X₅₈ are independently arginine, lysine, cysteine, homocysteine, acetyl-phenylalanine or ornithine, optionally with a hydrophilic moiety linked to the side chain of the amino acid at position 7 or 8 of the linking moiety (i.e., at the X₅₇ or X₅₈ position). Amino acid positions of the linking moiety are designated based on the corresponding position in the native C chain of IGF 1 (SEQ ID NO: 17). In one embodiment LM is GAGSSSRRAPQT (SEQ ID NO: 23) or GAGSSSRR (SEQ ID NO: 22).

In another embodiment the linking moiety comprises a 29 contiguous amino acid sequence, directly linked to the carboxy terminal amino acid of the B chain, wherein said 29 contiguous amino acid sequence has greater than 70%, 80%, 90% sequence identity to SSSSX₅₀APPPSLPSPSRLPGPSDTPILPQX₅₁ (SEQ ID NO: 68), wherein X₅₀ and X₅₁ are independently selected from arginine and lysine. In one embodiment the linking peptide comprises a total of 29 to 158 or 29 to 58 amino acids and comprises the sequence of SEQ ID NO: 68. In another embodiment the linking moiety comprises a 29 contiguous amino acid sequence, directly linked to the carboxy terminal amino acid of the B chain, wherein said 29 contiguous amino acid sequence has greater than 90% sequence identity to SSSSX₅₀APPPSLPSPSRLPGPSDTPILPQX₅₁ (SEQ ID NO: 68), wherein X₅₀ and X₅₁ are independently selected from arginine and lysine. In one embodiment the linking moiety comprises the sequence SSSSRAPPPSLPSPSRLPGPSDTPILPQK (SEQ ID NO: 51) or SSSSKAPPPSLPSPSRLPGPSDTPILPQR (SEQ ID NO: 52) optionally with one or two amino acid substitutions.

In one embodiment the insulin agonist peptide has the general formula IB-LM-IA wherein IB comprises the sequence GPEHLCGAX₃₀LVDALYLVCGDX₄₂GFYFNX₄₈X₄₉ (SEQ ID NO: 163); LM comprises the sequence SSSSRAPPPSLPSPSRLPGPSDTPILPQK (SEQ ID NO: 51), SSSSKAPPPSLPSPSRLPGPSDTPILPQR (SEQ ID NO: 52), GYGSSSRR (SEQ ID NO: 18), GAGSSSRRAPQT (SEQ ID NO: 23) or GAGSSSRR (SEQ ID NO: 22); and IA comprises the sequence GIVDECCX₈X₉SCDLX₁₄X₁₅LX₁₇X₁₈X₁₉CX₂₁-R₅₃ (SEQ ID NO: 35) wherein

X₈ is histidine or phenylalanine;

X₉ is arginine, ornithine or alanine;

X₁₄ and X₁₅ are both arginine;

X₁₇ is glutamic acid;

X₁₉ is tyrosine, 4-methoxy-phenylalanine or 4-amino phenylalanine;

X₂₁ is alanine or asparagine;

X₂₅ is histidine or threonine;

X₃₀ is selected from the group consisting of histidine, aspartic acid, glutamic acid, homocysteic acid and cysteic acid;

X₄₂ is selected from the group consisting of alanine, ornithine and arginine;

R₅₃ is COOH.

### Linking Moieties for Single Chain Insulin Analogs

Peptide Linkers

In accordance with one embodiment the linking moiety is a peptide or peptidomimetic of 6-18, 8-18, 8-17, 8-12, 8-10, 13-17 or 13-15 amino acids (or amino acid analogs or derivatives thereof). In one embodiment the linking moiety is 8 to 17 amino acids in length and comprises the sequence X₅₁X₅₂GSSSRR (SEQ ID NO: 53) wherein X₅₁ is selected from the group consisting of glycine, alanine, valine, leucine, isoleucine, proline and methionine, and X₅₂ is a non-aromatic amino acid, including for example, alanine. In one embodiment the linking moiety is 8 to 17 amino acids in length and comprises a sequence that differs from X₅₁X₅₂GSSSRR (SEQ ID NO: 53) by a single amino acid substitution wherein the amino acid substitution is an amino acid that is pegylated at its side chain, further wherein X₅₁ is selected from the group consisting of glycine, alanine, valine, leucine, isoleucine, proline and methionine, and X₅₂ is a non-aromatic amino acid, including for example, alanine.

In accordance with one embodiment the linking moiety is a derivative of the IGF 1 C chain sequence (GYGSSSRRAPQT; SEQ ID NO: 17). In one embodiment the derivative is a peptide that differs from SEQ ID NO: 17 by a single amino acid substitution of a lysine, cysteine ornithine, homocysteine, or acetyl-phenylalanine residue, and in a further embodiment the lysine, cysteine ornithine, homocysteine, or acetyl-phenylalanine amino acid is pegylated. In one further embodiment the linking moiety is a peptide that differs from SEQ ID NO: 17 by a single lysine substitution. In one specific embodiment the substitution is made at position 8 of SEQ ID NO: 17. Applicants have discovered that use of the IGF 1 C chain sequence and analogs thereof as a linking moiety will generate a single chain insulin polypeptide that has near wild type insulin activity. Furthermore, use of a IGF 1 C chain sequence analog as the linking moiety, wherein position 2 of the IGF 1 C chain sequence is modified, or the carboxy terminal four amino acids are deleted from the IGF 1 C chain sequence, produces a single chain insulin polypeptide that is selective for insulin (i.e., has a higher binding and/or activity at the insulin receptor compared to the IGF-1 receptor). In one embodiment the single chain insulin polypeptide has 5×, 10×, 20×, 30×, 40×, or 50× higher affinity or activity at the insulin receptor relative to the IGF-1 receptor.

In accordance with one embodiment the linking moiety is a derivative of the IGF 1 C chain sequence (GYGSSSRRAPQT; SEQ ID NO: 17) and comprises a non-native sequence that differs from GYGSSSRR (SEQ ID NO: 18) or GAGSSSRRAPQT (SEQ ID NO: 23) by 1 to 3 amino acid substitutions, or 1 to 2 amino acid substitutions. In one embodiment at least one of the amino acid substitutions is a lysine or cysteine substitution, and in one embodiment the amino acid substitutions are conservative amino acid substitutions. In one embodiment the linking moiety is a peptide (or peptidomimetic) of 8 to 17 amino acids comprising a non-native amino acid sequence that differs from GYGSSSRR (SEQ ID NO: 18) or GAGSSSRRAPQT (SEQ ID NO: 23) by 1 amino acid substitution, including for example substitution with a lysine or cysteine. In one embodiment the linking moiety comprises the sequence GYGSSSRR (SEQ ID NO: 18) or GAGSSSRRAPQT (SEQ ID NO: 23). In one embodiment the linking moiety comprises the sequence GAGSSSRX₅₈APQT (SEQ ID NO: 54), GYGSSSX₅₇X₅₈APQT (SEQ ID NO: 69), or an amino acid that differs from SEQ ID NO: 54 by a single amino acid substitution, wherein X₅₇ is arginine and X₅₈ is arginine, ornithine or lysine, and in a further embodiment a polyethylene glycol chain is linked to the side chain of the amino acid at position 8 of said linking moiety. In another embodiment the linking moiety comprises the sequence GX₅₂GSSSRX₅₈APQT (SEQ ID NO: 55), wherein X₅₂ is any non-aromatic amino acid, including for example, alanine, valine, leucine, isoleucine or proline, and X₅₈ represents an amino acid that has a polyethylene chain covalently linked to its side chain. In one embodiment X₅₈ is a pegylated lysine. In another embodiment, the linking moiety is an 8 to 17 amino acid sequence comprising the sequence GX₅₂GSSSRR (SEQ ID NO: 56), wherein X₅₂ is any amino acid other than Tyr, optionally wherein X₅₂ is Ala.

In accordance with one embodiment the linking moiety is an 8 amino acid sequence selected from the group consisting of GYGSSSRR (SEQ ID NO: 18), GAGSSSRR (SEQ ID NO: 22), GAGSSSRRA (SEQ ID NO: 57), GAGSSSRRAP (SEQ ID NO: 58), GAGSSSRRAPQ (SEQ ID NO: 59), GAGSSSRRAPQT (SEQ ID NO: 23), PYGSSSRR (SEQ ID NO: 61), PAGSSSRR (SEQ ID NO: 62), PAGSSSRRA (SEQ ID NO: 63), PAGSSSRRAP (SEQ ID NO: 64), PAGSSSRRAPQ (SEQ ID NO: 65), PAGSSSRRAPQT (SEQ ID NO: 66). In accordance with one embodiment the linking moiety comprises of consists of an amino acid sequence that differs from GYGSSSRR (SEQ ID NO: 18), GAGSSSRR (SEQ ID NO: 22), GAGSSSRRA (SEQ ID NO: 57), GAGSSSRRAP (SEQ ID NO: 58), GAGSSSRRAPQ (SEQ ID NO: 59), GAGSSSRRAPQT (SEQ ID NO: 23), PYGSSSRR (SEQ ID NO: 61), PAGSSSRR (SEQ ID NO: 62), PAGSSSRRA (SEQ ID NO: 63), PAGSSSRRAP (SEQ ID NO: 64), PAGSSSRRAPQ (SEQ ID NO: 65), PAGSSSRRAPQT (SEQ ID NO: 66) by a single amino acid.

In one embodiment a peptide sequence named C-terminal peptide (CTP: SSSSKAPPPSLPSPSRLPGPSDTPILPQR; SEQ ID NO: 52), which is prone to O-linked hyperglycosylation when the protein is expressed in a eukaryotic cellular expression system, can be used as a linker peptide. In one embodiment the linking moiety comprises an analog of (SEQ ID NO: 68), wherein said analog differs from (SEQ ID NO: 68) by 1, 2, 3, 4, 5 or 6 amino acid substitutions. In one embodiment the linking peptide comprises a CTP peptide wherein amino acid substitutions are made at one or more positions selected from positions 1, 2, 3, 4, 10, 13, 15, and 21 of (SEQ ID NO: 68). In another embodiment the linking moiety comprises a 29 contiguous amino acid sequence, directly linked to the carboxy terminal amino acid of the B chain, wherein said 29 contiguous amino acid sequence has greater than 70%, 80%, 90% sequence identity to SSSSX₅₀APPPSLPSPSRLPGPSDTPILPQX₅₁ (SEQ ID NO: 68), wherein X₅₀ and X₅₁ are independently selected from arginine and lysine, with the proviso that the sequence does not comprise a 15 amino acid sequence identical to a 15 amino acid sequence contained within SEQ ID NO 53. In another embodiment the linking moiety comprises a 29 contiguous amino acid sequence, directly linked to the carboxy terminal amino acid of the B chain, wherein said 29 contiguous amino acid sequence is an analog of (SEQ ID NO: 52), wherein said analog differs from (SEQ ID NO: 52) only by 1, 2, 3, 4, 5 or 6 amino acid modifications, and in a further embodiment the amino acid modifications are conservative amino acid substitutions. In another embodiment the linking moiety comprises a 29 contiguous amino acid sequence, directly linked to the carboxy terminal amino acid of the B chain, wherein said 29 contiguous amino acid sequence is an analog of (SEQ ID NO: 52), wherein said analog differs from (SEQ ID NO: 52) only by 1, 2 or 3 amino acid substitutions.

Applicants have also found that multiple copies of the CTP peptide can be used as the linking peptide in single chain analogs and/or linked to the amino terminus of the B chain in single chain or two chain insulin analogs. The multiple copies of the CTP peptide can be identical or can differ in sequence and can be arranged in a head to tail or head to head orientation. In accordance with one embodiment an insulin analog is provided comprising a CTP peptide having the sequence (SSSSX₅₀APPPSLPSPSRLPGPSDTPILPQX₅₁)ₙ(SEQ ID NO: 68), wherein n is an integer selected from the group consisting of 1, 2, 3 and 4 and X₅₀ and X₅₁ are independently selected from arginine and lysine.

In one embodiment the CTP peptide comprises the sequence SSSSX₅₀APPPSLPSPSRLPGPSDTPILPQX₅₁ (SEQ ID NO: 68), wherein X₅₀ and X₅₁ are independently selected from arginine and lysine. In another embodiment the CTP peptide comprises a sequence selected from the group consisting of SSSSRAPPPSLPSPSRLPGPSDTPILPQK (SEQ ID NO: 51), SSSSKAPPPSLPSPSRLPGPSDTPILPQR (SEQ ID NO: 52) or SSSSRAPPPSLPSPSRLPGPSDTPILPQ (SEQ ID NO: 67), and in a further embodiment the CTP peptide comprises the sequence SSSSRAPPPSLPSPSRLPGPSDTPILPQK (SEQ ID NO: 51).

### Pegylation of Insulin Peptides

Applicants have discovered that covalent linkage of a hydrophilic moiety to the insulin single chain analogs disclosed herein provide analogs having slower onset, extended duration and exhibit a basal profile of activity. In one embodiment, the insulin peptides disclosed herein are further modified to comprise a hydrophilic moiety covalently linked to the side chain of an amino acid at a position selected from the group consisting of A9, A14 and A15 of the A chain or at the N-terminal alpha amine of the B chain (e.g. at position B1 for insulin based B chain or position B2 for IGF-1 based B chain) or at the side chain of an amino acid at position B1, B2, B10, B22, B28 or B29 of the B chain or at any position of the linking moiety that links the A chain and B chain. In exemplary embodiments, this hydrophilic moiety is covalently linked to a Lys, Cys, Orn, homocysteine, or acetyl-phenylalanine residue at any of these positions. In one embodiment the hydrophilic moiety is covalently linked to the side chain of an amino acid of the linking moiety.

Exemplary hydrophilic moieties include polyethylene glycol (PEG), for example, of a molecular weight of about 1,000 Daltons to about 40,000 Daltons, or about 20,000 Daltons to about 40,000 Daltons. Additional suitable hydrophilic moieties include, polypropylene glycol, polyoxyethylated polyols (e.g., POG), polyoxyethylated sorbitol, polyoxyethylated glucose, polyoxyethylated glycerol (POG), polyoxyalkylenes, polyethylene glycol propionaldehyde, copolymers of ethylene glycol/propylene glycol, monomethoxy-polyethylene glycol, mono-(C1-C10) alkoxy- or aryloxy-polyethylene glycol, carboxymethylcellulose, polyacetals, polyvinyl alcohol (PVA), polyvinyl pyrrolidone, poly-1, 3-dioxolane, poly-1,3,6-trioxane, ethylene/maleic anhydride copolymer, poly (beta-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, colonic acids or other polysaccharide polymers, Ficoll or dextran and mixtures thereof.

The hydrophilic moiety, e.g., polyethylene glycol chain in accordance with some embodiments has a molecular weight selected from the range of about 500 to about 40,000 Daltons. In one embodiment the hydrophilic moiety, e.g. PEG, has a molecular weight selected from the range of about 500 to about 5,000 Daltons, or about 1,000 to about 5,000 Daltons. In another embodiment the hydrophilic moiety, e.g., PEG, has a molecular weight of about 10,000 to about 20,000 Daltons. In yet other exemplary embodiment the hydrophilic moiety, e.g., PEG, has a molecular weight of about 20,000 to about 40,000 Daltons. In one embodiment the hydrophilic moiety, e.g. PEG, has a molecular weight of about 20,000 Daltons. In one embodiment an insulin peptide is provided wherein one or more amino acids of the analog are pegylated, and the combined molecular weight of the covalently linked PEG chains is about 20,000 Daltons.

In one embodiment dextrans are used as the hydrophilic moiety. Dextrans are polysaccharide polymers of glucose subunits, predominantly linked by al-6 linkages. Dextran is available in many molecular weight ranges, e.g., about 1 kD to about 100 kD, or from about 5, 10, 15 or 20 kD to about 20, 30, 40, 50, 60, 70, 80 or 90 kD.

Linear or branched polymers are contemplated. Resulting preparations of conjugates may be essentially monodisperse or polydisperse, and may have about 0.5, 0.7, 1, 1.2, 1.5 or 2 polymer moieties per peptide.

In one embodiment the hydrophilic moiety is a polyethylene glycol (PEG) chain, optionally linked to the side chain of an amino acid at a position selected from the group consisting of A9, A14 and A15 of the A chain, positions B1, B2, B10, B22, B28 or B29 of the B chain, at the N-terminal alpha amine of the B chain, or at any position of the linking moiety of a single chain insulin analog that links the A chain and B chain, including for example at position C8. In one embodiment the single chain insulin analog comprises a peptide linking moiety of 8 to 12 amino acids, wherein one of the amino acids of the linking moiety has a polyethylene chain covalently bound to its side chain. In one embodiment the single chain insulin analog comprises a peptide linking moiety of 8 to 12 amino acids, wherein an amino acid of the linking moiety is pegylated and one or more amino acid at a position selected from the group consisting of A9, A14 and A15 of the A chain, positions B1, B2, B10, B22, B28 or B29 of the B chain is also pegylated. In one embodiment the total molecular weight of the covalently linked PEG chain(s) is about 20,000 Daltons.

In one embodiment a single chain insulin analog comprises a linking moiety of 8 to 12 amino acids, wherein one of the amino acids of the linking moiety has a 20,000 Dalton polyethylene chain covalently bound to its side chain. In another embodiment an insulin analog comprises a peptide linking moiety of 8 to 12 amino acids, wherein one of the amino acids of the linking moiety has a polyethylene chain covalently bound to its side chain and a second PEG chain is linked to the N-terminal alpha amine of the B chain (e.g. at position B1 for insulin based B chain or position B2 for IGF-1 based B chain) or at the side chain of an amino acid at position B1, B2 and B29 of the B chain. In one embodiment when two PEG chains are linked to the insulin peptide, each PEG chain has a molecular weight of about 10,000 Daltons. In one embodiment when the PEG chain is linked to an 8 to 12 amino acid linking moiety, the PEG chain is linked at position C7 or C8 of the linking moiety and in one embodiment the PEG chain is linked at position C8 of the linking moiety. In one embodiment when two PEG chains are linked to the single chain insulin analog, with one PEG chain linked at position C8 and the second PEG is linked at A9, A14, A15, B1, B2, B10, B22, B28 or B29.

Hydrophilic moieties such as polyethylene glycol can be attached to the insulin/antagonist peptide conjugates of the present disclosure under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the target compound (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane and 5-pyridyl. If attached to the peptide by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

### Acylation

In some embodiments, the GF21 based conjugate is modified to comprise an acyl group. The acyl group can be covalently linked directly to an amino acid of the bioactive component of the conjugate (i.e., the NHR ligand or the insulin component of conjugate), or indirectly to an amino acid of the NHR ligand or insulin peptide via a spacer, wherein the spacer is positioned between the amino acid of the bioactive component of the conjugate and the acyl group. The conjugate may be acylated at the same amino acid position where a hydrophilic moiety is linked, or at a different amino acid position. For example, acylation may occur at any position including any of amino acid of the conjugate, provided that the activity exhibited by the non-acylated conjugate is retained upon acylation.

In one specific aspect of the invention, the insulin analog is modified to comprise an acyl group by direct acylation of an amine, hydroxyl, or thiol of a side chain of an amino acid of the insulin/antagonist peptide conjugate. In some embodiments, the insulin analog is directly acylated through the side chain amine, hydroxyl, or thiol of an amino acid. In some embodiments, acylation is at position B28 or B29 (according to the amino acid numbering of the native insulin A and B chain sequences). In this regard, an insulin analog can be provided that has been modified by one or more amino acid substitutions in the A or B chain sequence, including for example at positions A14, A15, B1, B2, B10, B22, B28 or B29 (according to the amino acid numbering of the native insulin A and B chain sequences) or at any position of the linking moiety with an amino acid comprising a side chain amine, hydroxyl, or thiol. In some specific embodiments of the invention, the direct acylation of the insulin peptide occurs through the side chain amine, hydroxyl, or thiol of the amino acid at position B28 or B29 (according to the amino acid numbering of the native insulin A and B chain sequences).

In accordance with one embodiment, the acylated insulin analogs comprise a spacer between the peptide and the acyl group. In some embodiments, the insulin/antagonist peptide conjugate is covalently bound to the spacer, which is covalently bound to the acyl group. In some exemplary embodiments, the insulin peptide is modified to comprise an acyl group by acylation of an amine, hydroxyl, or thiol of a spacer, which spacer is attached to a side chain of an amino acid at position B28 or B29 (according to the amino acid numbering of the A or B chain of native insulin), or at any position of the spacer moiety. The amino acid of the insulin/antagonist peptide conjugate to which the spacer is attached can be any amino acid comprising a moiety which permits linkage to the spacer. For example, an amino acid comprising a side chain —NH₂, —OH, or —COOH (e.g., Lys, Orn, Ser, Asp, or Glu) is suitable.

In some embodiments, the spacer between the insulin/antagonist peptide conjugate and the acyl group is an amino acid comprising a side chain amine, hydroxyl, or thiol (or a dipeptide or tripeptide comprising an amino acid comprising a side chain amine, hydroxyl, or thiol). In some embodiments, the spacer comprises a hydrophilic bifunctional spacer. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.). In one embodiment, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate.

In some embodiments, the spacer between the insulin/antagonist peptide conjugate and the acyl group is a hydrophobic bifunctional spacer. Hydrophobic bifunctional spacers are known in the art. See, e.g., Bioconjugate Techniques, G. T. Hermanson (Academic Press, San Diego, Calif., 1996), which is incorporated by reference in its entirety. In accordance with certain embodiments the bifunctional spacer can be a synthetic or naturally occurring amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. Each amino acid of the dipeptide or tripeptide spacer attached to the insulin/antagonist peptide conjugate can be independently selected from the group consisting of: naturally-occurring and/or non-naturally occurring amino acids, including, for example, any of the D or L isomers of the naturally-occurring amino acids (Ala, Cys, Asp, Glu, Phe, Gly, His, Ile, Lys, Leu, Met, Asn, Pro, Arg, Ser, Thr, Val, Trp, Tyr), or any D or L isomers of the non-naturally occurring amino acids selected from the group consisting of: β-alanine (β-Ala), N-α-methyl-alanine (Me-Ala), aminobutyric acid (Abu), α-aminobutyric acid (γ-Abu), aminohexanoic acid (ε-Ahx), aminoisobutyric acid (Aib), aminomethylpyrrole carboxylic acid, aminopiperidinecarboxylic acid, aminoserine (Ams), aminotetrahydropyran-4-carboxylic acid, arginine N-methoxy-N-methyl amide, β-aspartic acid (β-Asp), azetidine carboxylic acid, 3-(2-benzothiazolyl)alanine, α-tert-butylglycine, 2-amino-5-ureido-n-valeric acid (citrulline, Cit), 3-Cyclohexylalanine (Cha), acetamidomethyl-cysteine, diaminobutanoic acid (Dab), diaminopropionic acid (Dpr), dihydroxyphenylalanine (DOPA), dimethylthiazolidine (DMTA), γ-Glutamic acid (γ-Glu), homoserine (Hse), hydroxyproline (Hyp), isoleucine N-methoxy-N-methyl amide, methyl-isoleucine (Melle), isonipecotic acid (Isn), methyl-leucine (MeLeu), methyl-lysine, dimethyl-lysine, trimethyl-lysine, methanoproline, methionine-sulfoxide (Met(O)), methionine-sulfone (Met(O2)), norleucine (Nle), methyl-norleucine (Me-Nle), norvaline (Nva), ornithine (Orn), para-aminobenzoic acid (PABA), penicillamine (Pen), methylphenylalanine (MePhe), 4-Chlorophenylalanine (Phe(4-C1)), 4-fluorophenylalanine (Phe(4-F)), 4-nitrophenylalanine (Phe(4-NO2)), 4-cyanophenylalanine ((Phe(4-CN)), phenylglycine (Phg), piperidinylalanine, piperidinylglycine, 3,4-dehydroproline, pyrrolidinylalanine, sarcosine (Sar), selenocysteine (Sec), U-Benzyl-phosphoserine, 4-amino-3-hydroxy-6-methylheptanoic acid (Sta), 4-amino-5-cyclohexyl-3-hydroxypentanoic acid (ACHPA), 4-amino-3-hydroxy-5-phenylpentanoic acid (AHPPA), 1,2,3,4-tetrahydro-isoquinoline-3-carboxylic acid (Tic), tetrahydropyranglycine, thienylalanine (Thi), U-Benzyl-phosphotyrosine, O-Phosphotyrosine, methoxytyrosine, ethoxytyrosine, O-(bis-dimethylamino-phosphono)-tyrosine, tyrosine sulfate tetrabutylamine, methyl-valine (MeVal), 1-amino-1-cyclohexane carboxylic acid (Acx), aminovaleric acid, beta-cyclopropyl-alanine (Cpa), propargylglycine (Prg), allylglycine (Alg), 2-amino-2-cyclohexyl-propanoic acid (2-Cha), tertbutylglycine (Tbg), vinylglycine (Vg), 1-amino-1-cyclopropane carboxylic acid (Acp), 1-amino-1-cyclopentane carboxylic acid (Acpe), alkylated 3-mercaptopropionic acid, 1-amino-1-cyclobutane carboxylic acid (Acb). In some embodiments the dipeptide spacer is selected from the group consisting of: Ala-Ala, β-Ala-β-Ala, Leu-Leu, Pro-Pro, γ-aminobutyric acid-γ-aminobutyric acid, and γ-Glu-γ-Glu.

The insulin/antagonist peptide conjugate can be modified to comprise an acyl group by acylation of a long chain alkane of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments, the long chain alkane comprises a C₈ to C₂₀ alkane, e.g., a C₁₄ alkane, C₁₆ alkane, or a C₁₈ alkane.

In some embodiments, an amine, hydroxyl, or thiol group of the insulin/antagonist peptide conjugate is acylated with a cholesterol acid. In a specific embodiment, the peptide is linked to the cholesterol acid through an alkylated des-amino Cys spacer, i.e., an alkylated 3-mercaptopropionic acid spacer. Suitable methods of peptide acylation via amines, hydroxyls, and thiols are known in the art. See, for example, Miller, Biochem Biophys Res Commun 218: 377-382 (1996); Shimohigashi and Stammer, Int J Pept Protein Res 19: 54-62 (1982); and Previero et al., Biochim Biophys Acta 263: 7-13 (1972) (for methods of acylating through a hydroxyl); and San and Silvius, J Pept Res 66: 169-180 (2005) (for methods of acylating through a thiol); Bioconjugate Chem. “Chemical Modifications of Proteins: History and Applications” pages 1, 2-12 (1990); Hashimoto et al., Pharmacuetical Res. “Synthesis of Palmitoyl Derivatives of Insulin and their Biological Activity” Vol. 6, No: 2 pp. 171-1⁷⁶ (1989).

The acyl group of the acylated peptide the insulin/antagonist peptide conjugate can be of any size, e.g., any length carbon chain, and can be linear or branched. In some specific embodiments of the invention, the acyl group is a C₄ to C₃₀ fatty acid. For example, the acyl group can be any of a C₄ fatty acid, C₆ fatty acid, C₈ fatty acid, C₁₀ fatty acid, C₁₂ fatty acid, C₁₄ fatty acid, C₁₆ fatty acid, C₁₈ fatty acid, C₂₀ fatty acid, C₂₂ fatty acid, C₂₄ fatty acid, C₂₆ fatty acid, C₂₈ fatty acid, or a C₃₀ fatty acid. In some embodiments, the acyl group is a C₈ to C₂₀ fatty acid, e.g., a C₁₄ fatty acid or a C₁₆ fatty acid.

In an alternative embodiment, the acyl group is a bile acid. The bile acid can be any suitable bile acid, including, but not limited to, cholic acid, chenodeoxycholic acid, deoxycholic acid, lithocholic acid, taurocholic acid, glycocholic acid, and cholesterol acid.

### Alkylation

In some embodiments, the insulin/antagonist peptide conjugate is modified to comprise an alkyl group. The alkyl group can be covalently linked directly to an amino acid of the insulin analog, or indirectly to an amino acid of the insulin/antagonist peptide conjugate via a spacer, wherein the spacer is positioned between the amino acid of the insulin/antagonist peptide conjugate and the alkyl group. The alkyl group can be attached to the insulin/antagonist peptide conjugate via an ether, thioether, or amino linkage. For example, the insulin/antagonist peptide conjugate may be alkylated at the same amino acid position where a hydrophilic moiety is linked, or at a different amino acid position.

Alkylation can be carried out at any position within the insulin/antagonist peptide conjugate, including for example in the C-terminal region of the B chain or at a position in the linking moiety, provided that insulin activity is retained. In a specific aspect of the invention, the insulin/antagonist peptide conjugate is modified to comprise an alkyl group by direct alkylation of an amine, hydroxyl, or thiol of a side chain of an amino acid of the insulin/antagonist peptide conjugate. In some specific embodiments of the invention, the direct alkylation of the insulin/antagonist peptide conjugate occurs through the side chain amine, hydroxyl, or thiol of the amino acid at position A14, A15, B1 (for insulin based B chains), B2 (for IGF-1 based B chains), B10, B22, B28 or B29 (according to the amino acid numbering of the A and B chain of native insulin).

In some embodiments of the invention, the insulin/antagonist peptide conjugate comprises a spacer between the peptide and the alkyl group. In some embodiments, the insulin/antagonist peptide conjugate is covalently bound to the spacer, which is covalently bound to the alkyl group. In some exemplary embodiments, the insulin/antagonist peptide conjugate is modified to comprise an alkyl group by alkylation of an amine, hydroxyl, or thiol of a spacer, wherein the spacer is attached to a side chain of an amino acid at position A14, A15, B1 (for insulin based B chains), B2 (for IGF-1 based B chains), B10, B22, B28 or B29 (according to the amino acid numbering of the A and B chains of native insulin). The amino acid of the insulin/antagonist peptide conjugate to which the spacer is attached can be any amino acid (e.g., a singly α-substituted amino acid or an α,α-disubstituted amino acid) comprising a moiety which permits linkage to the spacer. An amino acid of the insulin/antagonist peptide conjugate comprising a side chain —NH₂, —OH, or —COOH (e.g., Lys, Orn, Ser, Asp, or Glu) is suitable. In some embodiments, the spacer between the insulin/antagonist peptide conjugate and the alkyl group is an amino acid comprising a side chain amine, hydroxyl, or thiol or a dipeptide or tripeptide comprising an amino acid comprising a side chain amine, hydroxyl, or thiol.

In the instance in which the alpha amine is alkylated, the spacer amino acid can be any amino acid. For example, the spacer amino acid can be a hydrophobic amino acid, e.g., Gly, Ala, Val, Leu, Ile, Trp, Met, Phe, Tyr. Alternatively, the spacer amino acid can be an acidic residue, e.g., Asp and Glu. In exemplary embodiments, the spacer amino acid can be a hydrophobic amino acid, e.g., Gly, Ala, Val, Leu, Ile, Trp, Met, Phe, Tyr, 6-amino hexanoic acid, 5-aminovaleric acid, 7-aminoheptanoic acid, 8-aminooctanoic acid. Alternatively, the spacer amino acid can be an acidic residue, e.g., Asp and Glu, provided that the alkylation occurs on the alpha amine of the acidic residue. In the instance in which the side chain amine of the spacer amino acid is alkylated, the spacer amino acid is an amino acid comprising a side chain amine, e.g., an amino acid of Formula I (e.g., Lys or Orn). In this instance, it is possible for both the alpha amine and the side chain amine of the spacer amino acid to be alkylated, such that the peptide is dialkylated. Embodiments of the invention include such dialkylated molecules.

In some embodiments, the spacer comprises a hydrophilic bifunctional spacer. In a specific embodiment, the spacer comprises an amino poly(alkyloxy)carboxylate. In this regard, the spacer can comprise, for example, NH₂(CH₂CH₂O)ₙ(CH₂)ₘCOOH, wherein m is any integer from 1 to 6 and n is any integer from 2 to 12, such as, e.g., 8-amino-3,6-dioxaoctanoic acid, which is commercially available from Peptides International, Inc. (Louisville, Ky.). In some embodiments, the spacer between the insulin/antagonist peptide conjugate and the alkyl group is a hydrophilic bifunctional spacer. In certain embodiments, the hydrophilic bifunctional spacer comprises two or more reactive groups, e.g., an amine, a hydroxyl, a thiol, and a carboxyl group or any combinations thereof. In certain embodiments, the hydrophilic bifunctional spacer comprises a hydroxyl group and a carboxylate. In other embodiments, the hydrophilic bifunctional spacer comprises an amine group and a carboxylate. In other embodiments, the hydrophilic bifunctional spacer comprises a thiol group and a carboxylate.

The spacer (e.g., amino acid, dipeptide, tripeptide, hydrophilic bifunctional spacer, or hydrophobic bifunctional spacer) is 3 to 10 atoms (e.g., 6 to 10 atoms, (e.g., 6, 7, 8, 9, or 10 atoms)) in length. In more specific embodiments, the spacer is about 3 to 10 atoms (e.g., 6 to 10 atoms) in length and the alkyl is a C₁₂ to C₁₈ alkyl group, e.g., C₁₄ alkyl group, C₁₆ alkyl group, such that the total length of the spacer and alkyl group is 14 to 28 atoms, e.g., about 14, 15, 16, 17, 18, 19, 20, 21, 22, 23, 24, 25, 26, 27, or 28 atoms. In some embodiments the length of the spacer and alkyl is 17 to 28 (e.g., 19 to 26, 19 to 21) atoms.

In accordance with one embodiment the bifunctional spacer is a synthetic or non-naturally occurring amino acid comprising an amino acid backbone that is 3 to 10 atoms in length (e.g., 6-amino hexanoic acid, 5-aminovaleric acid, 7-aminoheptanoic acid, and 8-aminooctanoic acid). Alternatively, the spacer can be a dipeptide or tripeptide spacer having a peptide backbone that is 3 to 10 atoms (e.g., 6 to 10 atoms) in length. The dipeptide or tripeptide spacer attached to the insulin/antagonist peptide conjugate can be composed of naturally-occurring and/or non-naturally occurring amino acids, including, for example, any of the amino acids taught herein. In some embodiments the spacer comprises an overall negative charge, e.g., comprises one or two negatively charged amino acids. In some embodiments the dipeptide spacer is selected from the group consisting of: Ala-Ala, β-Ala-β-Ala, Leu-Leu, Pro-Pro, γ-aminobutyric acid-γ-aminobutyric acid, and γ-Glu-γ-Glu. In one embodiment the dipeptide spacer is γ-Glu-γ-Glu.

Suitable methods of peptide alkylation via amines, hydroxyls, and thiols are known in the art. For example, a Williamson ether synthesis can be used to form an ether linkage between the insulin peptide and the alkyl group. Also, a nucleophilic substitution reaction of the peptide with an alkyl halide can result in any of an ether, thioether, or amino linkage. The alkyl group of the alkylated conjugate can be of any size, e.g., any length carbon chain, and can be linear or branched. In some embodiments of the invention, the alkyl group is a C₄ to C₃₀ alkyl. For example, the alkyl group can be any of a C₄ alkyl, C₆ alkyl, C₈ alkyl, C₁₀ alkyl, C₁₂ alkyl, C₁₄ alkyl, C₁₆ alkyl, C₁₈ alkyl, C₂₀ alkyl, C₂₂ alkyl, C₂₄ alkyl, C₂₆ alkyl, C₂₈ alkyl, or a C₃₀ alkyl. In some embodiments, the alkyl group is a C₈ to C₂₀ alkyl, e.g., a C₁₄ alkyl or a C₁₆ alkyl.

In some specific embodiments, the alkyl group comprises a steroid moiety of a bile acid, e.g., cholic acid, chenodeoxycholic acid, deoxycholic acid, lithocholic acid, taurocholic acid, glycocholic acid, and cholesterol acid.

When a long chain alkane is used to alkylate the conjugate or the spacer, the long chain alkane may be of any size and can comprise any length of carbon chain. The long chain alkane can be linear or branched. In certain aspects, the long chain alkane is a C₄ to C₃₀ alkane. For example, the long chain alkane can be any of a C₄ alkane, C₆ alkane, C₈ alkane, C₁₀ alkane, C₁₂ alkane, C₁₄ alkane, C₁₆ alkane, C₁₈ alkane, C₂₀ alkane, C₂₂ alkane, C₂₄ alkane, C₂₆ alkane, C₂₈ alkane, or a C₃₀ alkane. In some embodiments the long chain alkane comprises a C₈ to C₂₀ alkane, e.g., a C₁₄ alkane, C₁₆ alkane, or a C18 alkane.

Also, in some embodiments alkylation can occur between the insulin analog and a cholesterol moiety. For example, the hydroxyl group of cholesterol can displace a leaving group on the long chain alkane to form a cholesterol-insulin peptide product.

### Self-Cleaving Dipeptide Element

In accordance with one embodiment the insulin peptide of the conjugates disclosed herein are further modified to comprise a self-cleaving dipeptide element. In one embodiment the dipeptide element comprises the structure U-J, wherein U is an amino acid or a hydroxyl acid and J is an N-alkylated amino acid. In one embodiment one or more dipeptide elements are linked to the insulin/antagonist peptide conjugate through an amide bond formed through one or more amino groups selected from the N-terminal amino group of the A or B chain of the insulin component, or the side chain amino group of an amino acid present in the conjugate. In accordance with one embodiment one or more dipeptide elements are linked to the insulin/antagonist peptide conjugate at an amino group selected from the N-terminal amino group of the conjugate, or the side chain amino group of an aromatic amine of a 4-amino-phenylalanine residue present at a position corresponding to position A19, B16 or B25 of native insulin, or a side chain of an amino acid of the linking moiety of a single chain insulin analog.

In one embodiment the dipeptide prodrug element comprises the general structure of Formula X:

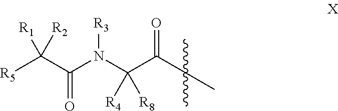

wherein

R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C₁-C₄ alkyl)COOH, (C₁-C₄ alkyl)NH₂, (C₁-C₄ alkyl)NHC(NH₂+)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W)C1-C12 alkyl, wherein W is a heteroatom selected from the group consisting of N, S and O, or R₁ and R₂ together with the atoms to which they are attached form a C₃-C₁₂ cycloalkyl or aryl; or R₄ and R₈ together with the atoms to which they are attached form a C₃-C₆ cycloalkyl;

R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;

R₅ is NHR₆ or OH;

R₆ is H, C₁-C₈ alkyl or R₆ and R₂ together with the atoms to which they are attached form a 4, 5 or 6 member heterocyclic ring; and

R₇ is selected from the group consisting of H and OH. In one embodiment when the prodrug element is linked to the N-terminal amine of the insulin peptide of the insulin/antagonist peptide conjugate and R₄ and R₃ together with the atoms to which they are attached form a 4, 5 or 6 member heterocyclic ring, then at least one of R₁ and R₂ are other than H.

In one embodiment a complex is provided comprising the general structure A-B-(Q-L-Y), wherein Q-L-Y comprises any of the structures as described elsewhere in this disclosure and A-B is a dipeptide that is linked via an amide bond to an amine of the Q-L-Y conjugate. In one embodiment A-B is linked to amine present on the insulin peptide. In one embodiment A-B is linked to the N-terminal alpha amine of the A or B chain of the insulin peptide of the conjugate.

In one embodiment, a complex of the structure A-B-(Q-L-Y) is provided, wherein Q-L-Y comprises any of the structures as described elsewhere in this disclosure and wherein

A is an amino acid or a hydroxy acid;
B is an N-alkylated amino acid linked to Q through an amide bond between a carboxyl moiety of
B and an amine of Q; and
A-B comprises the structure:

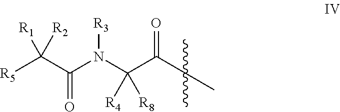

wherein

(a) R¹, R², R⁴ and R⁸ are independently selected from the group consisting of H, C1-C18 alkyl, C2-C18 alkenyl, (C1-C18 alkyl)OH, (C1-C18 alkyl)SH, (C2-C3 alkyl)SCH₃, (C1-C4 alkyl)CONH₂, (C1-C4 alkyl)COOH, (C1-C4 alkyl)NH₂, (C1-C4 alkyl)NHC(NH2⁺)NH₂, (C0-C4 alkyl)(C3-C6 cycloalkyl), (C0-C4 alkyl)(C2-C5 heterocyclic), (C0-C4 alkyl)(C6-C10 aryl)R⁷, (C1-C4 alkyl)(C3-C9 heteroaryl), and C1-C12 alkyl(W1)C1-C12 alkyl, wherein W1 is a heteroatom selected from the group consisting of N, S and O, or
(ii) R¹ and R² together with the atoms to which they are attached form a C3-C12 cycloalkyl or aryl; or
(iii) R⁴ and R⁸ together with the atoms to which they are attached form a C3-C6 cycloalkyl;

(b) R³ is selected from the group consisting of C1-C18 alkyl, (C1-C18 alkyl)OH, (C1-C18 alkyl)NH₂, (C1-C18 alkyl)SH, (C0-C4 alkyl)(C3-C6)cycloalkyl, (C0-C4 alkyl)(C2-C5 heterocyclic), (C0-C4 alkyl)(C6-C10 aryl)R⁷, and (C1-C4 alkyl)(C3-C9 heteroaryl) or R⁴ and R³ together with the atoms to which they are attached form a 4, 5 or 6 member heterocyclic ring;

(c) R⁵ is NHR⁶ or OH;

(d) R⁶ is H, C₁-C₈ alkyl; and

(e) R⁷ is selected from the group consisting of H and OH wherein the chemical cleavage half-life (t_{1/2}) of A-B from Q or Y is at least about 1 hour to about 1 week in PBS under physiological conditions.

In a further embodiment, A-B comprises the structure:

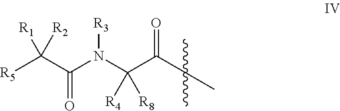

wherein

R₁ and R₈ are independently H or C₁-C₈ alkyl;

R₂ and R₄ are independently selected from the group consisting of H, C₁-C₈ alkyl, (C₁-C₄ alkyl)OH, (C₁-C₄ alkyl)SH, (C₂-C₃ alkyl)SCH₃, (C₁-C₄ alkyl)CONH₂, (C₁-C₄ alkyl)COOH, (C₁-C₄ alkyl)NH₂, and (C₁-C₄ alkyl)(C₆ aryl)R₇;

R₃ is C₁-C₆ alkyl;

R₅ is NH₂; and

R₇ is selected from the group consisting of hydrogen, and OH.

In a further embodiment, A-B comprises the structure:

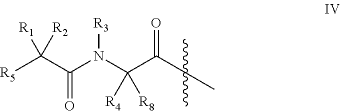

wherein

R₁ is H;

R₂ is H, C₁-C₄ alkyl, (CH₂ alkyl)OH, (C₁-C₄ alkyl)NH₂, or (CH₂)(C₆ aryl)R₇;

R₃ is C₁-C₆ alkyl;

R₄ is H, C₁-C₄ alkyl, or (CH₂)(C₆ aryl)R₇;

R₅ is NH₂;

R₈ is hydrogen; and

R₇ is H or OH.

In a further embodiment, A-B comprises the structure:

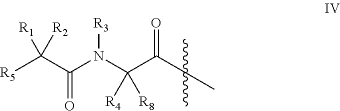

wherein

R₁ is H or C₁-C₄ alkyl;

R₂ is H, C₁-C₄ alkyl, or (C₁-C₄ alkyl)NH₂;

R₃ is C₁-C₆ alkyl;

R₄ is H, or C₁-C₄ alkyl;

R₅ is NH₂; and

R₈ is hydrogen.

Pharmaceutical compositions comprising the insulin/antagonist peptide conjugates disclosed herein can be formulated and administered to patients using standard pharmaceutically acceptable carriers and routes of administration known to those skilled in the art. Accordingly, the present disclosure also encompasses pharmaceutical compositions comprising one or more of the insulin/antagonist peptide conjugates disclosed herein or a pharmaceutically acceptable salt thereof, in combination with a pharmaceutically acceptable carrier. In one embodiment the pharmaceutical composition comprises a 1 mg/ml concentration of the insulin/antagonist peptide conjugate at a pH of about 4.0 to about 7.0 in a phosphate buffer system. The pharmaceutical compositions may comprise the insulin/antagonist peptide conjugate as the sole pharmaceutically active component, or the insulin/antagonist peptide conjugate can be combined with one or more additional active agents.

All therapeutic methods, pharmaceutical compositions, kits and other similar embodiments described herein contemplate that insulin/antagonist peptide conjugates include all pharmaceutically acceptable salts thereof.

In one embodiment the kit is provided with a device for administering the insulin/antagonist peptide conjugate to a patient. The kit may further include a variety of containers, e.g., vials, tubes, bottles, and the like. Preferably, the kits will also include instructions for use. In accordance with one embodiment the device of the kit is an aerosol dispensing device, wherein the composition is prepackaged within the aerosol device. In another embodiment the kit comprises a syringe and a needle, and in one embodiment the conjugate composition is prepackaged within the syringe.

The compounds of this invention may be prepared by standard synthetic methods, recombinant DNA techniques, or any other methods of preparing peptides and fusion proteins. Although certain non-natural amino acids cannot be expressed by standard recombinant DNA techniques, techniques for their preparation are known in the art. Compounds of this invention that encompass non-peptide portions may be synthesized by standard organic chemistry reactions, in addition to standard peptide chemistry reactions when applicable.

### Example 1

Synthesis of Insulin A & B Chains Insulin A & B chains were synthesized on 4-methylbenzhyryl amine (MBHA) resin or 4-Hydroxymethyl-phenylacetamidomethyl (PAM) resin using Boc chemistry. The peptides were cleaved from the resin using HF/p-cresol 95:5 for 1 hour at 0° C. Following HF removal and ether precipitation, peptides were dissolved into 50% aqueous acetic acid and lyophilized. Alternatively, peptides were synthesized using Fmoc chemistry. The peptides were cleaved from the resin using Trifluoroacetic acid (TFA)/Triisopropylsilane (TIS)/H₂O (95:2.5:2.5), for 2 hour at room temperature. The peptide was precipitated through the addition of an excessive amount of diethyl ether and the pellet solubilized in aqueous acidic buffer. The quality of peptides were monitored by RP-HPLC and confirmed by Mass Spectrometry (ESI or MALDI).

Insulin A chains were synthesized with a single free cysteine at amino acid 7 and all other cysteines protected as acetamidomethyl A-(SH)⁷(Acm)^{6,11,20}. Insulin B chains were synthesized with a single free cysteine at position 7 and the other cysteine protected as acetamidomethyl B—(SH)⁷(Acm)¹⁹. The crude peptides were purified by conventional RP-HPLC.

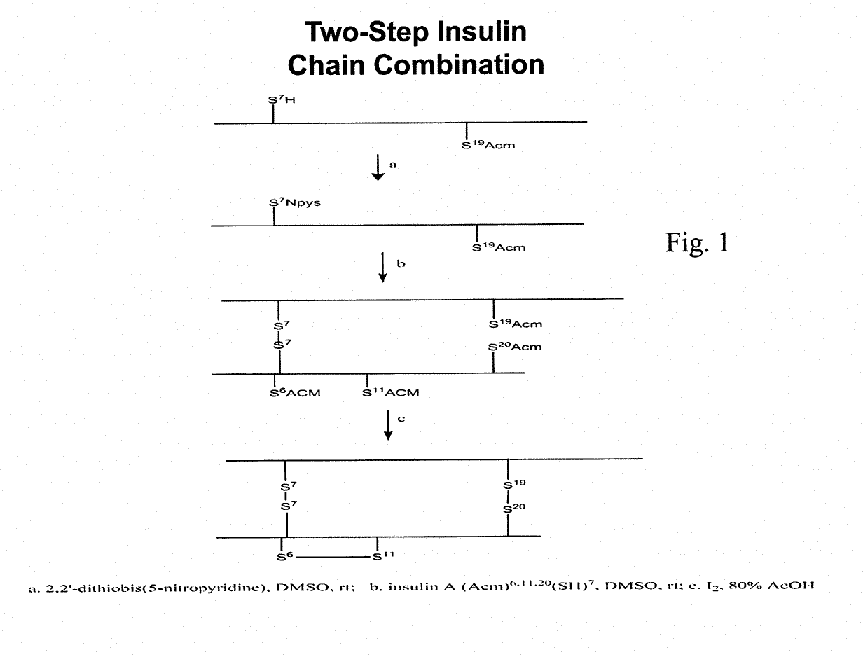

The synthesized A and B chains were linked to one another through their native disulfide bond linkage in accordance with the general procedure outlined in FIG. 1. The respective B chain was activated to the Cys⁷-Npys analog through dissolution in DMF or DMSO and reacted with 2,2′-Dithiobis (5-nitropyridine) (Npys) at a 1:1 molar ratio, at room temperature. The activation was monitored by RP-HPLC and the product was confirmed by ESI-MS.

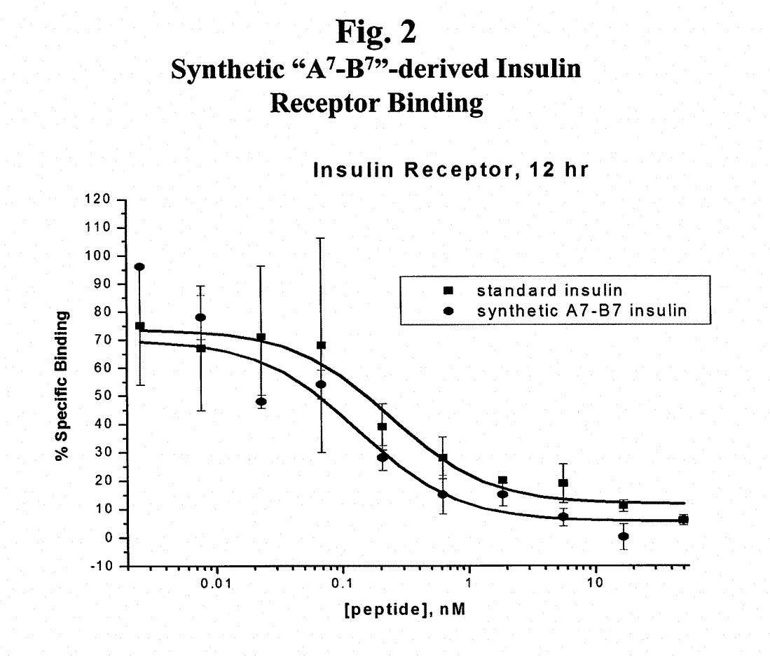

The first B7-A7 disulfide bond was formed by dissolution of the respective A-(SH)⁷(Acm)^{6,11,20} and B-(Npys)⁷(Acm)¹⁹ at 1:1 molar ratio to a total peptide concentration of 10 mg/ml. When the chain combination reaction was complete the mixture was diluted to a concentration of 50% aqueous acetic acid. The last two disulfide bonds were formed simultaneously through the addition of iodine. A 40 fold molar excess of iodine was added to the solution and the mixture was stirred at room temperature for an additional hour. The reaction was terminated by the addition of an aqueous ascorbic acid solution. The mixture was purified by RP-HPLC and the final compound was confirmed by MALDI-MS. As shown in FIG. 2 and the data in Table 1, the synthetic insulin prepared in accordance with this procedure compares well with purified insulin for insulin receptor binding.

Insulin peptides comprising a modified amino acid (such as 4-amino phenylalanine at position A19) can also be synthesized in vivo using a system that allows for incorporation of non-coded amino acids into proteins, including for example, the system taught in U.S. Pat. Nos. 7,045,337 and 7,083,970.

TABLE 1
Activity of synthesized insulin relative to native insulin

| | Insulin Standard | | A7-B7 Insulin | |
|---|---|---|---|---|
| | AVER. | STDEV | AVER. | STDEV |
| IC₅₀(nM) | 0.24 | 0.07 | 0.13 | 0.08 |
| % of Insulin Activity | 100 | | 176.9 | |

### Example 2

### Pegylation of Amine Groups (N-Terminus and Lysine) by Reductive Alkylation

a. Synthesis

Insulin (or an insulin analog), mPEG20k-Aldyhyde, and NaBH₃CN, in a molar ratio of 1:2:30, were dissolved in acetic acid buffer at a pH of 4.1-4.4. The reaction solution was composed of 0.1 N NaCl, 0.2 N acetic acid and 0.1 N Na₂CO₃. The insulin peptide concentration was approximately 0.5 mg/ml. The reaction occurs over six hours at room temperature. The degree of reaction was monitored by RP-HPLC and the yield of the reaction was approximately 50%.

b. Purification

The reaction mixture was diluted 2-5 fold with 0.1% TFA and applied to a preparative RP-HPLC column. HPLC condition: C4 column; flow rate 10 ml/min; A buffer 10% ACN and 0.1% TFA in water; B buffer 0.1% TFA in ACN; A linear gradient B % from 0-40% (0-80 min); PEG-insulin or analogues was eluted at approximately 35% buffer B. The desired compounds were verified by MALDI-TOF, following chemical modification through sulftolysis or trypsin degradation.

### Pegylation of Amine Groups (N-Terminus and Lysine) by N-Hydroxysuccinimide Acylation.

a. Synthesis

Insulin (or an insulin analog) along with mPEG20k-NHS were dissolved in 0.1 N Bicine buffer (pH 8.0) at a molar ratio of 1:1. The insulin peptide concentration was approximately 0.5 mg/ml. Reaction progress was monitored by HPLC. The yield of the reaction is approximately 90% after 2 hours at room temperature.

b. Purification

The reaction mixture was diluted 2-5 fold and loaded to RP-HPLC.

HPLC condition: C4 column; flow rate 10 ml/min; A buffer 10% ACN and 0.1% TFA in water; B buffer 0.1% TFA in ACN; A linear gradient B % from 0-40% (0-80 min); PEG-insulin or analogues was collected at approximately 35% B. The desired compounds were verified by MALDI-TOF, following chemical modification through sulftolysis or trypsin degradation.

### Reductive Aminated Pegylation of Acetyl Group on the Aromatic Ring of the Phenylalanine

a. Synthesis

Insulin (or an insulin analogue), mPEG20k-Hydrazide, and NaBH₃CN in a molar ratio of 1:2:20 were dissolved in acetic acid buffer (pH of 4.1 to 4.4). The reaction solution was composed of 0.1 N NaCl, 0.2 N acetic acid and 0.1 N Na₂CO₃. Insulin or insulin analogue concentration was approximately 0.5 mg/ml. at room temperature for 24 h. The reaction process was monitored by HPLC. The conversion of the reaction was approximately 50%. (calculated by HPLC)

b. Purification

The reaction mixture was diluted 2-5 fold and loaded to RP-HPLC. HPLC condition: C4 column; flow rate 10 ml/min; A buffer 10% ACN and 0.1% TFA in water; B buffer 0.1% TFA in ACN; A linear gradient B % from 0-40% (0-80 min); PEG-insulin, or the PEG-insulin analogue was collected at approximately 35% B. The desired compounds were verified by MALDI-TOF, following chemical modification through sulftolysis or trypsin degradation.

### Example 3

### Insulin Receptor Binding Assay:

The affinity of each peptide for the insulin or IGF-1 receptor was measured in a competition binding assay utilizing scintillation proximity technology. Serial 3-fold dilutions of the peptides were made in Tris-C1 buffer (0.05 M Tris-HCl, pH 7.5, 0.15 M NaCl, 0.1% w/v bovine serum albumin) and mixed in 96 well plates (Corning Inc., Acton, Mass.) with 0.05 nM (3-[125I]-iodotyrosyl) A TyrA14 insulin or (3-[125I]-iodotyrosyl) IGF-1 (Amersham Biosciences, Piscataway, N.J.). An aliquot of 1-6 micrograms of plasma membrane fragments prepared from cells over-expressing the human insulin or IGF-1 receptors were present in each well and 0.25 mg/well polyethylene imine-treated wheat germ agglutinin type A scintillation proximity assay beads (Amersham Biosciences, Piscataway, N.J.) were added. After five minutes of shaking at 800 rpm the plate was incubated for 12 h at room temperature and radioactivity was measured with MicroBeta1450 liquid scintillation counter (Perkin-Elmer, Wellesley, Mass.). Non-specifically bound (NSB) radioactivity was measured in the wells with a four-fold concentration excess of “cold” native ligand than the highest concentration in test samples. Total bound radioactivity was detected in the wells with no competitor. Percent specific binding was calculated as following: % Specific Binding=(Bound-NSB/Total bound-NSB)×100. IC50 values were determined by using Origin software (OriginLab, Northampton, Mass.).

### Example 4

### Insulin Receptor Phosphorylation Assay:

To measure receptor phosphorylation of insulin or insulin analog, receptor transfected HEK293 cells were plated in 96 well tissue culture plates (Costar #3596, Cambridge, Mass.) and cultured in Dulbecco's modified Eagle medium (DMEM) supplemented with 100 IU/ml penicillin, 100 μg/ml streptomycin, 10 mM HEPES and 0.25% bovine growth serum (HyClone SH30541, Logan, Utah) for 16-20 hrs at 37° C., 5% CO₂ and 90% humidity. Serial dilutions of insulin or insulin analogs were prepared in DMEM supplemented with 0.5% bovine serum albumin (Roche Applied Science #100350, Indianapolis, Ind.) and added to the wells with adhered cells. After 15 min incubation at 37° C. in humidified atmosphere with 5% CO₂ the cells were fixed with 5% paraformaldehyde for 20 min at room temperature, washed twice with phosphate buffered saline pH 7.4 and blocked with 2% bovine serum albumin in PBS for 1 hr. The plate was then washed three times and filled with horseradish peroxidase-conjugated antibody against phosphotyrosine (Upstate biotechnology #16-105, Temecula, Calif.) reconstituted in PBS with 2% bovine serum albumin per manufacturer's recommendation. After 3 hrs incubation at room temperature the plate was washed 4 times and 0.1 ml of TMB single solution substrate (Invitrogen, #00-2023, Carlbad, Calif.) was added to each well. Color development was stopped 5 min later by adding 0.05 ml 1 N HCl. Absorbance at 450 nm was measured on Titertek Multiscan MCC340 (ThermoFisher, Pittsburgh, Pa.). Absorbance vs. peptide concentration dose response curves were plotted and EC₅₀ values were determined by using Origin software (OriginLab, Northampton, Mass.).

### Example 5

### Determination of Rate of Model Dipeptide Cleavage (in PBS)

A specific hexapeptide (HSRGTF-NH₂; SEQ ID NO: 72) was used as a model peptide upon which the rate of cleavage of dipeptide N-terminal extensions could be studied. The dipeptide-extended model peptides were prepared Boc-protected sarcosine and lysine were successively added to the model peptide-bound resin to produce peptide A (Lys-Sar-HSRGTF-NH₂; SEQ ID NO: 73). Peptide A was cleaved by HF and purified by preparative HPLC.

Preparative purification using HPLC: Purification was performed using HPLC analysis on a silica based 1×25 cm Vydac C18 (5μ particle size, 300 A⁰ pore size) column. The instruments used were: Waters Associates model 600 pump, Injector model 717, and UV detector model 486. A wavelength of 230 nm was used for all samples. Solvent A contained 10% CH₃CN/0.1% TFA in distilled water, and solvent B contained 0.1% TFA in CH₃CN. A linear gradient was employed (0 to 100% B in 2 hours). The flow rate was 10 ml/min and the fraction size was 4 ml. From ˜150 mgs of crude peptide, 30 mgs of the pure peptide was obtained.

Peptide A was dissolved at a concentration of 1 mg/ml in PBS buffer. The solution was incubated at 37° C. Samples were collected for analysis at 5 h, 8 h, 24 h, 31 h, and 47 h. The dipeptide cleavage was quenched by lowering the pH with an equal volume of 0. I1% TFA. The rate of cleavage was qualitatively monitored by LC-MS and quantitatively studied by HPLC. The retention time and relative peak area for the prodrug and the parent model peptide were quantified using Peak Simple Chromatography software.

### Analysis Using Mass Spectrometry

The mass spectra were obtained using a Sciex API-III electrospray quadrapole mass spectrometer with a standard ESI ion source. Ionization conditions that were used are as follows: ESI in the positive-ion mode; ion spray voltage, 3.9 kV; orifice potential, 60 V. The nebulizing and curtain gas used was nitrogen flow rate of 0.9 L/min. Mass spectra were recorded from 600-1800 Thompsons at 0.5 Th per step and 2 msec dwell time. The sample (about 1 mg/mL) was dissolved in 50% aqueous acetonitrile with 1% acetic acid and introduced by an external syringe pump at the rate of 5 μL/min. Peptides solubilized in PBS were desalted using a ZipTip solid phase extraction tip containing 0.6 μL C4 resin, according to instructions provided by the manufacturer (Millipore Corporation, Billerica, Mass.) prior to analysis.

### Analysis Using HPLC

The HPLC analyses were performed using a Beckman System Gold Chromatography system equipped with a UV detector at 214 nm and a 150 mm×4.6 mm C8 Vydac column. The flow rate was 1 ml/min. Solvent A contained 0.1% TFA in distilled water, and solvent B contained 0.1% TFA in 90% CH₃CN. A linear gradient was employed (0% to 30% B in 10 minutes). The data were collected and analyzed using Peak Simple Chromatography software.

The rate of cleavage was determined for the respective propeptides. The concentrations of the propeptides and the model parent peptide were determined by their respective peak areas. The first order dissociation rate constants of the prodrugs were determined by plotting the logarithm of the concentration of the prodrug at various time intervals. The slope of this plot provides the rate constant ‘k’. The half lives for cleavage of the various prodrugs were calculated by using the formula t_{1/2}=0.693/k. The half life of the Lys-Sar extension to this model peptide HSRGTF-NH₂ (SEQ ID NO: 72) was determined to be 14.0 h.

### Example 6

Rate of Dipeptide Cleavage Half Time in Plasma Using an all d-Isoform Model Peptide

An additional model hexapeptide (dHdTdRGdTdF-NH₂ SEQ ID NO: 73) was used to determine the rate of dipeptide cleavage in plasma. The d-isomer of each amino acid was used to prevent enzymatic cleavage of the model peptide, with the exception of the prodrug extension. This model d-isomer hexapeptide was synthesized in an analogous fashion to the 1-isomer. The sarcosine and lysine were successively added to the N-terminus as reported previously for peptide A to prepare peptide B (dLys-dSar-dHdTdRGdTdF-NH₂ SEQ ID NO: 74) The rate of cleavage was determined for the respective propeptides. The concentrations of the propeptides and the model parent peptide were determined by their respective peak areas.

The first order dissociation rate constants of the prodrugs were determined by plotting the logarithm of the concentration of the prodrug at various time intervals. The slope of this plot provides the rate constant ‘k’. The half life of the Lys-Sar extension to this model peptide dHdTdRGdTdF-NH₂ (SEQ ID NO: 73) was determined to be 18.6 h.

### Example 7

The rate of cleavage for additional dipeptides linked to the model hexapeptide (HSRGTF-NH₂; SEQ ID NO: 72) were determined using the procedures described in Example 5. The results generated in these experiments are presented in Tables 2 and 3.

TABLE 2
Cleavage of the Dipeptide U-B that are linked to the side chain of an
N-terminal para-amino-Phe from the Model Hexapeptide
(HSRGTF-NH₂; SEQ ID NO: 72) in PBS

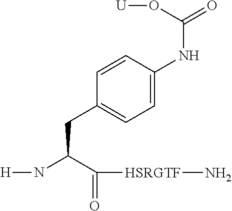

| Compounds | U (amino acid) | O (amino acid) | t_{1/2} | |
|---|---|---|---|---|
| 1 | F | P | 58 | h |
| 2 | Hydroxyl-F | P | 327 | h |
| 3 | d-F | P | 20 | h |
| 4 | d-F | d-P | 39 | h |
| 5 | G | P | 72 | h |
| 6 | Hydroxyl-G | P | 603 | h |
| 7 | L | P | 62 | h |
| 8 | tert-L | P | 200 | h |
| 9 | S | P | 34 | h |
| 10 | P | P | 97 | h |
| 11 | K | P | 33 | h |
| 12 | dK | P | 11 | h |
| 13 | E | P | 85 | h |
| 14 | Sar | P | ≈1000 | h |
| 15 | Aib | P | 69 | min |
| 16 | Hydroxyl-Aib | P | 33 | h |
| 17 | cyclohexane | P | 6 | min |
| 18 | G | G | No cleavage | |
| 19 | Hydroxyl-G | G | No cleavage | |
| 20 | S | N-Methyl-Gly | 4.3 | h |
| 21 | K | N-Methyl-Gly | 5.2 | h |
| 22 | Aib | N-Methyl-Gly | 7.1 | min |
| 23 | Hydroxyl-Aib | N-Methyl-Gly | 1.0 | h |

TABLE 3
Cleavage of the Dipeptides U-B linked to histidine (or histidine
analog) at position 1 (X) from the Model Hexapeptide
(XSRGTF-NH₂; SEQ ID NO: 75) in PBS
NH₂-U-B-XSRGTF-NH₂ (SEQ ID NO: 75)

| Comd. | U (amino acid) | O (amino acid) | X (amino acid) | t_{1/2} | |
|---|---|---|---|---|---|
| 1 | F | P | H | No cleavage | |
| 2 | Hydroxyl-F | P | H | No cleavage | |
| 3 | G | P | H | No cleavage | |
| 4 | Hydroxyl-G | P | H | No cleavage | |
| 5 | A | P | H | No cleavage | |
| 6 | C | P | H | No cleavage | |
| 7 | S | P | H | No cleavage | |
| 8 | P | P | H | No cleavage | |
| 9 | K | P | H | No cleavage | |
| 10 | E | P | H | No cleavage | |
| 11 | Dehydro V | P | H | No cleavage | |
| 12 | P | d-P | H | No cleavage | |
| 13 | d-P | P | H | No cleavage | |
| 14 | Aib | P | H | 32 | h |
| 15 | Aib | d-P | H | 20 | h |
| 16 | Aib | P | d-H | 16 | h |
| 17 | Cyclohexyl- | P | H | 5 | h |
| 18 | Cyclopropyl- | P | H | 10 | h |
| 19 | N—Me-Aib | P | H | >500 | h |
| 20 | α,α-diethyl- | P | H | 46 | h |
| | Gly | | | | |
| 21 | Hydroxyl-Aib | P | H | 61 | |
| 22 | Aib | P | A | 58 | |
| 23 | Aib | P | N-Methyl-His | 30 | h |
| 24 | Aib | N-Methyl-Gly | H | 49 | min |
| 25 | Aib | N-Hexyl-Gly | H | 10 | min |
| 26 | Aib | Azetidine-2- | H | >500 | h |
| | | carboxylic acid | | | |
| 27 | G | N-Methyl-Gly | H | 104 | h |
| 28 | Hydroxyl-G | N-Methyl-Gly | H | 149 | h |
| 29 | G | N-Hexyl-Gly | H | 70 | h |
| 30 | dK | N-Methyl-Gly | H | 27 | h |
| 31 | dK | N-Methyl-Ala | H | 14 | h |
| 32 | dK | N-Methyl-Phe | H | 57 | h |
| 33 | K | N-Methyl-Gly | H | 14 | h |
| 34 | F | N-Methyl-Gly | H | 29 | h |
| 35 | S | N-Methyl-Gly | H | 17 | h |
| 36 | P | N-Methyl-Gly | H | 181 | h |

### Example 8

Insulin Like Growth Factor (IGF) Analog IGF1 (Y^{B16}L^{B17})

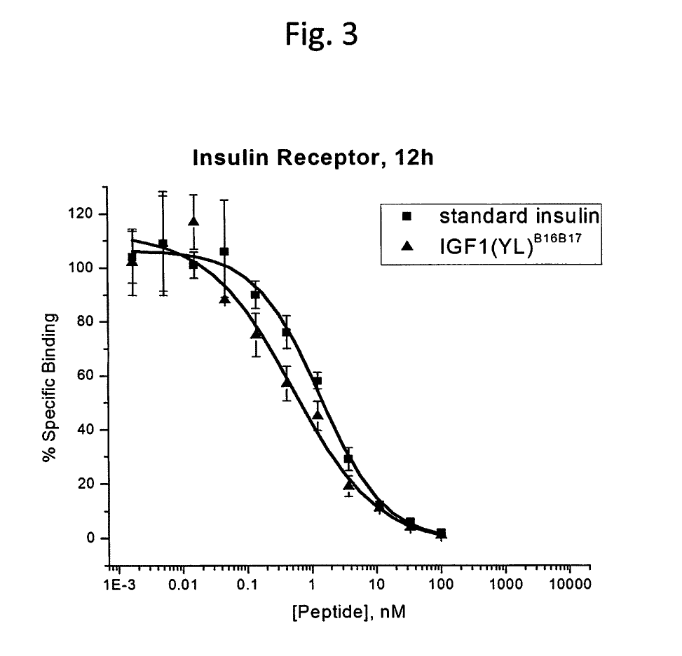

Applicants have discovered an IGF analog that demonstrates similar activity at the insulin receptor as native insulin. More particularly, the IGF analog (IGF1 (Y^{B16}L^{B17}) comprises the native IGFI A chain (SEQ ID NO: 5) and the modified IGFI B chain (SEQ ID NO: 6), wherein the native glutamine and phenylalanine at positions 15 and 16 of the native IGF B-chain (SEQ ID NO: 3) have been replaced with tyrosine and leucine residues, respectively. As shown in FIG. 3 and Table 4 below the binding activities of IGF1 (Y^{B16}L^{B17}) and native insulin demonstrate that each are highly potent agonists of the insulin receptor.

TABLE 4

| | Insulin Standard | | IGF1(Y^{B16}L^{B17}) | |
|---|---|---|---|---|
| | AVER. | STDEV | AVER. | STDEV |
| IC₅₀(nM) | 1.32 | 0.19 | 0.51 | 0.18 |
| % of Insulin Activity | 100 | | 262 | |

### Example 9

### Acylated Insulin Analogs

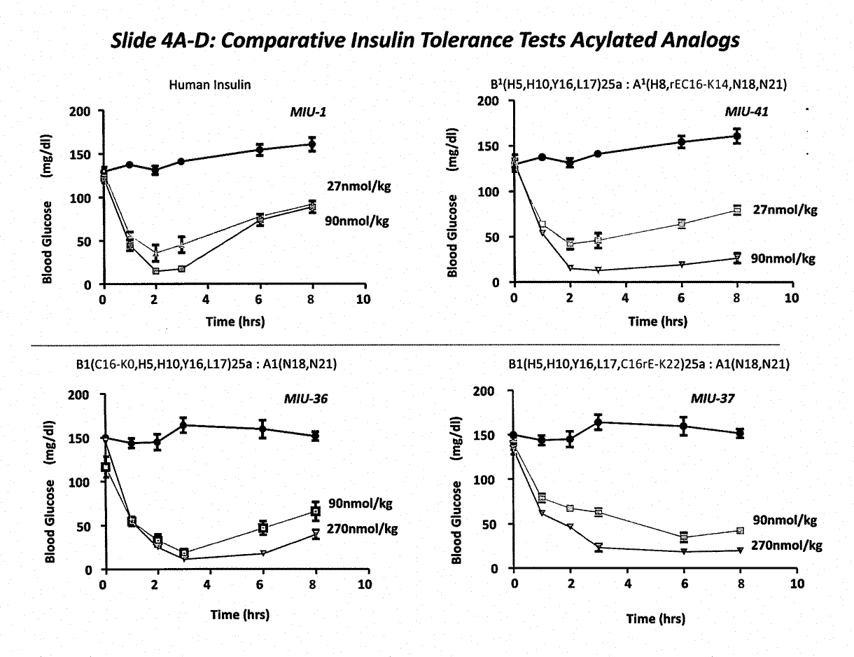

Comparative insulin tolerance tests were conducted on mice comparing the ability of human insulin relative to three different acylated insulin analogs to reduce and sustain low blood glucose concentration. The compounds were tested at two different concentrations (27 nmol/kg and 90 nmol/kg). The acylated insulins included MIU-41 (a two chain insulin analog having a C16 acylation via a gamma glutamic acid linker attached to a lysine residue located at position A14), MIU-36 (a two chain insulin analog having a C16 acylation linked to the N-terminus of the B chain) and MIU-37 (a two chain insulin analog having a C16 acylation via a gamma glutamic acid linker attached to a lysine residue located at position B22). All three acylated insulin analogs provided a more basal and sustained lowered glucose levels relative to native insulin, even after 8 hours (See FIGS. 4A-4D).

### Example 10

Insulin/antagonist peptide conjugates

### Methods

Insulin/antagonist peptide conjugates were created by ligating antagonist peptides to a modified insulin through a disulfide bond. Native insulin contains three primary amines, one at the N-terminus of the A chain, one at the N-terminus of the B chain, and a third present in the side chain of the only native lysine residue, the B29 position. The difference in reactivity between the N-terminal amines and the lysine side chain amine allows for the specific functionalization of the lysine residue.

Native insulin (Eli Lilly and Co), was reacted with a specific NHS ester. The ester itself was created by reaction of S-trityl-β-mercaptopropionic acid (National Biochemicals Corporation) with N-hydroxy succinimide (Chem Impex International) and diisopropylcarbodiimide (Aldrich) in a 1:1:0.9 ratio in anhydrous DMF (Aldrich). After centrifuging to precipitate unwanted side products, the NHS ester was reacted in a 1:1 molar ratio with native insulin in 25 mM boric acid buffer, 50% acetonitrile, pH 10.2. The modification was confirmed by LC/MS, which shows a single modification by mass. This peptide was purified using reverse phase chromatography with a silica based C8 column. The trityl protecting group was then removed with anhydrous TFA. The specificity of the B29 modification was determined by subjecting the modified peptide to a trypsin digest, which removes the last eight residues of the B chain, including the B29 lysine residue. The corresponding decrease in mass demonstrated that the N-terminal amines of the A and B chain remained unmodified.

The trityl-protecting group of the modified insulin is subsequently removed with anhydrous TFA and activated with 20 molar equivalents of 2,2′-dithiobis(5-nitropyridine) (DTNP), to yield an activated disulfide. 5% triisopropylsilane was included in this reaction to quench the trityl cations. The peptide was then purified using reverse phase chromatography. This peptide contains an activated disulfide that reacts with any free sulfhydryl group to create a disulfide bond. This enables the formation of a disulfide bond between the activated insulin and any peptide that contains a single free sulfhydryl.

All other peptides described in this Example were synthesized using Fmoc chemistry on a Chemmatrix rink amide resin. Peptides were cleaved from the resin in a cleavage cocktail of 2.5% triisopropylsilane, β-mercaptoethanol, thioanisol, and H2O in TFA. Crude peptides often contained multiple peaks as shown by LC/MS. Stirring the crude peptide in a dilute acid solution, such as 2% acetic acid or 1% acetic acid, 20% acetonitrile overnight resolved the crude peptide into a single peak with the correct charge to mass ratio, as shown by LC/MS.

After Fmoc synthesis and cleavage, peptides were precipitated with ether, dissolved in 20% acetonitrile, 1% acetic acid solution and stirred overnight to remove any remaining protecting groups. Peptides were then lyophilized and subsequently purified using reverse phase chromatography. LC/MS was used to confirm the purity and accuracy of the synthesis.

Ligation reactions occurred in 50 mM sodium phosphate buffer, 20% acetonitrile, pH 6.0-7.0. A peptide containing a single free thiol group and activated insulin were mixed in a 6:1 molar ratio and dissolved in 50 mM sodium phosphate/20% acetonitrile buffer to a concentration of 20 mg/mL. Reaction was monitored by LC/MS. The ligated peptide was subsequently purified using reverse phase FPLC with an Amberchrome XT-20 divinylbenzyl polystyrene column. LC/MS was used to confirm the mass and analyze the purity of the ligated peptides. The ligated peptides were then dissolved in 25 mM ammonium bicarbonate buffer, pH 8, and analyzed for concentration using UV-Vis nanodrop spectroscopy. These peptides in solution were subsequently analyzed in a phosphorylation assay for activity at the insulin receptor A and B isoforms, as described in Example 4. Optical density at 450 nm was graphed as a function of peptide concentration using Origin Pro 9.0 graphing software. Chemical reactions and schematics in this chapter were created with ChemBioDraw Ultra 13.0.

### EXPERIMENTAL

Insulin/antagonist peptide conjugates were prepared by the formation of a disulfide bond between an insulin analog and an antagonist peptide. To do so, we first chemically modified native human insulin by exploiting the unique reactivity of its only lysine residue, which is at position B29. Since the B chain has only 30 residues, this lysine is ideally situated near the C terminus of the B chain and is not involved in receptor binding. We were able to modify this residue by reacting native insulin with an Nhydroxysuccinimide (NHS) ester at an aqueous pH of 10. At this pH, only the ε-amine of the lysine residue reacts with the ester, sparing reactivity at the two unprotected Nterminal amines. The NHS ester contains a trityl-protected thiol at the other end of the reagent, such that native insulin can be modified to form an amide at B29 that extends to include the protected thiol function group. Removal of the trityl-protecting group, followed by activation with 2,2′-dithiobis(5-nitropyridine) (DTNP) yields an insulin analog that will readily react with free thiols in aqueous solution at relatively low pH to form a disulfide bond. Therefore, an array of conjugates could by synthesized by reacting the activated insulin with peptides that contain a single cysteine residue (Table 5). The advantage of this approach is that it minimizes the number of modifications required to covalently bond unique antagonist peptides to insulin. It is also highly specific, and compatible with peptides created by solid phase peptide synthesis. The conjugates created using this approach were tested for the degree of biological action at the insulin receptor isoforms.

Further optimization of the specific sequence of the antagonist peptide followed by analogous conjugation to insulin led to the discovery of a library of conjugates possessing high potency and variable maximal activity.

### Results

Native insulin was modified through the use of a specific NHS ester to create an insulin molecule with one additional thiol functional group at the B29 lysine (FIG. 5). In order to ensure that this modification did not interfere with binding to the insulin receptor, the modified insulin was tested for agonism at both insulin receptors. It was found that the modified insulin was still capable of fully activating the insulin receptor isoforms, although with slightly reduced potency. The modified insulin was chemically activated for thiol-conjugation by removing the trityl protecting group with TFA and reacting the resulting free thiol with DTNP. The activated insulin was purified and subsequently conjugated to peptides containing a free thiol to yield a specific disulfide bond.

The first set of conjugates consisted of the modified insulin and the site 1 binding motif possessed by peptide #4 (Table 5). Insulin conjugated to #Cys4 (#Insulin-Cys-4) displays only a small change in maximal activity and a small decrease in potency when compared to native insulin. However, in contrast the insulin conjugated to #4Cys (#4-Cys-Insulin) does display some degree of antagonism at both receptor isoforms at higher peptide concentrations.

TABLE 5
Names and sequences of peptides containing single
cysteine residues

| Peptide | |
|---|---|
| Reference | |
| Number | Sequence |
| Cys4 | CGSLDESFYDWFERQLG |
| | (SEQ ID NO: 166) |
| 4Cys | GSLDESFYDWFERQLGC |
| | (SEQ ID NO: 167) |
| Cys6 | CSLEEEWAQIQSEVWGRGSPSY |
| | (SEQ ID NO: 168) |
| 6Cys | SLEEEWAQIQSEVWGRGSPSYC |
| | (SEQ ID NO: 169) |
| 6(des1-5)Cys | WAQIQSEVWGRGSPSYC |
| | (SEQ ID NO: 170) |
| 6(A1)Cys | ALEEEWAQIQSEVWGRGPSYC |
| | (SEQ ID NO: 171) |
| 6(A2)Cys | SAEEEWAQIQSEVWGRGSPSYC |
| | (SEQ ID NO: 172) |
| 6(A3)Cys | SLAEEWAQIQSEVWGRGSPSYC |
| | (SEQ ID NO: 173) |
| 6(L2)Cys* | SLEEEWAQIQSEVWGRGSPSYC |
| | (SEQ ID NO: 169) |
| 6(dL2)Cys** | SdLEEEWAQIQSEVWGRGSPSYC |
| | (SEQ ID NO: 174) |
| 6(I2)Cys | SIEEEWAQIQSEVWGRGSPSYC |
| | (SEQ ID NO: 175) |
| 6(V2)Cys | SVEEEWAQIQSEVWGRGSPSYC |
| | (SEQ ID NO: 176) |
| 6(F2)Cys | SFEEEWAQIQSEVWGRGSPSYC |
| | (SEQ ID NO: 177) |
| 6(W2)Cys | SWEEEWAQIQSEVWGRGSPSYC |
| | (SEQ ID NO: 178) |
| 6(Y2)Cys | SYEEEWAQIQSEVWGRGSPSYC |
| | (SEQ ID NO: 179) |
| 6(Q2)Cys | SQEEEWAQIQSEVWGRGSPSYC |
| | (SEQ ID NO: 180) |

*same as 6Cys, renamed to emphasize the identity of the residue at position two.
**dL refers to the d-stereochemistry of the leucine residue at position two. All other amino acids are of 1-stereochemistry

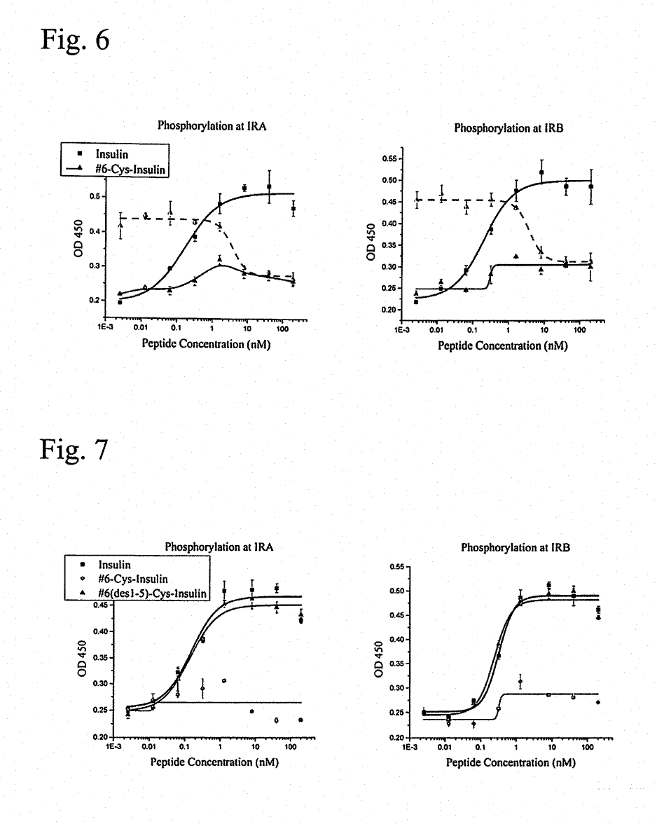

The next set of conjugates prepared and biologically characterized contains the site 2 binding motif possessed by peptide #6 (Table 5). When insulin is conjugated to #6-Cys, the conjugate is completely inactive at both insulin receptor isoforms (FIG. 6). This is not merely the result of a lack of receptor binding, since this peptide, #6-Cys-Insulin, is capable of fully antagonizing exogenously added native insulin (FIG. 6). This was a surprising result, since previous work has shown that single binding site motifs, including #6Cys, are incapable of antagonizing native insulin when applied as a non-covalent addition (in trans). Therefore, the conjugation of this peptide to insulin results in a molecule that has emergent antagonism, relative to what is observed for the individual constituent peptides.

We further investigated the activity of the #6-Cys-Insulin conjugate by creating a truncated version of the #6 motif, #6(des1-5)-Cys, which is devoid of the first five N-terminal amino acids (Table 5). The #6(des1-5)-Cys-Insulin conjugate was tested for activity at the insulin receptor isoforms, and it was found that removal of the N-terminal pentapeptide of the #6 binding motif restored the conjugate to full activity, and full potency (FIG. 7). This astonishing result demonstrates that the antagonistic activity of this conjugate can be completely controlled by the first five residues of the #6 motif.

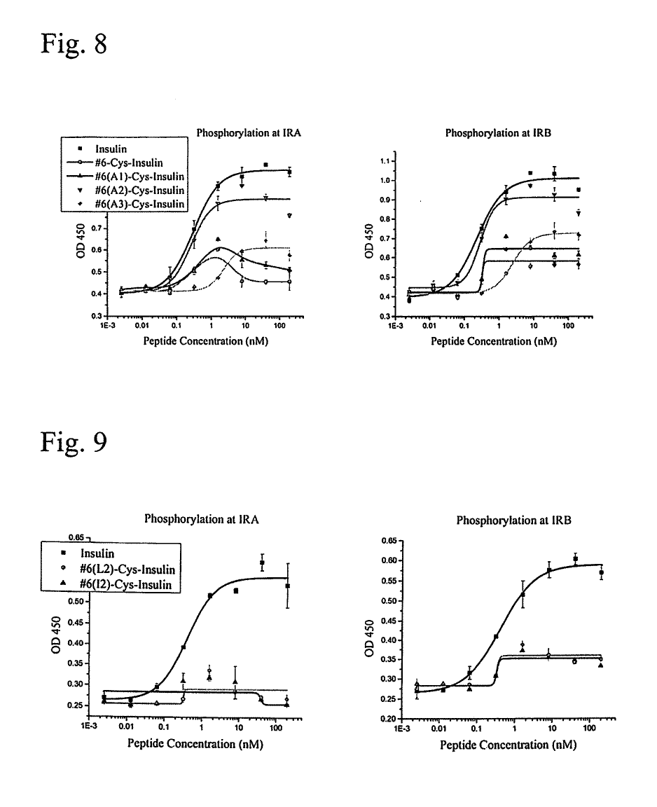

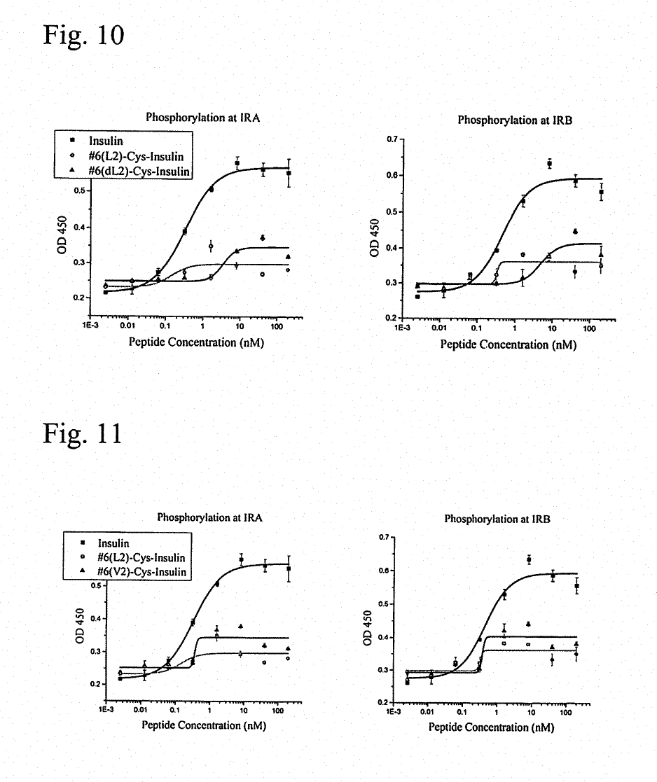

To interrogate this structure-activity relationship further, an alanine scan of the N-terminal residues of the #6 motif was employed to create conjugates with site-specific mutation of the conjugated #6-Cys antagonist peptide. Relative to the N-terminus of the #6 sequence, an alanine in the first position had almost no effect upon bioactivity, relative to the unaltered conjugate (FIG. 8). An alanine in the second position, however, restored almost all of the potency and activity to the conjugate (FIG. 8). An alanine in the third position seemed to shift the potency of the conjugate, but had little effect on maximal activity (FIG. 8). Based on these results, we shifted the attention to the second amino acid in the site 2 binding motif, a leucine.

A series of mutations to the second position of the #6-Cys antagonist were made (Table 5) and these peptides were similarly conjugated via a disulfide to B29 modified insulin. Most of these mutations affected the maximal activity of the conjugate without a large shift in potency. It was found that leucine and isoleucine had very similar, low activities (FIG. 9). However, substituting d-leucine at position two resulted in a low activity conjugate with decreased potency (FIG. 14). This suggests that there is an appropriate size, hydrophobicity and chirality that results in low activity conjugates.

Based upon the observations with leucine, isoleucine, and d-leucine, we decided to further explore hydrophobic residues at position two. Substituting to valine at position two results in a slight increase in activity (FIG. 11). Substituting to phenylalanine at position two results in a further increase in activity, and begins to approach 50% activity at both receptor isoforms (FIG. 12). Substituting to tryptophan at position two results in a conjugate with approximately 60% the activity of native insulin (FIG. 13), and the final substitution to tyrosine, results in a conjugate with approximately 70% the activity of native insulin (FIG. 14). The introduction of a more hydrophilic residue, glutamine, was also investigated at position two (FIG. 15). This mutation resulted in a shift in potency, without a significant decrease in maximal activity. The most satisfying aspect of these modifications is that all of the conjugates with hydrophobic residues at position two maintain a high inherent potency. This represents the discovery of a library of peptides with high inherent potency, but variable maximal activity, as was the primary goal of this research.

### Example 11

### In Vivo Administration of Insulin/Antagonist Peptide Conjugates.

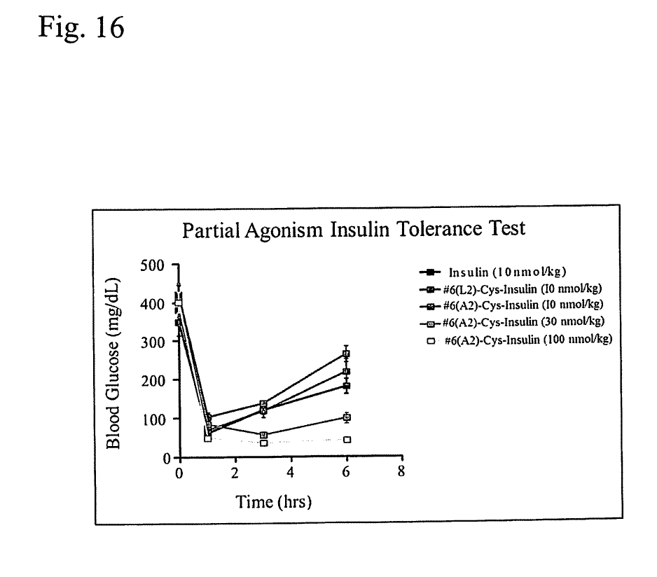

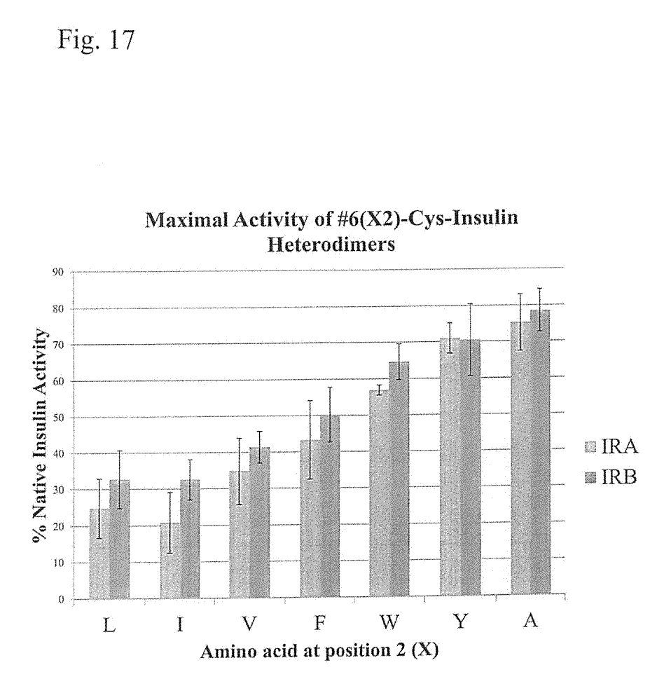

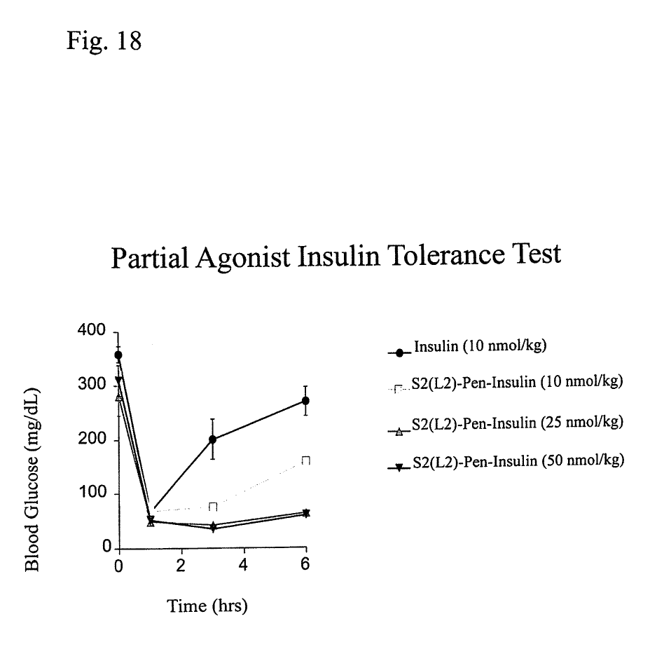

Two insulin/antagonist conjugates, #6(L2)-Cys-Insulin and #6(A2)-Cys-Insulin were administered to mice and their impact on blood glucose levels was determined. The peptides were tested in STZ-mice, due to their high fasting blood glucose levels. The insulin/antagonist peptide conjugates #6(L2)-Cys-Insulin and #6(A2)-Cys-Insulin were chosen for study, since these represent the lowest and highest activity conjugates in our library, and therefore represented the greatest possible dynamic range. Mice were fasted for 2 hours prior to the injection of native human insulin, #6(L2)-Cys-Insulin, or #6(A2)-Cys-Insulin. Insulin and #6(A2)-Cys-Insulin were administered at 10 nmol/kg doses. In addition, #6(L2)-Cys-Insulin was administered at 10, 30, and 100 nmol/kg doses. Each data point represents the average of 8 mice. It was found that the high-activity #6(A2)-Cys-Insulin conjugate behaved identically to an equivalent dose of native insulin (FIG. 16).

However, the low activity conjugate, #6(L2)-Cys-Insulin, also behaved similarly to an equivalent dose of native insulin (FIG. 16). Further increases in dosing led to delayed decreases in blood glucose, to the extent that some of the animals exhibited signs of hypoglycemia (seizures, hypothermia), and had to be rescued with exogenous glucose. These results suggest that diminished activity at the insulin receptor in vitro does not translate to diminished activity in vivo. However, this may be due to peptide instability in this in vivo assay. The in vitro phosphorylation assay does not fully mimic a plasma exposure, which can contain reduction cofactors, and enzymes that can participate in the reduction of disulfide bonds. If the insulin conjugates are not stable in vivo, then the breakage of the disulfide bond would result in a fully potent agonist, and the #6-Cys antagonist, which is incapable of antagonism in vitro, as shown previously in chapter 2.

To address the possibility of disulfide instability, a slightly modified version of the #6(L2)-Cys-Insulin molecule was created, where the cysteine residue has been replaced by penicillamine, which is simply a gem-dimethyl form of cysteine. This new conjugate, #6(L2)-Pen-Insulin, behaves identically to its cysteine counterpart in the in vitro assays. The di-methylation should yield the disulfide bond additional in vivo stability, since it is less susceptible to enzymatic degradation, and disulfides containing a penicillamine residue are approximately three times less susceptible to reduction by glutathione, a cellular reducing agent.

Despite this modification, the #6(L2)-Pen-Insulin conjugate also lowers blood glucose in STZ mice. While this may indicate that diminished in vitro activity does not result in diminished in vivo activity, there are a few interesting characteristics of this study. First, despite the dramatic drops in blood glucose, none of the animals in this study displayed the typical symptoms of hypoglycemia, seizures and hypothermia. Therefore, none of the animals needed to be rescued with glucose injections. In addition, while the 25 nmol/kg dose induced low blood glucose in these animals, no additional drops in blood glucose were observed when the dose was increased from 25 to 50 nmol/kg. It can also be seen that the #6(L2)-Pen-Insulin conjugate has a significantly extended duration of action, relative to native insulin. These observations may indicate that despite lowering blood glucose, the #6(L2)-Pen-Insulin conjugate appears to represent a “safer” insulin therapy.

1. A conjugate comprising
an insulin receptor antagonist peptide, wherein said antagonist peptide comprises a sequence of SLEEEWAQIQSEVWGRGSPSY (SEQ ID NO: 181), SX₂EEEWAQIQSEVWGRGSPSYC (SEQ ID NO: 182) or GSLDESFYDWFERQLG (SEQ ID NO: 183), or an analog of SEQ ID NO: 181 or 183 further modified to comprise a cysteine amino acid added at either the N-terminus or the C-terminus, wherein X₂ is a hydrophobic amino; and
an insulin agonist peptide, wherein the antagonist peptide is covalently linked to insulin agonist; said conjugate having similar potency, but reduced maximal activity, at the insulin receptor relative to native insulin.

2. (canceled)

3. The conjugate of claim 1 wherein the antagonist peptide comprises a sequence of SX₂EEEWAQIQSEVWGRGSPSYC (SEQ ID NO: 182) wherein X₂ is a hydrophobic amino and the antagonist peptide is linked to the insulin agonist peptide via a disulfide bond.

4. The conjugate of claim 3 wherein X₂ is selected from the group consisting of leucine, isoleucine, d-leucine and valine.

5. The conjugate of claim 4 wherein the insulin agonist peptide comprises an A chain and a B chain wherein said A chain comprises a sequence of GIVX₄X₅CCX₈X₉X₁₀CX₁₂LX₁₄X₁₅LX₁₇X₁₈YCX₂₁-R₅₃ (SEQ ID NO: 19), and said B chain comprises a sequence of R₆₂-X₂₅LCGX₂₉X₃₀LVX₃₃X₃₄LYLVCGX₄₁X₄₂GFX₄₅ (SEQ ID NO: 20), wherein
X₄ is glutamic acid or aspartic acid;
X₅ is glutamine or glutamic acid
X₈ is histidine, threonine or phenylalanine;
X₉ is serine, arginine, lysine, ornithine or alanine;
X₁₀ is isoleucine or serine;
X₁₂ is serine or aspartic acid;
X₁₄ is tyrosine, arginine, lysine, ornithine or alanine;
X₁₅ is glutamine, glutamic acid, arginine, alanine, lysine, ornithine or leucine;
X₁₇ is glutamic acid, aspartic acid, asparagine, lysine, ornithine or glutamine;
X₁₈ is methionine, asparagine, glutamine, aspartic acid, glutamic acid or threonine;
X₂₁ is selected from the group consisting of alanine, glycine, serine, valine, threonine, isoleucine, leucine, glutamine, glutamic acid, asparagine, aspartic acid, histidine, tryptophan, tyrosine, and methionine;
X₂₅ is histidine or threonine;
X₂₉ is selected from the group consisting of alanine, glycine and serine;
X₃₀ is selected from the group consisting of histidine, aspartic acid, glutamic acid, homocysteic acid and cysteic acid;
X₃₃ is selected from the group consisting of aspartic acid and glutamic acid;
X₃₄ is selected from the group consisting of alanine and threonine;
X₄₁ is selected from the group consisting of glutamic acid, aspartic acid or asparagine;
X₄₂ is selected from the group consisting of alanine, ornithine, lysine and arginine;
X₄₅ is tyrosine or phenylalanine;
R₆₂ is selected from the group consisting of AYRPSE (SEQ ID NO: 14), FVNQ (SEQ ID NO: 12), PGPE (SEQ ID NO: 11), a tripeptide glycine-proline-glutamic acid, a tripeptide valine-asparagine-glutamine, a dipeptide proline-glutamic acid, a dipeptide asparagine-glutamine, glutamine, glutamic acid and an N-terminal amine; and
R₅₃ is COOH or CONH₂.

6. The conjugate of claim 5 wherein said A chain comprises the sequence GIVEQCCX₈X₉ICSLYQLENYCX₂₁-R₅₃ (SEQ ID NO: 73) said B chain comprises the sequence R₆₂-X₂₅LCGX₂₉X₃₀LVX₃₃X₃₄LYLVCGX₄₁X₄₂GFX₄₅ (SEQ ID NO: 20), wherein
X₈ is histidine or threonine;
X₉ is serine, lysine, or alanine;
X₂₁ is alanine, glycine or asparagine;
X₂₅ is histidine or threonine;
X₂₉ is selected from the group consisting of alanine, glycine and serine;
X₃₀ is selected from the group consisting of histidine, aspartic acid, glutamic acid, homocysteic acid and cysteic acid;
X₃₃ is selected from the group consisting of aspartic acid and glutamic acid;
X₃₄ is selected from the group consisting of alanine and threonine;
X₄₁ is selected from the group consisting of glutamic acid, aspartic acid or asparagine;
X₄₂ is selected from the group consisting of alanine, ornithine, lysine and arginine;
X₄₅ is tyrosine or phenylalanine;
R₆₂ is selected from the group consisting of FVNQ (SEQ ID NO: 12), a tripeptide valine-asparagine-glutamine, a dipeptide asparagine-glutamine, glutamine and an N-terminal amine; and
R₅₃ is COOH or CONH₂.

7. The conjugate of claim 5 wherein said A chain comprises a sequence GIVDECCX₈X₉SCDLRRLEMX₁₉CX₂₁-R₅₃ (SEQ ID NO: 74) and said B chain comprises a sequence R₆₂-X₂₅LCGAX₃₀LVDALYLVCGDX₄₂GFY (SEQ ID NO: 75), wherein
X₈ is phenylalanine or histidine;
X₉ is arginine, ornithine or alanine;
X₁₉ is tyrosine, 4-methoxy-phenylalanine or 4-amino-phenylalanine;
X₂₁ is alanine or asparagine;
X₂₅ is histidine or threonine;
X₃₀ is selected from the group consisting of histidine, aspartic acid, glutamic acid, homocysteic acid and cysteic acid;
X₄₂ is selected from the group consisting of alanine ornithine and arginine;
and R₅₃ is COOH or CONH₂;
R₆₂ is selected from the group consisting of AYRPSE (SEQ ID NO: 14), FVNQ (SEQ ID NO: 12), PGPE (SEQ ID NO: 11), a tripeptide glycine-proline-glutamic acid, a tripeptide valine-asparagine-glutamine, a dipeptide proline-glutamic acid, a dipeptide asparagine-glutamine, glutamine, glutamic acid and an N-terminal amine; and
R₅₃ is COOH or CONH₂.

8. The conjugate of claim 5 wherein
the A chain comprises the sequence GIVEQCCTSICSLYQLENYCN (SEQ ID NO: 1) or GIVDECCRSCDLRRLEMYCA (SEQ ID NO: 5) and
the B chain sequence comprises the sequence FVKQX₂₅LCGSHLVEALYLVCGERGFF-R₆₃ (SEQ ID NO: 147), or FVNQX₂₅LCGSHLVEALYLVCGERGFF-R₆₃ (SEQ ID NO: 148), wherein
X₂₅ is selected from the group consisting of histidine and threonine; and
R₆₃ is selected from the group consisting of YTX₂₈KT (SEQ ID NO: 149), YTKPT (SEQ ID NO: 150), YTX₂₈K (SEQ ID NO: 152), YTKP (SEQ ID NO: 151), YTPK (SEQ ID NO: 70), YTX₂₈, YT, Y and a bond, wherein X₂₈ is proline, aspartic acid or glutamic acid.

9. The conjugate of claim 5 wherein
the A chain comprises the sequence GIVEQCCTSICSLYQLENYCN (SEQ ID NO: 1) or GIVDECCRSCDLRRLEMYCA (SEQ ID NO: 5); and
the B chain sequence comprises the sequence GPETLCGAELVDALYLVCGDRGFYFNKPT (SEQ ID NO: 6), FVKQX₂₅LCGSHLVEALYLVCGERGFFYTEKT (SEQ ID NO: 162), FVNQX₂₅LCGSHLVEALYLVCGERGFFYTDKT (SEQ ID NO: 164), FVNQX₂₅LCGSHLVEALYLVCGERGFFYTKPT (SEQ ID NO: 165) or FVNQX₂₅LCGSHLVEALYLVCGERGFFYTPKT (SEQ ID NO: 161) wherein X₂₅ is selected from the group consisting of histidine and threonine.

10. The conjugate of claim 9 wherein said A chain comprises a sequence GIVEQCCTSICSLYQLENYCN (SEQ ID NO: 1) and said B chain comprises a sequence FVNQHLCGSHLVEALYLVCGERGFFYTPKT (SEQ ID NO: 2).

11. The conjugate of claim 9 wherein the antagonist peptide is covalently bound to the carboxy terminus of the B chain.

12. The conjugate of claim 10 wherein the antagonist peptide is covalently bound via the side chain of a lysine residue present at position 28 or 29 of the insulin B chain.

13. The conjugate of claim 12 wherein the lysine residue present at position 28 or 29 of the insulin B chain is modified to comprise a side chain of Structure I: 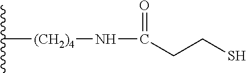
and the antagonist peptide is covalently bound via a disulfide linkage.

14. The conjugate of claim 13 further comprising a dipeptide element of the structure of Formula X: 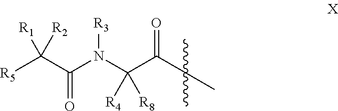
linked to said insulin peptide through an amide bond formed between said dipeptide element and an amine of the insulin A or B chain, wherein
R₁, R₂, R₄ and R₈ are independently selected from the group consisting of H, C₁-C₁₈ alkyl, C₂-C₁₈ alkenyl, (C₁-C₁₈ alkyl)OH, (C₁-C₁₈ alkyl)SH, (C₂-C₃ alkyl)SCH₃, (C₁-C₄ alkyl)CONH₂, (C1-C₄ alkyl)COOH, (C₁-C₄ alkyl)NH₂, (C1-C₄ alkyl)NHC(NH₂+)NH₂, (C₀-C₄ alkyl)(C₃-C₆ cycloalkyl), (C₀-C₄ alkyl)(C₂-C₅ heterocyclic), (C₀-C₄ alkyl)(C₆-C₁₀ aryl)R₇, (C₁-C₄ alkyl)(C₃-C₉ heteroaryl), and C₁-C₁₂ alkyl(W₁)C₁-C₁₂ alkyl, wherein W₁ is a heteroatom selected from the group consisting of N, S and O, or
R₁ and R₂ together with the atoms to which they are attached form a C₃-C₁₂ cycloalkyl or aryl; or
R₄ and R₈ together with the atoms to which they are attached form a C₃-C₆ cycloalkyl;
R₃ is selected from the group consisting of C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₅ heterocyclic), (C₀-C₄ alkyl)(C₆-C₁₀ aryl)R₇, and (C₁-C₄ alkyl)(C₃-C₉ heteroaryl) or R₄ and R₃ together with the atoms to which they are attached form a 4, 5 or 6 member heterocyclic ring;
R₅ is NHR₆ or OH;
R₆ is H, C₁-C₈ alkyl or R₆ and R₁ together with the atoms to which they are attached form a 4, 5 or 6 member heterocyclic ring; and
R₇ is selected from the group consisting of H, OH, C₁-C₁₈ alkyl, C₂-C₁₈ alkenyl, (C₀-C₄ alkyl)CONH₂, (C₀-C₄ alkyl)COOH, (C₀-C₄ alkyl)NH₂, (C₀-C₄ alkyl)OH, and halo.

15. The conjugate of claim 14 wherein
R₁ and R₂ are independently C₁-C₁₈ alkyl or aryl;
R₃ is C₁-C₁₈ alkyl or R₃ and R₄ together with the atoms to which they are attached form a pyrrolidine ring;
R₄ and R₈ are independently selected from the group consisting of hydrogen, and C₁-C₁₈ alkyl; and
R₅ is an amine or a hydroxyl.

16. The conjugate of claim 14, wherein
R₁ is hydrogen or C₁-C₈ alkyl;
R₃ is C₁-C₁₈ alkyl or R₃ and R₄ together with the atoms to which they are attached form a pyrrolidine ring;
R₂, R₄ and R₈ are each hydrogen; and
R₅ is NH₂.

17. The conjugate of claim 13, wherein an amino acid side chain of the conjugate is covalently attached to an acyl group or an alkyl group via an alkyl amine, amide, ether, ester, thioether, or thioester linkage, wherein said acyl group or alkyl group is non-native to a naturally occurring amino acid.

18. (canceled)

19. (canceled)

20. A method of reducing the risk of hypoglycemia associated with treating diabetes, said method comprising administering an effective amount of a conjugate of claim 13.

21. A pharmaceutical composition comprising the conjugate of claim 1, and a pharmaceutically acceptable carrier.

22. A method of treating diabetes, said method comprising administering an effective amount of a pharmaceutical composition of claim 21.

23. (canceled)

24. (canceled)

25. A conjugate comprising
an insulin receptor antagonist peptide; and an insulin agonist peptide, wherein
said antagonist peptide comprises a sequence of SX₂EEEWAQIQSEVWGRGSPSYC (SEQ ID NO: 182) wherein X₂ is selected from the group consisting of leucine, isoleucine, d-leucine and valine;
said insulin agonist peptide comprises an A chain sequence of GIVDECCX₈X₉SCDLRRLEMX₁₉CX₂₁-R₅₃ (SEQ ID NO: 74) and a B chain sequence selected from the group consisting of
(SEQ ID NO: 6)
GPETLCGAELVDALYLVCGDRGFYFNKPT,
(SEQ ID NO: 162)
FVKQX₂₅LCGSHLVEALYLVCGERGFFYTEKT,
(SEQ ID NO: 164)
FVNQX₂₅LCGSHLVEALYLVCGERGFFYTDKT,
(SEQ ID NO: 165)
FVNQX₂₅LCGSHLVEALYLVCGERGFFYTKPT
and
(SEQ ID NO: 161)
FVNQX₂₅LCGSHLVEALYLVCGERGFFYTPKT;
wherein
X₅ is phenylalanine or histidine;
X₉ is arginine, ornithine or alanine;
X₁₉ is tyrosine, 4-methoxy-phenylalanine or 4-amino-phenylalanine;
X₂₁ is alanine or asparagine; and
X₂₅ is selected from the group consisting of histidine and threonine;
wherein the A chain and B chain are bound together by disulfide bonds, and the lysine residue present at position 28 or 29 of the insulin B chain is modified to comprise a side chain of Structure I: 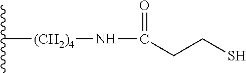
and the antagonist peptide is covalently linked via a disulfide linkage to the side chain of the lysine residue present at position 28 or 29 of the insulin B chain.
